SEQUENCE LISTING (90 sequences)

SEQ ID NO: 1 - protein, 541 aa, Pseudonocardia sp. N23
MTAAQAPDPAGVPLVERTVPRMLARSAALDPDRPFVVTRERTWSHTDAHRIVATLAAAFTDRGIGQGSRVAVMMPTSPRHVWLLLALAHLRAVPVALNPDASGEVLRYFVADSECVLGVVDQERAAAFATAAGPDGPPAIVLPPGADDLGELGSAGPGPLDPGAASFSDTFVVLYTSGSTGMPKATAVTHAQVITCGAVFTDRLGLGPADRLYTCLPLFHINATAYSLSGALVSGASLALGPHFSATTFWDDVADLGATEVNAMGSMVRILQSRPPRPAERAHRVRTMFVAPLPPDAVELSERFGLDFATCYAQTEWLPSSMTRPGEGYGRPGATGPVLPWTEVRIVGDDDRPLPAGQTGEIILRPRDPYTTFQGYLGKPQETVDAWRNLWFHTGDLGDIGPDGWLHYRGRRKDVIRRRGENIPATVVEDLLAGHPDIAEVAAVSVPAHISEEEIFAFVVPGAGAALTTADVEAHAHAVLPRYMVPSYLALVPDLPRTATNKIAKVELTERARAAVEGTGDPADAPTRTSAADRVVVPAAE

SEQ ID NO: 2 - protein, 548 aa, Pseudomonas putida
MMVPTLEHELAPNEANHVPLSPLSFLKRAAQVYPQRDAVIYGARRYSYRQLHERSRALASALERVGVQPGERVAILAPNIPEMLEAHYGVPGAGAVLVCINIRLEGRSIAFILRHCAAKVLICDREFGAVANQALAMLDAPPLLVGIDDDQAERADLAHDLDYEAFLAQGDPARPLSAPQNEWQSIAINYTSGTTGDPKGVVLHHRGAYLNACAGALIFQLGPRSVYLWTLPMFHCNGWSHTWAVTLSGGTHVCLRKVQPDAINAAIAEHAVTHLSAAPVVMSMLIHAEHASAPPVPVSVITGGAAPPSAVIAAMEARGFNITHAYGMTESYGPSTLCLWQPGVDELPLEARAQFMSRQGVAHPLLEEATVLDTDTGRPVPADGLTLGELVVRGNTVMKGYLHNPEATRAALANGWLHTGDLAVLHLDGYVEIKDRAKDIIISGGENISSLEIEEVLYQHPEVVEAAVVARPDSRWGETPHAFVTLRADALASGDDLVRWCRERLAHFKAPRHVSLVDLPKTATGKIQKFVLREWARQQEAQIADAEH

SEQ ID NO: 3 - protein, 549 aa, Streptomyces sp.ADI96-02
MLSTMQDVPLTVTRILQHGMTIHGKSQVTTWTGEPEPHRRTFAEIGARATRLAHALRDELGIDGDQRVATLMWNNAEHVEAYLAVPSMGAVLHTLNLRLPAEQLIWIVNHADDKVVIVNGSLLPLLVPLLPHLPTVEHVVVSGPGDRSALAGVAPRVHEYEELIADRPTTYDWPELDERQAAAMCYTSGTTGDPKGVVYSHRSVYLHSMQVNMTESMGLTDKDTTLVVVPQFHVNAWGLPHATFMAGVNMLMPDRFLQPAPLADMIERERPTHAAAVPTIWQGLLAEVTAHPRDLTSMASVTIGGAACPPSLMEAYDKLGVRLCHAWGMTETSPLGTMANPPAGLSAEEEWPYRVTQGRFPAGVEARLVGPAGDHLPWDGRSAGELEVRGAWIAGAYYGGADGEHLRPEDKFSADGWLKTGDVGVISADGFLTLTDRAKDVIKSGGEWISSVELENALMAHPDVAEAAVVAVPDEKWGERPLATVVLKEGAEVGYEALKVFLADSGIAKWQLPERWTVIPAVPKTSVGKFDKKVIRKQYADGELDITQL

SEQ ID NO: 4 - protein, 560 aa, Erythrobacter citreus LAMA 915
MSRAECRDRLTAPGERFEIETIDIRGVPTRVWKHAPTNMRQVAMAARTHGDRLFAIYEDERVTYEAWFRAVARMAAELRERGVAKGDRVALAMRNLPEWPVAFFAATTIGAICVPLNAWWTGPELAFGLANSGAKLLVCDAERWERIAPHRGELPDLEHALVSRSDAPLEGAEQLEDLLGTPKDYAALPSAALPQVDIDPEDEATIFYTSGTTGQPKGALGTHRNLCTNIMSSAYNGAIAFLRRGEEPPAPVQKVGLTVIPLFHVTACSAGLMGYVVAGHTMVFMHKWDPVKAFQLIEREKVNLTGGVPTIAWQLLEHPERANYDLSSLEAVAYGGAPAAPELVRKIHEEFGALPANGWGMTETMATVTGHSSEDYLNRPDSCGPPVAVADLKIVGDDGVTELPVGEVGELWARGPMVVKGYWNRPEATAETFVDGWVRTGDLARLDEEGWCYIVDRAKDMIIRGGENIYSSEVENVLYDHPAVTDAALVAIAHPTLGEEPAAVVHLAPGMSATEDELREWVAARLAKFKVPVRIAFVQDTLPRNANGKILKKDLGAFFA

SEQ ID NO: 5 - protein, 700 aa, Saccharomyces cerevisiae
MVAQYTVPVGKAANEHETAPRRNYQCREKPLVRPPNTKCSTVYEFVLECFQKNKNSNAMGWRDVKEIHEESKSVMKKVDGKETSVEKKWMYYELSHYHYNSFDQLTDIMHEIGRGLVKIGLKPNDDDKLHLYAATSHKWMKMFLGAQSQGIPVVTAYDTLGEKGLIHSLVQTGSKAIFTDNSLLPSLIKPVQAAQDVKYIIHFDSISSEDRRQSGKIYQSAHDAINRIKEVRPDIKTFSFDDILKLGKESCNEIDVHPPGKDDLCCIMYTSGSTGEPKGVVLKHSNVVAGVGGASLNVLKFVGNTDRVICFLPLAHIFELVFELLSFYWGACIGYATVKTLTSSSVRNCQGDLQEFKPTIMVGVAAVWETVRKGILNQIDNLPFLTKKIFWTAYNTKLNMQRLHIPGGGALGNLVFKKIRTATGGQLRYLLNGGSPISRDAQEFITNLICPMLIGYGLTETCASTTILDPANFELGVAGDLTGCVTVKLVDVEELGYFAKNNQGEVWITGANVTPEYYKNEEETSQALTSDGWFKTGDIGEWEANGHLKIIDRKKNLVKTMNGEYIALEKLESVYRSNEYVANICVYADQSKTKPVGIIVPNHAPLTKLAKKLGIMEQKDSSINIENYLEDAKLIKAVYSDLLKTGKDQGLVGIELLAGIVFFDGEWTPQNGFVTSAQKLKRKDILNAVKDKVDAVYSSS

SEQ ID NO: 6 - protein, 536 aa, Citreicella sp. SE45
MSLSTEETARRRTLAEGAGYDALREGFRWPGAARVNMAEQVCDSWAAREPGRPAILDMRAGGAPEVVSYGALQALSRRVEAWFRGQGVARGDRVGVLLSQSPLCAAAHIAAWRMGAISVPLFKLFKHDALESRLGDSGARVVVSDDEGAAMLAPFGLSVVTEAGLPQDGATEPAADTGPEDPAIIIYTSGTTGKPKGALHGHRVLTGHLPGVEMSHDLLGQPGDVLWTPADWAWIGGLFDVLMPGLYLGVPVVAARMPRFEISECLRICQQASVRNVFFPPTAFRMLKSEGAELPGLRSVASGGEPLGAEMLAWGRKAFGVEINEFYGQTECNMVASSCGALFRARPGCIGKPAPGFHIAVIDEDGNETDGEGDVAIRRGAGSMLLEYWQKPQETADKFRGDWLVTGDRGTWEDGYLRFVGREDDVITSAGYRIGPTEIEDCLMTHPAVATVGVVGKPCPLRTELVKAYVVLRPGVEVRASELQAWVKERLATYSYPREIAFLDALPMTVTGKVIRKELKAIAAAERTAEAAGEVS

SEQ ID NO: 7 - protein, 513 aa, Bacillus subtilis (strain 168)
MNLVSKLEETASEKPDSIACRFKDHMMTYQELNEYIQRFADGLQEAGMEKGDHLALLLGNSPDFIIAFFGALKAGIVVVPINPLYTPTEIGYMLTNGDVKAIVGVSQLLPLYESMHESLPKVELVILCQTGEAEPEAADPEVRMKMTTFAKILRPTSAAKQNQEPVPDDTAVILYTSGTTGKPKGAMLTHQNLYSNANDVAGYLGMDERDNVVCALPMFHVFCLTVCMNAPLMSGATVLIEPQFSPASVFKLVKQQQATIFAGVPTMYNYLFQHENGKKDDFSSIRLCISGGASMPVALLTAFEEKFGVTILEGYGLSEASPVTCFNPFDRGRKPGSIGTSILHVENKVVDPLGRELPAHQVGELIVKGPNVMKGYYKMPMETEHALKDGWLYTGDLARRDEDGYFYIVDRKKDMIIVGGYNVYPREVEEVLYSHPDVKEAVVIGVPDPQSGEAVKGYVVPKRSGVTEEDIMQHCEKHLAKYKRPAAITFLDDIPKNATGKMLRRALRDILPQ

SEQ ID NO: 8 - protein, 694 aa, Saccharomyces cerevisiae
MTEQYSVAVGEAANEHETAPRRNIRVKDQPLIRPINSSASTLYEFALECFTKGGKRDGMAWRDIIDIHETKKTIVKRVDGKDKPIEKTWLYYELTPYITMTYEEMICVMHDIGRGLIKIGVKPNGENKFHIFASTSHKWMKTFLGCMSQGIPVVTAYDTLGESGLIHSMVETDSVAIFTDNQLLSKLAVPLKTAKNVKFVIHNEPIDPSDKRQNGKLYKAAKDAVDKIKEVRPDIKIYSFDEIIEIGKKAKDEVELHFPKPEDPACIMYTSGSTGTPKGVVLTHYNIVAGIGGVGHNVIGWIGPTDRIIAFLPLAHIFELTFEFEAFYWNGILGYANVKTLTPTSTRNCQGDLMEFKPTVMVGVAAVWETVRKGILAKINELPGWSQTLFWTVYALKERNIPCSGLLSGLIFKRIREATGGNLRFILNGGSAISIDAQKFLSNLLCPMLIGYGLTEGVANACVLEPEHFDYGIAGDLVGTITAKLVDVEDLGYFAKNNQGELLFKGAPICSEYYKNPEETAAAFTDDGWFRTGDIAEWTPKGQVKIIDRKKNLVKTLNGEYIALEKLESIYRSNPYVQNICVYADENKVKPVGIVVPNLGHLSKLAIELGIMVPGEDVESYIHEKKLQDAVCKDMLSTAKSQGLNGIELLCGIVFFEEEWTPENGLVTSAQKLKRRDILAAVKPDVERVYKENT

SEQ ID NO: 9 - protein, 504 aa, Bhargavaea cecembensis DSE10
MYTDHGWIMKRADITPDGTALIDVHTGQRWTYRELAGRTAAYMEQFRSAGLRKGERVAVLSHNRIDLFAVLFACAGRGLIYVPMNWRLSESELRYIVSDSGPSLLLHDHEHAGRAAGLGIPAALLDSVPATSVNLRTEQAAGRLDDPWMMIYTGGTTGRPKGVVLTFESVNWNAINTIISWNLSARDCTLNYMPLFHTGGLNALSLPILMAGGTVVIGRKFDPEEAIRALNDYRTTISLFVPTMHQAMLDTDLFWESDFPTVDVFLSGGAPCPQTVYDAYRKKGVRFREGYGMTEAGPNNFIIDPDTAMRKRGAVGKSMQFNEVRILDAKGRPCRAGEVGELHLRGRHLFSHYWNNEEATQEALKEGWFSTGDLASRDEDGDYFIVGRKKEMIISGGENIYPQEVEQCLIGHDGVREIAVIGIADRKWGERVVAFIVAQPGNIPKTEELLKHCAQTLGSYKVPKDFFFVQELPITDIGKIDKKQLAIMAEELKKEEMQHPGQSG

SEQ ID NO: 10 - protein, 602 aa, Deltaproteobacteria bacterium ADurb.Bin022
MHKFTLDKPDNLVDWWGESVTRFADRPLFGTKNKEGVYKWATYKEIGNRIDNLRAGLTQLGIGKDDVVGIIANNRPEWAVIGFATWGCLARYVPMYEAELVQVWKYIINDSGAKVLFVSNPAIYEKIKDFPKDIPTLKHIFIIESDGDNSMASLEKKGAAKPVAPKSPKAEDVAELIYTSGTTGNPKGVLLMHMNFTSNSHAGLKMYPELYENEVVSLTILPWAHVFGQTAELFAIIRLGGRMGLIESTKTIINDIVQIKPTFIIAVPTVFNRIYDGLWNKMNKDGGLARALFVMGVEAAKKKRILAEKGQSDLMTNFKVAVADKIVFKKIRERMGGRMLGSMTGSAAMNVEISKFFFDIGIPIYDCYGLTETSPGITMNGSQAYRIGSVGRPIDKVKVVIDSSVVEEGATDGEIIAYGPNVMKGYHNRPEDTKAALTPDGGFRTGDRGRLDKDGYLFITGRIKEQYKLENGKFCFPVSLEENICLASFVQQAVVYGLNRPYNVCIVVPDFDVLLDYAKEKGLPTDIKTLVEREDIIHMISEAVTGQLKGKFGGYEIPKKFIILPEAFSLDNGMLTQTMKLKRKVILDKLNDRIEALYKEDK

SEQ ID NO: 11 - protein, 534 aa, Alcaligenes xylosoxydans
MYSRIHEPHACTLTDALREWAASRPAAPWLEDSQGIAFTVGQAFTSSQRFASFLHHQLGVQPEERVGVFMSNSCAMVATTFGIGYLRATAVMLNTELRSSFLRHQLNDCQLATIVVDSALVEHVASLADELPHLRTLVVVGDAPAAVPERWRQVAWMDSSACAPWEGPAPRPEDIFCIMYTSGTTGPSKGVLMPHCHCALLGLGAIRSLEITEADKYYICLPLFHANGLFMQLGATVLAGIPAFLKQRFSASTWLADIRRSGATLTNHLGTTAMFVINQPPTEQDRDHRLRASLSAPNPAQHEAVFRERFGVKDVLSGFGMTEVGIPIWGRIGHAAPNAAGWAHEDRFEICIADPETDVPVLAGQVGEILVRPKVPFGFMAGYLNVPAKTVEAWRNLWFHTGDAGTRDEQGLITFVDRIKDCIRRRGENISATEVEVVVGQLPGVHEVAAYAVPAQGAGGEDEVMLALVPSEGAALDMADIVRQASAQLPRFAKPRYLRQMDSLPKTATGKIQRAVLRQQGSAGAYDAEAAPAR

SEQ ID NO: 12 - protein, 518 aa, Novosphingobium sp. MD-1
MQFTQGLERAVQHHPDVTATICRARSQTFAELYERVTGLAGCLASRSLAKGARIAVLALNSDHYLEVYLATAWAGGVIVPVNFRWSPAEIAYSLNDAGCVALMVDQHHAALVPTLREQCPGLQHIFLMGGTEESDDLPGLDALIAAAEPLQNAGAGGDDLLGIFYTGGTTGRPKGVMLSHANLCSSGLSMLAEGVFNEGAVGLHVAPMFHLADMLLTTCLVLRGCTHVMLPAFSPDAVLDHVARFGVTDTLVVPAMLQAIVDHPAIGNFDTSSLCNILYGASPASETLLRRTMAAFPDVRLTQGYGMTESAAFICALPWHQHVVDNDGPNRLRAAGRSTFDVHLQIVDPDDRELPRGEIGEIIVKGPNVMQGYYNMPEATAETLRGGWLHTGDMAWMDEEGYVFIVDRAKDMIISGGENIYSAEVENAVASHPAVAANAVIGIPHEQMGEAVHVALVLRPGSELSLEALQAHCRALIAGYKVPRSMEVRPSLPLSGAGKILKTELREPFWKGRDRAVG

SEQ ID NO: 13 - protein, 720 aa, Arabidopsis thaliana
MEDSGVNPMDSPSKGSDFGVYGIIGGGIVALLVPVLLSVVLNGTKKGKKRGVPIKVGGEEGYTMRHARAPELVDVPWEGAATMPALFEQSCKKYSKDRLLGTREFIDKEFITASDGRKFEKLHLGEYKWQSYGEVFERVCNFASGLVNVGHNVDDRVAIFSDTRAEWFIAFQGCFRQSITVVTIYASLGEEALIYSLNETRVSTLICDSKQLKKLSAIQSSLKTVKNIIYIEEDGVDVASSDVNSMGDITVSSISEVEKLGQKNAVQPILPSKNGVAVIMFTSGSTGLPKGVMITHGNLVATAAGVMKVVPKLDKNDTYIAYLPLAHVFELEAEIVVFTSGSAIGYGSAMTLTDTSNKVKKGTKGDVSALKPTIMTAVPAILDRVREGVLKKVEEKGGMAKTLFDFAYKRRLAAVDGSWFGAWGLEKMLWDALVFKKIRAVLGGHIRFMLVGGAPLSPDSQRFINICMGSPIGQGYGLTETCAGATFSEWDDPAVGRVGPPLPCGYVKLVSWEEGGYRISDKPMPRGEIVVGGNSVTAGYFNNQEKTDEVYKVDEKGTRWFYTGDIGRFHPDGCLEVIDRKKDIVKLQHGEYVSLGKVEAALGSSNYVDNIMVHADPINSYCVALVVPSRGALEKWAEEAGVKHSEFAELCEKGEAVKEVQQSLTKAGKAAKLEKFELPAKIKLLSEPWTPESGLVTAALKIKREQIKSKFKDELSKLYA

SEQ ID NO: 14 - protein, 534 aa, Bradyrhizobium sp. CI-41S
MDWSQHAIPPMRLEPRFGDRVVPAFVDRPASLWAMIADAVAQNGGGEALVCGDIRISWHEVARRAAKVAAGFAKLGLNSGDRVAILLGNRIEFVLTMFAAAHAGLVTVLLSTRQQKPEIAYVLNDCGARALVHEATLAERIPDAADIPGLAHRIAVSDDAASQFAVLLDHPPAPAPAAVSEEDTAMILYTSGTTGRPKGAMLAHCNIIHSSMVFASTLRLTQADRSIAAVPLAHVTGAVANITTMVRCAGTLIIMPEFKAAEYLKVAARERVSYTVMVPAMYNLCLLQPDFDSYDLSSWRIGGFGGAPMPVATIERLDAKIPGLKLANCYGATETTSPSTLMPGELTAAHIDSVGLPCPGAEIIVMGPDGRELPRGEIGELWIRSASVIKGYWNNPKATAESFTDGFWHSGDLGSVDAENFVRVFDRQKDMINRGGLKIYSAEVESVLAGHPAVIESAIIAKPCPVLGERVHAVIVTRTEVDAESLRAWCAERLSDYKVPETMTLTTTPLPRNANGKVVKRQLRETLAAGQAPA

SEQ ID NO: 15 - protein, 612 aa, Bradyrhizobium sp. CI-41S
MAGPAVLTVADTIARSFLLAVQTRGDRPAIREKKFGIWQPTSWREWLQISKDIAHGLHASGFRPGDVASIIANAVPEWVYADMGILCAGGVSSGIYPTDSTAQVEYLVNDSRTKIVFVEDEEQLDKVLACRARCPTLEKIVVFDMEGLSGFSDPMVLSFAEFAALGRNHAHGNAALWDEMTGSRTASDLAILVYTSGTTGPPKGAMHSNRSVTHQMRHANDLFPSTDSEERLVFLPLCHVAERVGGYYISIALGSVMNFAESPETVPDNLREVQPTAFLAVPRVWEKFYSGITIALKDATPFQNWMYGRALAIGNRMTECRLEGETPPLSLRLANRAAYWLVFRNIRRMLGLDRCRIALTGAAPISPDLIRWYLALGLDMREVYGQTENCGVATIMPTERIKLGSVGKAAPWGEVMICPKGEILIKGDFLFMGYLNQPERTAETIDAKGWLHTGDVGTIDNEGYVRITDRMKDIIITSGGKNVTPSEIENQLKFSPYVSDAVVIGDKRPYLTCLIMIDQENVEKFAQDHDIPFTNYASLCRAREIQDLIQREVEAVNTKFARVETIKKFYLIERQLTPEDEELTPTMKLKRSFVNKRYAAEIDAMYGARAVA

SEQ ID NO: 16 - protein, 541 aa, Thermus thermophilus
MEGERMNAFPSTMMDEELNLWDFLERAAALFGRKEVVSRLHTGEVHRTTYAEVYQRARRLMGGLRALGVGVGDRVATLGFNHFRHLEAYFAVPGMGAVLHTANPRLSPKEIAYILNHAEDKVLLFDPNLLPLVEAIRGELKTVQHFVVMDEKAPEGYLAYEEALGEEADPVRVPERAACGMAYTTGTTGLPKGVVYSHRALVLHSLAASLVDGTALSEKDVVLPVVPMFHVNAWCLPYAATLVGAKQVLPGPRLDPASLVELFDGEGVTFTAGVPTVWLALADYLESTGHRLKTLRRLVVGGSAAPRSLIARFERMGVEVRQGYGLTETSPVVVQNFVKSHLESLSEEEKLTLKAKTGLPIPLVRLRVADEEGRPVPKDGKALGEVQLKGPWITGGYYGNEEATRSALTPDGFFRTGDIAVWDEEGYVEIKDRLKDLIKSGGEWISSVDLENALMGHPKVKEAAVVAIPHPKWQERPLAVVVPRGEKPTPEELNEHLLKAGFAKWQLPDAYVFAEEIPRTSAGKFLKRALREQYKNYYGGA

SEQ ID NO: 17 - protein, 524 aa, Microbacterium oxydans
MVRSTYPDVEIPEVSIHDFLFGDLSEAELDTVALVDGMSGATTTYRQLVGQIDLFAGALAARGVGVGTTVGVLCPNVPAFATVFHGILRAGATATTINSLYTADEIANQLTDAGATWLVTVSPLLPGAQAAAEKLGFDADHVIVLDGAEGHPSLPALLGEGRQAPDVSFDPSTHLAVLPYSSGTTGRPKGVMLTHRNLVANVSQCQPVLGVDASDRVLAVLPFFHIYGMTVLLNFALRQRAGLATMPRFDLPEFLRIIAEHRTSWVFVAPPIAVALAKHPIVDQYDLSAVKVIFSGAAPLDGTLASAVANRLGCIVTQGYGMTETSPAVNLISEARTEIDRSTIGPLVPNTEARLVDPDSGEDVVVPAEGASEPGELWVRGPQVMVGYLNRPDATAEMLDADGWLHTGDVATVTHDGIYRIVDRLKELIKYKGYQVAPAVLEAVLLEHPAIADAAVIGAFDDDGQEVPKAFVVRQPDADLDADAVMAHVTSHVAPHEKVRQVEFIDVIPKSSSGKILRKDLRAR

SEQ ID NO: 18 - protein, 665 aa, Arabidopsis thaliana
MSLAADNVLLVEEGRPATAEHPSAGPVYRCKYAKDGLLDLPTDIDSPWQFFSEAVKKYPNEQMLGQRVTTDSKVGPYTWITYKEAHDAAIRIGSAIRSRGVDPGHCCGIYGANCPEWIIAMEACMSQGITYVPLYDSLGVNAVEFIINHAEVSLVFVQEKTVSSILSCQKGCSSNLKTIVSFGEVSSTQKEEAKNQCVSLFSWNEFSLMGNLDEANLPRKRKTDICTIMYTSGTTGEPKGVILNNAAISVQVLSIDKMLEVTDRSCDTSDVFFSYLPLAHCYDQVMEIYFLSRGSSVGYWRGDIRYLMDDVQALKPTVFCGVPRVYDKLYAGIMQKISASGLIRKKLFDFAYNYKLGNMRKGFSQEEASPRLDRLMFDKIKEALGGRAHMLLSGAAPLPRHVEEFLRIIPASNLSQGYGLTESCGGSFTTLAGVFSMVGTVGVPMPTVEARLVSVPEMGYDAFSADVPRGEICLRGNSMFSGYHKRQDLTDQVLIDGWFHTGDIGEWQEDGSMKIIDRKKNIFKLSQGEYVAVENLENTYSRCPLIAQIWVYGNSFESFLVGVVVPDRKAIEDWAKLNYQSPNDFESLCQNLKAQKYFLDELNSTAKQYQLKGFEMLKAIHLEPNPFDIERDLITPTFKLKRPQLLQHYKGIVDQLYSEAKRSMA

SEQ ID NO: 19 - protein, 559 aa, Brevibacterium yomogidense
MSWFDERPWLRTLGLTETEAVPLEPSTPLRDLADTVAAHPTTAAWTHYGQSATYAEFDRQTTAFAAYLAESGIRPGDAVAVYAQNSPHFPIATYGIWKAGAVVVPLNPMYRDELTHAFADADVKAIVVQKALYLMRVKEYAADLPLVVLAGDLDWAQDGPDAVFGAYADLPDVPLPDLRTVVDERLDTDFEPLTVRPEDPALIGYTSGTSGKAKGALHPHSSISSNSRMAARNAGLPQGAGVVSLAPLFHITGFICQMIASTANGSTLVLNHRFDPASFLDLLRQEKPAFMAGPATVYTAMMASPSFGADAFDSFHSIMSGGAPLPEGLVKRFEEKTGHYIGQGYGLTETAAQAVTVPHSLRAPVDPESGNLSTGLPQRDAMVRILDDDGNPVGPREVGEVAISGPMVATEYLGNPQATADSLPGGELRTGDVGFMDPDGWVFIVDRKKDMINASGFKVWPREVEDILYMHPAVREGAVVGVPDEYRGETVVAFVSLQPDSQATAEDIIAHCKEHLASYKAPVEVTIVDELPKTSSGKILRRTVRDEATQARQAQPDAH

SEQ ID NO: 20 - protein, 555 aa, Nocardioides simplex
MSFRYYRDLHPTFADRTEWALPTVLRHHAAERPDAVWLDCPEEGRTWTFAETLTAAERVGRSLLAAGAEPGDRVVLVAQNSSAFVRTWLGTAVAGLVEVPVNTAYEHDFLAHQVSTVEATLAVVDDVYAARFVAIAEAAKSIRKFWVIDTGSRDQALATLRDAGWEAAPFEELDEAATAPEVVDATLALPDVRPQDLASVLFTSGTTGPSKGVAMPHAQMYFFADECVSLVRLTPDDAWMSVTPLFHGNAQFMAAYPTLVAGARFVTRSRFSASRWVDQLRESRVTVTNFIGVMMDFIWKQDRRDDDADNPLRVVFAAPTAATLVGPMSERYGIEAFVEVFGLTETSAPIISPYGVDRPAGAAGLAADEWFDVRLVDPETDEEVGVGEIGELVVRPKVPFICSMGYFNMPDKTVEAWRNLWFHTGDALRRDEDGWFYFVDRFKDALRRRGENISSYEIETSILAHPAVVECAVIAVPASSEAGEDEVMAYVITGGDAPVPTPAELWAHCDGRIPSFAVPRYLRFVDEMPKTPSQRVQKAKLRALGVTPDTHDREA

SEQ ID NO: 21 - protein, 568 aa, Brevibacterium linens
MTVTEEFRAARDKLIELRSDYDAAREQFEWPRFDHFNFALDWFDKIAENNDKPALWIVEQDGSEGKWSFAELSARSNQVANHFRRAGIKRGDHVMVMLNNQVELWETMLAGIKLGAVLMPATTQLGPIDLTDRAERGHAEFVVAGAEDAAKFDDVDVEVVRIVVGGEPTRQQDYSYSDADDESTEFDPQGSSRADDLMLLYFTSGTTSKAKMVAHTHVSYPVGHLSTMYWMGLTPGDVHLNVASPGWAKHAWSNIFTPWIAEACVFLYNYSRFDANALMETMDRVGVTSFCAPPTVWRMLIQADLKHLKTPPTKALGAGEPLNPEIIDRVHSDWGVLIRDGFGQTESTLQIGNSPDQELKYGSMGKALPGFDVVLIDPATGEEGDEGEICLRLDPRPIGLTTGYWSNPEKTAEAFEGGVYHTGDVASRDEDGFITYVGRADDVFKASDYRLSPFELESVLIEHEAVAEAAVVPSPDPVRLAVPKAYVVVSSKFDADAETARSILAYCREHLAPYKRIRRLEFAELPKTISGKIRRVELRAREDQLHPFSGEPVVEGNEYADTDFDLKS

SEQ ID NO: 22 - protein, 515 aa, Pseudomonas putida
MNLGKIITRSARYWPDHTAVADSQTRLTYAQLERRSNRLASGLGALGVATGEHVAILAANRVELVEAEVALYKAAMVKVPINARLSLDEVVRVLEDSCSVALITDATFAQALAERRAALPMLRQVIALEGEGGDLGYAALLERGSEAPCSLDPADDALAVLHYTSGSSGVLKAAMLSFGNRKALVRKSIASPTRRSGPDDVMAHVGPITHASGMQIMPLLAVGACNLLLDRYDDRLLLEAIERERVTRLFLVPAMINRLVNYPDVERFDLSSLKLVMYGAAPMAPALVKKAIELFGPILVQGYGAGETCSLVTVLTEQDHLIEDGNYQRLASCGRCYFETDLRVVNEAFEDVAPGEIGEIVVKGPDIMQGYWRAPALTAEVMRDGYYLTGDLATVDAQGYVFIVDRKKEMIISGGFNVYPSEVEQVIYGFPEVFEAAVVGVPDEQWGEAVRAVVVLKPGAQLDAAELIERCGRALAGFKKPRGVDFVTELPKNPNGKVVRRLVREAYWQHSDRRI

SEQ ID NO: 23 - protein, 681 aa, Drosophila melanogaster
MNDLKPATSYRSTSLHDAVKLRLDEPSSFSQTVPPQTIPEFFKESCEKYSDLPALVWETPGSGNDGWTTLTFGEYQERVEQAALMLLSVGVEERSSVGILAFNCPEWFFAEFGALRAGAVVAGVYPSNSAEAVHHVLATGESSVCVVDDAQQMAKLRAIKERLPRLKAVIQLHGPFEAFVDHEPGYFSWQKLQEQTFSSELKEELLARESRIRANECAMLIFTSGTVGMPKAVMLSHDNLVFDTKSAAAHMQDIQVGKESFVSYLPLSHVAAQIFDVFLGLSHAGCVTFADKDALKGTLIKTFRKARPTKMFGVPRVFEKLQERLVAAEAKARPYSRLLLARARAAVAEHQTTLMAGKSPSIYGNAKYWLACRVVKPIREMIGVDNCRVFFTGGAPTSEELKQFFLGLDIALGECYGMSETSGAITLNVDISNLYSAGQACEGVTLKIHEPDCNGQGEILMRGRLVFMGYLGLPDKTEETVKEDGWLHSGDLGYIDPKGNLIISGRLKELIITAGGENIPPVHIEELIKKELPCVSNVLLIGDHRKYLTVLLSLKTKCDAKTGIPLDALREETIEWLRDLDIHETRLSELLNIPADLQLPNDTAALAATLEITAKPKLLEAIEEGIKRANKYAISNAQKVQKFALIAHEFSVATGELGPTLKIRRNIVHAKYAKVIERLYK

SEQ ID NO: 24 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 25 - protein, 390 aa, Dendrobium catenatum
MPSLESIRKAPRANGFASILAIGRANPENFIEQSTYPDFFFRITNSEHLVDLKKKFQRICDKTAIRKRHFVWNEEFITTNPCLHTFMDKSLDVRQEVAIREIPKLGAKAAAKAIQEWGQPKSRITHLIFCTTSGMDLPGADYQLTQILGLNPNVERVMLYQQGCFAGGTTLRLAKCLAESRKGARVLVVCAETTTVLFRGPSEEHQEDLVTQALFADGASALIVGADPDEAAHERASFVIVSTSQVLLPDSAGAIGGHVSEGGLLATLHRDVPKIVSKNVEKCLEEAFTPFGITDWNSIFWVPHPGGRAILDLVEERVGLKPEKLLVSRHVLAEYGNMSSVCVHFALDEMRKRSAIEGKATTGEGLEWGVVFGFGPGLTVETVVLRSVPL

SEQ ID NO: 26 - protein, 497 aa, Dictyostelium
MNNSNVKSSPSIVKEEIVTLDKDQQPLLLKEHQHIIISPDIRINKPKRESLIRTPILNKFNQITESIITPSTPSLSQSDVLKTPPIKSLNNTKNSSLINTPPIQSVQQHQKQQQKVQVIQQQQQPLSRLSYKSNNNSFVLGIGISVPGEPISQQSLKDSISNDFSDKAETNEKVKRIFEQSQIKTRHLVRDYTKPENSIKFRHLETITDVNNQFKKVVPDLAQQACLRALKDWGGDKGDITHIVSVTSTGIIIPDVNFKLIDLLGLNKDVERVSLNLMGCLAGLSSLRTAASLAKASPRNRILVVCTEVCSLHFSNTDGGDQMVASSIFADGSAAYIIGCNPRIEETPLYEVMCSINRSFPNTENAMVWDLEKEGWNLGLDASIPIVIGSGIEAFVDTLLDKAKLQTSTAISAKDCEFLIHTGGKSILMNIENSLGIDPKQTKNTWDVYHAYGNMSSASVIFVMDHARKSKSLPTYSISLAFGPGLAFEGCFLKNVV

SEQ ID NO: 27 - protein, 497 aa, Arachis hypogaea; identical to SEQ ID NO: 26

SEQ ID NO: 28 - protein, 394 aa, Spinacia oleracea
MASVDISEIHNVERAKGQANVLAIGTANPPNVMYQADYPDFYFRLTNSEHMTDLKAKFKRICEKTTIKKRYMHISEDILKEKPDLCDYNASSLDIRQVILAKEVPKVGKDAAMKAIEEWGQAMSKITHLIFCTTSGVDIPGADYQLTMLLGLNPSVKRYMLCQQGCHAGGTVLRLAKDLAENNYGSRVLVVCSENTTVCFRGPTETHPDSMVAQALFADGAGAVIVGAYPDESLNERPIFQIVSTAQTILPNSQGAIEGHLRQIGLAIQLLPNVPDLISNNIDKCLVEAFNPIGINDWNSIFWIAHPGGPAILGQVESKLGLQESKLTTTWHVLREFGNMSSACVFFIMDETRKRSLKEGKTTTGDGFDWGVLFGFGPGLTVETVVLRSFPLNQ

SEQ ID NO: 29 - protein, 389 aa, Elaeis guineensis
MSGLSRDMNPSLERSVGRAAVLGIGTANPPHVVEQSTFPDYYFKITNSEHMAHLKEKFTRICEKSKIAKRYTVLTDEFLVANPTLTSFNAPSLDTRQQLLDVEVPRLGAEAATRAIKDWGRPMSDLTHLIFCNSYGASIPGADYELVKLLGLPLSTRRVMLYQQCCYAGGTVIRLAKDLAENNRDARVLVVCCELNTVGIRGPCQSHLEDLVSQALFGDGAGALIIGADPRAGVERSIFEIVRTSQNIIAGSEGALVAKLREVGLVGRLKPEIPMHLSCSIEKLASEALNPVGIADWNEAFWVMHPGGRAILDELEKKLGLGEEKLAATREVLRDYGNMSSTSVLFVMEVMRRRSEERGLATAGEGLEWGVLLGFGPGLTMETVVLRCP

SEQ ID NO: 30 - protein, 392 aa, Vitis pseudoreticulata
MALVEEIRNAQRAKGPATVLAIGTATPDNCLYQSDFADYYFRVTKSEHMTELKKKFNRICDKSMIKKRYIHLTEEMLEEHPNIGAYMAPSLNIRQEIITAEVPKLGKEAALKALKEWGQPKSKITHLVFCTTSGVEMPGADYKLANLLGLEPSVRRVMLYHQGCYAGGTVLRTAKDLAENNAGARVLVVCSEITVVTFRGPSENALDSLVGQALFGDGSAAVIVGSDPDISIERPLFQLVSAAQTFIPNSAGAIAGNLREVGLTFQLWPNVPTLISENIEKCLTKAFDPIGISDWNSLFWIAHPGGPAILDAVEAKLNLDKQKLKATRHILSEYGNMSSACVLFILDEMRKKSLKEGKTTTGEGLDWGVLFGFGPGLTIETVVLHSVQMDSN

SEQ ID NO: 31 - protein, 392 aa, Cannabis sativa
MASISVDQIRKAQRANGPATVLAIGTANPPTSFYQADYPDFYFRVTKNQHMTELKDKFKRICEKTTIKKRHLYLTEDRLNQHPNLLEYMAPSLNTRQDMLVVEIPKLGKEAAMKAIKEWGQPKSRITHLIFCSTNGVDMPGADYECAKLLGLSSSVKRVMLYQQGCHAGGSVLRIAKDLAENNKGARILTINSEITIGIFHSPDETYFDGMVGQALFGDGASATIVGADPDKEIGERPVFEMVSAAQEFIPNSDGAVDGHLTEAGLVYHIHKDVPGLISKNIEKSLVEALNPIGISDWNSLFWIVHPGGPAILNAVEAKLHLKKEKMADTRHVLSEYGNMSSVSIFFIMDKLRKRSLEEGKSTTGDGFEWGVLFGFGPGLTVETIVLHSLAN

SEQ ID NO: 32 - protein, 391 aa, Chenopodium quinoa
MASVQEIRNAQRADGPATILAIGTANPPNEMYQAEYPDFYFRVTESEHMTDLKKKFKRMCERSMIKKRYMHVTEELLKENPHMCDYNASSLNTRQDILATEVPKLGKEAAIKAIKEWGQPRSKITHVIFCTTSGVDMPGADYQLTKLLGLRPSVKRFMLYQQGCYAGGTVLRLAKDIAENNRGARVLVVCAEITVICFRGPTETHLDSMIGQALFGDGAGAVIVGADVDESIERPIFQLVWAAQTILPDSEGAIDGHLREVGLAFHLLKDVPGLISKNIEKALVEAFKPIGIDDWNSIFWVAHPGGPAILDQVESKLELKQDKLRDTRHVLSEFGNMSSACVLFILDEMRNRSLKEGKTTTGEGLDWGVLFGFGPGLTVETVMLHSVPITN

SEQ ID NO: 33 - protein, 389 aa, Ziziphus jujuba
MVTVDEIREAQRAKGPATIMAIGTATPPNAIDQSTFTDYYFRITNSDHKTDLKKKFKTICDKSMIKKRYLYLTEEHLKQNPNMSEYMAPSLDVRQEIVIAEVPKLGKEAANKAIKEWGQPKSKITHLVFSTISGVDAPGADYQLTKLLGLNPSVKRIMVYQQGCFAGGTSLRLAKDLAENNKGARVLVVCTEISAINFRGPSETYFDSNVGQILFGDGASAVVVGSDPLVGVEKPLFELVSASQTIIPDSEGNIEGHICEVGLTIRLSKKVPSLISNNIEKSLVEAFNPLGISDWNSIFWIAHPGGPAILDQIELKLGLKPEKLRASRHVLSEYGNMSSATVLFILDEMRKKSIEDGLKTPGEGLEWGVLFGFGPGLTVETVVLHSVTA

SEQ ID NO: 34 - protein, 319 aa, Marchantia polymorpha
MSRSRLIAQAVGPATVLAMGKAVPANVFEQATYPDFFFNITNSNDKPALKAKFQRICDKSGIKKRHFYLDQKILESNPAMCTYMETSLNCRQEIAVAQVPKLAKEASMNAIKEWGRPKSEITHIVMATTSGVNMPGAELATAKLLGLRPNVRRVMMYQQGCFAGATVLRVAKDLAENNAGARVLAICSEVTAVTFRAPSETHIDGLVGSALFGDGAAAVIVGSDPRPGIERPIYEMHWAGEMVLPESDGAIDGHLTEAGLVFHLLKDVPGLITKNIGGFLKDTKNLVGASSWNELFWAVHPGGPAILDQVEAKLELEKG

SEQ ID NO: 35 - protein, 393 aa, Caragana korshinskii
MAYLEEIREVQRARGPATILAIGTANPSNCIYQADFTDYYFRVTNSDHMTKLKAKLKRICENSMIKKRHVHLTEEILKENPNICTYKESSLDARQDMLIVEVPKLGEKAASKAIEEWGRPKSEITHLIFCSTSGVDMPGADYQLINLLGLKPSTKRFMLYHQGCFAGGTVLRLAKDLAENNAGARVLVVCSEITVVTFRGPSETHLDCLVGQALFGDGASSVIVGSDPDTSIERPLFHLVSASETILPNSEGAIEGHLREAGLMFQLKENVPQLIGENIEKSLEEMFHPLGISDWNSLFWISHPGGPAILKRIEETAGLNPEKLKATKHVLSEYGNMSSACVLFILDEMRKRSMEEGKSTTGEGLNWGVLFGFGPGLTMETIALHSANIDTGY

SEQ ID NO: 36 - protein, 389 aa, Glycine max
MVSVAEIRQAQRAEGPATILAIGTANPPNCVAQSTYPDYYFRITNSEHMTELKEKFQRMCDKSMIKRRYMYLNEEILKENPNMCAYMAPSLDARQDMVVVEVPKLGKEAAVKAIKEWGQPKSKITHLIFCTTSGVDMPGADYQLTKQLGLRPYVKRYMMYQQGCFAGGTVLRLAKDLAENNKGARVLVVCSEITAVTFRGPSDTHLDSLVGQALFGDGAAAVIVGSDPIPQVEKPLYELVWTAQTIAPDSEGAIDGHLREVGLTFHLLKDVPGIVSKNIDKALFEAFNPLNISDYNSIFWIAHPGGPAILDQVEQKLGLKPEKMKATRDVLSEYGNMSSACVLFILDEMRRKSAENGLKTTGEGLEWGVLFGFGPGLTIETVVLRSVAI

SEQ ID NO: 37 - protein, 394 aa, Humulus lupulus
MASVTVEQIRKAQRAEGPATILAIGTAVPANCFNQADFPDYYFRVTKSEHMTDLKKKFQRMCEKSTIKKRYLHLTEEHLKQNPHLCEYNAPSLNTRQDMLVVEVPKLGKEAAINAIKEWGQPKSKITHLIFCTGSSIDMPGADYQCAKLLGLRPSVKRVMLYQLGCYAGGKVLRIAKDIAENNKGARVLIVCSEITACIFRGPSEKHLDCLVGQSLFGDGASSVIVGADPDESVGERPIFELVSAAQTILPNSDGAIAGHVTEAGLTFHLLRDVPGLISQNIEKSLIEAFTPIGINDWNNIFWIAHPGGPAILDEIEAKLELKKEKMKASREMLSEYGNMSCASVFFIVDEMRKQSSKEGKSTTGDGLEWGALFGFGPGLTVETLVLHSVPTNV

SEQ ID NO: 38 - protein, 389 aa, Humulus lupulus
MVTVEEVRKAQRAEGPATILAIGTATPANCILQSEYPDYYFRITNSEHKTELKEKFKRMCDKSMIRKRYMHLTEEILKENPNLCAYEAPSLDARQDMVVVEVPKLGKEAATKAIKEWGQPKSKITHVVFCTTSGVDMPGADYQLTKLLGLRPSVKRLMMYQQGCFAGGTVLRVAKDLAENNKGARVLVVCSEITAVTFRGPNDTHLDSLVGQALFGDGSAALIIGADPTPEIEKPIFELVSAAQTILPDSDGAIDGHLREVGLTFHLLKDVPGLISKNIEKSLVEAFKPLGISDWNSLFWIAHPGGPAILDQVESKLALKPEKLRATRHVLGEYGNMSSACVLFILDEMRRKCAEDGLKTTGEGLEWGVLFGFGPGLTVETVVLHSVGI

SEQ ID NO: 39 - protein, 396 aa, Trema orientale
MASVTVDEIRKAQRAEGPATVLAIGTATPHNCVSQADYPDYYFRITNSEHMTELKEKFKRMCEKSMIKKRYMHLTEEILKENPKMCEYMAPSLDARQDMVVVEVPKLGKEAATKAIKEWGLPKSKITHLVFCTTSGVDMPGADYQLTKLLGLRPSVKRLMMYQQGCFAGGTVLRLAKDLAENNRGARVLVVCSEITAVTFRGPSDTHLDSMVGQALFGDGAAAVIVGADPDPSAGERPLFEMVSAAQTILPDSEGAIDGHLREAGLTFHLLKDVPGLISKNIEKSLTEAFSPLGISDWNSLFWIAHPGGPAILDQVEAKLKLKEEKLRATRHVLSEYGNMSSACVLFILDEMRKKSAEDGKPTTGEGLDWGVLFGFGPGLTVETVVLHSVAATATN

SEQ ID NO: 40 - protein, 396 aa, Plumbago indica
MAPAVQSQSHGGAYRSNGERSKGPATVLAIATAVPPNVYYQDEYADFFFRVTNSEHKTAIKEKFNRVCGTSMIKKRHMYFTEKMLNQNKNMCTWDDKSLNARQDMVIPAVPELGKEAALKAIEEWGKPLSNITHLIFCTTAGNDAPGADFRLTQLLGLNPSVNRYMIYQQGCFAGATALRIAKDLAENNKGARVLIVCCEIFAFAFRGPHEDHMDSLICQLLFGDGAAAVIVGGDPDETENALFELEWANSTIIPQSEEAITLRMREEGLMIGLSKEIPRLLGEQIEDILVEAFTPLGITDWSSLFWIAHPGGKAILEALEKKIGVEGKLWASWHVLKEYGNLTSACVLFAMDEMRKRSIKEGKATTGDGHEYGVLFGVGPGLTVETVVLKSVPLN

SEQ ID NO: 41 - protein, 398 aa, Artemisia annua
MASLTDIAAIREAQRAQGPATILAIGTANPANCVYQADYPDYYFRITKSEHMVDIKEKFKRMCDKSMIRKRYMHLTEEYLKENPSLCEYMAPSLDARQDVVVVEVPKLGKEAATKAIKEWGQPKSKITHLIFCTTSGVDMPGADYQLTKLLGLRPSVKRFMMYQQGCFAGGTVLRLAKDLAENNKDARVLVVCSEITAVTFRGPNDTHLDSLVGQALFGDGAAAVIVGSDPDLTKERPLFEMISAAQTILPDSEGAIDGHLREVGLTFHLLKDVPGLISKNIEKALTQAFSPLGISDWNSIFWIAHPGGPAILDQVELKLGLKEEKMRATRHVLSEYGNMSSACVLFIIDEMRKKSAEEGAATTGEGLDWGVLFGFGPGLTVETVVLHSLPTTISVVN

SEQ ID NO: 42 - protein, 397 aa, Actinidia chinensis var. chinensis
MAPSLEEILRAQRSQGPAEILGIGTATPPNCYDQADFPDFYFRVTNSEHMTHLKDKFKQICEKSTVKKRYMYLTEEILKDNPSLCSYMGRSLDVRQNMVMTEVPKLGKEAAAKAIKEWGQPKSKITHLVFCTTSGVDMPGADYHLTKLLGLQPSVKRIMMYQSSCYGGGTGLRLAKDLAENNAGARVLLVCSEISAINFRGPPDTPARLDKLVAQALFGDGAAAVIVGADPDTSIERSLFQLISASQTIVPGSNGGIMGTFGEAGLMCHLIKDVPRLISSNIEKCLMDAFTPIGINDWNSIFWIAHPGGPAILDMVEEKIGLEEEKLRATRHILSEYGNMSSVCVLFILDEMRKKSAEEGKLTTGEGLEWGVLFGFGAGITVETVVLRSMSISNTTH

SEQ ID NO: 43 - protein, 389 aa, Rhododendron dauricum
MVTVEDVRKAQRAEGPATVMAIGTATPPNCVDQSTYPDFYFRITNSEHKAELKEKFQRMCDKSMIKKRYMYLTEEILKENPSVCEYMAPSLDARQDMVVVEVPKLGKEAATKAIKEWGQPKSKITHLVFCTTSGVDMPGADYQLTKLLGLRPSVKRLMMYQQGCFAGGTVLRLAKDLAENNKGARVLVVCSEITAVTFRGPSDTHLDSLVGQALFGDGAAAIIVGADPVPEVEKPLFELVSAAQTILPDSDGAIDGHLREVGLTFHLLKDVPGLISKNIEKALTEAFQPLGISDWNSIFWIAHPGGPAILDQVELKLSLKPEKLRATRHVLSEYGNMSSACVLFILDEMRRKSAEEGLKTTGEGLEWGVLFGFGPGLTVETVVLHSLCT

SEQ ID NO: 44 - protein, 380 aa, Chenopodium quinoa
MASASMNPATILAIGTANPPNVMCQSDYPDYHFRTTNSDHLTDLKAKFKRICDKSMIRKRHFYMNEEILKENPHLGDNNASSIGTRQALCANEIPKLGKEAAEKAIKEWGKPKSMITHLIFGTNSDFDLPGADFRLAKLLGLQPTVKRFILPLGACHAGGTALRIAKDIAENNRGARVLVICSESTAISFHAPSETHLVSLAIFGDGAGAMIVGTDPDEPSERPLFQLVSAGQITLPDSEDGIQARLSEIGMTIHLSPDVPKIIAKNIQTLLSESFDHIGISNWNSIFWVAHPGGPAILDKVEAKLELETSKLSTSRHILSEYGNMWGASVIFVMDEMSKRSLKEGKSTTGEGCEWGVLVAFGPGITVETIVLRSMPINY

SEQ ID NO: 45 - protein, 388 aa, Cajanus cajan
MVSVEDIRKAQRAEGPATVMAIGTATPPNCVDQSTYPDYYFRITNSEHKTELKEKFKRMCDKSMIKKRYMYLNEEILKENPSVCEYMAPSLDARQDMVVVEVPKLGKEAATKAIKEWGQPKSKITHLIFCTTSGVDMPGADYQLTKLLGLRPSVKRYMMYQQGCFAGGTVLRLAKDLAENNKGARVLVVCSEITAVTFRGPSDTHLDSLVGQALFGDGAAAVIVGSDPLPVEKPFFELVWTAQTILPDSEGAIDGHLREVGLTFHLLKDVPGLISKNIEKALVEAFQPLGISDYNSIFWIAHPGGPAILDQVEAKLGLKPEKMEATRHVLSEYGNMSSACVLFILDQMRKKSIENGLGTTGEGLEWGVLFGFGPGLTVETVVLRSVTV

SEQ ID NO: 46 - protein, 393 aa, Lonicera japonica
MGSVTVEEIRKAQRAQGPATVLAIGTATPANCVYQADYPDFYFRITKSEHKAELKEKFKRMCEKSMIRKRYMHLNEEILKENPGICEYMAPSLDARQDMVVVEVPKLGKEAATKAIKEWGQPKSKITHLVFCTTSGVDMPGADYQLTKLLGLRPSVKRLMMYQQGCFAGGTVLRLAKDLAENNAGARVLVVCSEITAVTFRGPSDTHLDSLVGQALFGDGAAAVIIGADPDKSVERPLFELVSAAQTILPDSDGAIDGHLREVGLTFHLLKDVPGLISKNIEKSLKEAFAPIGITDWNSLFWIAHPGGPAILDQVEIKLGLKEEKLRPTRHVLSEYGNMSSACVLFILDELRKKSIEEGKATTGDGLEWGVLFGFGPGLTVETVVLHSVPASI

SEQ ID NO: 47 - protein, 391 aa, Ruta graveolens
MESLKEMRKAQMSEGPAAILAIGTATPNNVYMQADYPDYYFRMTKSEHMTELKDKFRTLCEKSMIRKRHMCFSEEFLKANPEVSKHMGKSLNARQDIAVVETPRLGNEAAVKAIKEWGQPKSSITHLIFCSSAGVDMPGADYQLTRILGLNPSVKRMMVYQQGCYAGGTVLRLAKDLAENNKGSRVLVVCSELTAPTFRGPSPDAVDSLVGQALFADGAAALVVGADPDSSIERALYYLVSASQMLLPDSDGAIEGHIREEGLTVHLKKDVPALFSANIDTPLVEAFKPLGISDWNSIFWIAHPGGPAILDQIEEKLGLKEDKLRASKHVMSEYGNMSSSCVLFVLDEMRSRSLQDGKSTTGEGLDWGVLFGFGPGLTVETVVLRSVPIEA

SEQ ID NO: 48 - protein, 397 aa, Physcomitrella patens subsp. Patens
MASAGDVTRAALPRAQPRAEGPACVLGIGTAVPPAEFLQSEYPDFFFNITNCGEKEALKAKFKRICDKSGIRKRHMFLTEEVLKANPGICTYMEPSLNVRHDIVVVQVPKLAAEAAQKAIKEWGGRKSDITHIVFATTSGVNMPGADHALAKLLGLKPTVKRVMMYQTGCFGGASVLRVAKDLAENNKGARVLAVASEVTAVTYRAPSENHLDGLVGSALFGDGAGVYVVGSDPKPEVEKPLFEVHWAGETILPESDGAIDGHLTEAGLIFHLMKDVPGLISKNIEKFLNEARKPVGSPAWNEMFWAVHPGGPAILDQVEAKLKLTKDKMQGSRDILSEFGNMSSASVLFVLDQIRHRSVKMGASTLGEGSEFGFFIGFGPGLTLEVLVLRAAPNSA

SEQ ID NO: 49 - protein, 387 aa, Rubus idaeus
MGSVAKEAKYPATILAIATANPANCYHQKDYPDFLFRVTKSEDKTELKDKFKRICEKSMVKKRYLGITEESLNANPNICTYKAPSLDSRQDLLVHEVPKLGKEAALKAIEEWGQPISSITHLIFCTASCVDMPGADFQLVKLLGLDPTIKRFMIYQQGCFAGGTVLRIAKDVAENNAGARLLIVCCEITTMFFQQPSENHLDVLVGQALFSDGAAALIVGTNPDPKSERQLFDIMSVRETIIPNSEHGVVAHLREMGFEYYLSSEVPKLVGGKIEEYLNKGFEGIGVDGDWNSLFYSIHPGGPAILNKVEEELGLKEGKLRATRHVLSEFGNMGAPSVLFILDEIRKRSMEEGKATTGEGFEWGVLIGIGPGLTVETVVLRSVSTAN

SEQ ID NO: 50 - protein, 419 aa, Marchantia polymorpha subsp. Ruderalis
MATRVLSSQENFEKLMADLARPNGHVYSQSQSQSGSGQNGAGTSIVAKNTASILAIGKALPPNRICQSTYTDFYFRVTHCSHKTELKNRMQRICDKSGINTRYLLLDEEALKEHSEFYTPGQASIEQRHDLLEEAVPKLAAQAAASALEEWGRPACDVTHLIVVTLSGVAIPGADVRLVKLLGLREDVSRVMLYMLGCYAGVTALRLAKDLAENNPGSRVLIACSEMTATTFRAPSEKSMYDIVGASLFGDGAVGVIVGAKPRPGIERSIFEIHWAGVSLAPDTEHVVQGKLKPDGLYFFLDKSLPGLVGKHIAPFCRSLLDHAPENLNLGFNEVFWAVHPGGPAILNTVEEQLLLNSEKLRASRDVLANYGNVSASSVLYVLDELRHRPGQEEWGAALAFGPGITFEGVLLRRNVNHR

SEQ ID NO: 51 - protein, 388 aa, Oryza sativa
MGKQGGQQLVAAILGIGTAVPPYVLPQSSFPDYYFDISNSNHLLDLKAKFADICEKTMIDKRHVHMSDEFLRSNPSVAAYNSPSINVRQNLTDVTVPQLGAAAARLAIADWGRPACEITHLVMCTTVSGCMPGADFEVVKLLGLPLTTKRCMMYHIGCHGGGTALRLAKDLAENNPGGRVLVVCSEVVSMVFRGPCESHMGNLVGQALFGDAAGAVVVGADPVEANGERTLFEMVSAWQDIIPETEEMVVAKLREEGLVYNLHRDVAARVAASMESLVKKAMVEKDWNEEVFWLVHPGGRDILDRVVLTLGLRDDKVAVCREVMRQHGNTLSSCVIVAMEEMRRRSADRGLSTAGEGLEWGLLFGFGPGLTVETILLRAPPCNQAQAV

SEQ ID NO: 52 - protein, 384 aa, Punica granatum
MGYSQQAKGPATIMAIGTAIPSYVVYQADFPDYYFRLSGCDHMTELKEKFIRICEKSTIRKRHMHLTEEILKQNPAILTYDGPSLNVRQQLVASEVPKLAMEAASKAIEEWGQPVWKITHLVFSSVVGAATPGADYKLIKLLGLEPSVKRVPLYQQGCYVGGTALRIAKDLAENNASARVLVVCVDNTISSFRGPSKHITNLVGQALFSDGASAAIVGADPIPSVERPIFQIAHTSMHLVPDSDSEVTLDFLDAGLIVHVSEKVPSLIADNLEKSLVEALGPTGINDWNSLFWAAHPGGPKILDMIEAKLGLRKEKLRATRTVLREYGNMIGACLLFILDEIRQNSLEAGMATTGEGFDWGILLGFGPGLTVEAVVLRSFPIAK

SEQ ID NO: 53 - protein, 393 aa, Citrus x microcarpa
MAKVKNFLNAKRSKGPASILAIGTANPPTCFNQSDYPDFYFRVTDCEHKTELKDKFKRICDRSAVKKRYLHVTEEVLKENPSMRSYNAPSLDARQALLIEQVPKLGKEAAAKAIKEWGQPLSKITHLVFSAMAGVDIPGADLRLMNLLGLEPSVKRLMIYSQGCFIGGAAIRCAKDFAENNAGARVLVVFSDIMNMYFHEPQEAHLDILVGQAVFGDGAAAVIVGADPEVSIERPLFHVVSSTQMSVPDTNKFIRAHVKEMGMELYLSKDVPATVGKNIEKLLVDAVSPFGISDWNSLFYSVHPGGRAILDQVELNLGLGKEKLRASRHVLSEYGNMGGSSVYFILDEIRKKSMQEAKPTTGDGLEWGVLFAIGPGLTVETVILLSVPIDSAC

SEQ ID NO: 54 - protein, 412 aa, Rhododendron dauricum
MALVNHRENVKGRAQILAIGTANPKNCFRQVDYPDYYFRVTKSDHLIDLKAKFKRMCEKSMIEKRYMHVNEEILEQNPSMNHGGEKMVSSLDVRLDMEIMEIPKLAAEAATKAMDEWGQPKSRITHLVFHSTLGTVMPGVDYELIKLLGLNPSVKRFMLYHLGCYGGGTVLRLAKDLAENNPGSRVLVLCCEMMPSGFHGPPSLQHAHLDILTGHAIFGDGAGAVIVGCVDPSGGTNGVVERGVRRYEQPLFEIHSAYQTVLPDSKDAVGGRLREAGLIYYLSKRLSNDVSGKIDECCLAEAFSAAIKDNFEDWNSLFWIVHPAGRPILDKLDAKLGLNKEKLRASRNVLRDYGNMWSSSVLFVLDEMRKGSIAQRKTTTGEGFEWGVLLGFGPGVTVETVVLRSVPTAKLK

SEQ ID NO: 55 - protein, 389 aa, Curcuma zedoaria
MEANGYRITHSADGPATILAIGTANPTNVVDQNAYPDFYFRVTNSEHLQELKAKFRRICEKAAIRKRHLYLTEEILRENPSLLAPMAPSFDARQAIVVEAVPKLAKEAAEKAIKEWGRPKSDITHLVFCSASGIDMPGSDLQLLKLLGLPPSVNRVMLYNVGCHAGGTALRVAKDLAENNRGARVLAVCSEVTVLSYRGPHPAHIESLFVQALFGDGAAALVVGSDPVDGVERPIFEIASASQVMLPESEEAVGGHLREIGLTFHLKSQLPSIIASNIEQSLTTACSPLGLSDWNQLFWAVHPGGRAILDQVEARLGLEKDRLAATRHVLSEYGNMQSATVLFILDEMRNRSAAEGHATTGEGLDWGVLLGFGPGLSIETVVLHSCRLN

SEQ ID NO: 56 - protein, 391 aa, Garcinia mangostana
MAPAMDSAQNGHQSRGSANVLAIGTANPPNVILQEDYPDFYFKVTNSEHLTDLKEKFKRICVKSKTRKRHFYLTEQILKENPGIATYGAGSLDSRQKILETEIPKLGKEAAMVAIQEWGQPVSKITHVVFATTSGFMMPGADYSITRLLGLNPNVRRVMIYNQGCFAGGTALRVAKDLAENNKGARVLVVCAENTAMTFHGPNENHLDVLVGQAMFSDGAAALIIGANPNLPEERPVYEMVAAHQTIVPESDGAIVAHFYEMGMSYFLKENVIPLFGNNIEACMEAAFKEYGISDWNSLFYSVHPGGRAIVDGIAEKLGLDEENLKATRHVLSEYGNMGSACVIFILDELRKKSKEEKKLTTGDGKEWGCLIGLGPGLTVETVVLRSVPIA

SEQ ID NO: 57 - protein, 391 aa, Arachis hypogaea
MGSLGATQEGNGAKGVATILAIGTANPPNIIRQDDYPDFYFRATKSNHMLHLKEKFQRLCKNSMIEKRHFLYNEDLLMENPNIVTYGASSLNTRQNILIKEVPKLGKEAALKAINEWGQPLSEITHLIFYTTSCFGNMPGPDYHLAKLLGLKPTVNRHMIFNNGCHGGGAVLRVAKDIVENNAGSRVLVVWVETMVASFHGPNPNHMDVLVGQALFGDGAGALIIGTNPKPCIECPLFELVLASQTTIPNTESSINGNIQEMGLVYYLGKEIPIAISENIDKCLINAFRESSVDWNSLFYAIHPGGPSILNRIEEKLGLKKEKLRASRKVLSQYGNMWSPGVIFVLDELRNWSKIEGKSTCGEGKEWGVLVGFGPGLSLELLVLRSFCFDG

SEQ ID NO: 58 - protein, 397 aa, Aquilaria sinensis
MAAQPVEWVRKADRAAGPAAVLAMATANPSNFYLQSDFPDFYFRVTRSDHMSDLKEKFKRICKKTTVRKRHMILTEEILNKNPAIADYWSPSLAARHDLALANIPQLGKEAADKAIKEWGQPKSKITHLVFCTSAGVLMPGADYQLTMLLGLNPSISRLMLHNLGCYAGGTALRVAKDLAENNGGARVLVVCSEANLLNFRGPSETHIDALITQSLFADGAAALIVGSDPDLQTESPLYELISASQRILPESEDAIVGRLTEAGLVPYLPKDIPKLVSTNIRSILEDALAPTGVQDWNSIFWIIHPGMPAILDQTEKLLQLDKEKLKATRHVLSEFGNMFSATVLFILDQLRKGAVAEGKSTTGEGCEWGVLFSFGPGFTVETVLLRSVATATLTDA

SEQ ID NO: 59 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 60 - protein, 333 aa, Stachybotrys bisbyi
MPATRTPIHPEAAAYKNPRYQSGPLSVIPKSFVPYCELMRLELPHGNFLGYFPHLVGLLYGSSASPARLPANEVAFQAVLYIGWTFFMRGAGCAWNDVVDQDFDRKTTRCRVRPVARGAVSTTSANIFGFAMVALAFACISPLPAECQRLGLMTTVLSIIYPFCKRVTNFAQVILGMTLAINFILAAYGAGLPAIEAPYTVPTICVTTAITLLVVFYDVVYARQDTADDLKSGVKGMAVLFRNYVEILLTSITLVIAGLIATTGVLVDNGPYFFVFSVAGLLAALLAMIGGIRYRIFHTWNSYSGWFYALAIFNLLGGYLIEYLDQVPMLNKA

SEQ ID NO: 61 - protein, 329 aa, Stachybotrys chartarum
MSAKVSPMAYTNPRYETGPLSLIPKPIVPYFELMRFELPHGYYLGYFPHLVGIMYGASAGPERLPARDLVFQALLYVGWTFAMRGAGCAWNDNIDQDFDRKTERCRTRPIARGAVSTTAGHVFAVAGVALAFLCLSPLPTECHQLGVLVTVLSVIYPFCKRFTNFAQVILGMTLAANFILAAYGAGLPALEQPYTRPTMSATLAITLLVVFYDVVYARQDTADDLKSGVKGMAVLFRNHIEVLLAVLTCTIGGLLAATGVSVGNGPYYFLFSVAGLTVALLAMIGGIRYRIFHTWNGYSGWFYVLAIINLMSGYFIEYLDNAPILARGS

SEQ ID NO: 62 - protein, 329 aa, Stachybotrys chartarum
MSAKVSPMAYTNPRYERGPLSLIPKPIVPYFELMRFELPHGYYLGYFPHLVGIMYGASAGPERLPARDLVFQALLYVGWTFAMRGAGCAWNDNIDQDFDRKTERCRTRPIARGAVSTTAGHVFAVAGVALAFLCLSPLPTECHQLGVLVTVLSVIYPFCKRFTNFAQVILGMTLAANFILAAYGAGLPALEQPYTRPTMSATLAITLLVVFYDVVYARQDTADDLKSGVKGMAVLFRNHIEVLLAVLTCTIGGLLAATGVSVGNGPYYFLFSVAGLTVALLAMIGGIRYRIFHTWNGYSGWFYVLAIINLMSGYFIEYLDNAPILARGS

SEQ ID NO: 63 - protein, 329 aa, Stachybotrys chlorohalonata
MSPKVSSMPYTNPRYESGPLSLIPKSIVPYFELMRFELPHGYYLGYFPHLVGIMYGASAGPERLPARDLVFQALLYVGWTFAMRGAGCAWNDNIDQDFDRKTERCRTRPIARGAVSTTAGHIFAVAGVALAFLCLSPLPTECHQLGVLVTVLSVIYPFCKRFTNFAQVILGMTLAANFILAAYGAGLPALEQPYTRPTMFATLAITLLVVFYDVVYARQDTADDLKSGVKGMAVLFRNHIEVLLAVLTCTIGGLLAATGVSVGNGPYYFLFSVAGLTVALLAMIGGIRYRIFHTWNGYSGWFYVLAIINLMSGYFIEYLDNAPILARGS

SEQ ID NO: 64 - protein, 322 aa, Cannabis sativa
MAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 65 - protein, 357 aa, Streptomyces actuosus
MTTEVTSFTGAGPHPAASVRRITDDLLQRVEDKLASFLTAERDRYAAMDERALAAVDALTDLVTSGGKRVRPTFCITGYLAAGGDAGDPGIVAAAAGLEMLHVSALIHDDILDNSAQRRGKPTIHTLYGDLHDSHGWRGESRRFGEGIGILIGNLALVYSQELVCQAPPAVLAEWHRLCSEVNIGQCLDVCAAAEFSADPELSRLVALIKSGRYTIHRPLVMGANAASRPDLAAAYVEYGEAVGEAFQLRDDLLDAFGDSTETGKPTGLDFTQHKMTLLLGWAMQRDTHIRTLMTEPGHTPEEVRRRLEDTEVPKDVERHIADLVEQGRAAIADAPIDPQWRQELADMAVRAAYRTN

SEQ ID NO: 66 - protein, 417 aa, Artificial Sequence
  Synthetic Construct
MAFKLAQRLPKSVSSLGSQLSKNAPNQLAAATTSQLINTPGIRHKSRSSAVPSSLSKSMYDHNEEMKAAMKYMDEIYPEVMGQIEKVPQYEEIKPILVRLREAIDYTVPYGKRFKGVHIVSHFKLLADPKFITPENVKLSGVLGWCAEIIQAYFCMLDDIMDDSDTRRGKPTWYKLPGIGLNAVTDVCLMEMFTFELLKRYFPKHPSYADIHEILRNLLFLTHMGQGYDFTFIDPVTRKINFNDFTEENYTKLCRYKIIFSTFHNTLELTSAMANVYDPKKIKQLDPVLMRIGMMHQSQNDFKDLYRDQGEVLKQAEKSVLGTDIKTGQLTWFAQKALSICNDRQRKIIMDNYGKEDNKNSEAVREVYEELDLKGKFMEFEEESFEWLKKEIPKINNGIPHKVFQDYTYGVFKRRPE

SEQ ID NO: 67 - protein, 296 aa, Artificial Sequence
  Synthetic Construct
MAFDFKRYMVEKADSVNKALEAVVQMKEPLKIHESMRYSLLAGGKRVRPMLCIAACELVGGEESTAMPAACAVEMIHTMSLMHDDLPCMDNDDLRRGKPTNHKVFGEDVAVLAGDALLSLAFEHVAVATRGSAPERILRALGQLAKSIGAEGLVAGQVVDICSEGMAEVGLDHLEFIHLHKTAALLQGSVVMGAILGGAKEEEVERLRKFAKCIGLMFQVVDDILDVTKSSHELGKTAGKDLVADKTTYPKLLGVQKSKEFADDLNREAQEQLLHFDSHKAAPLIAIANYIAYRNN

SEQ ID NO: 68 - protein, 271 aa, Artificial Sequence
  Synthetic Construct
MAQNHSYWAAIEADIDTYLKKSIAIRSPETVFEPMHHLTFAAPRTAASAICVAACELVGGERSQAIATASAIHIMHAAAYAHEHLPLTDRPRPNSKPAIQHKYGPNIELLTGDGMASFGFELLAGSIRSDHPNPERILRVIIEISRASGSEGIIDGFYREKEIVDQHSRFDFIEYLCRKKYGEMHACAAASGAILAGGAEEEIQKLRNFGHYAGTLIGLLHKKIDTPQIQNVIGKLKDLALKELEGFHGKNVELLCSLVADASLCEAELEV

SEQ ID NO: 69 - protein, 296 aa, Artificial Sequence
  Synthetic Construct
MAFDFKAYMIGKANSVNKALEDAVLVREPLKIHESMRYSLLAGGKRVRPMLCIAACELFGGTESVAMPSACAVEMIHTMSLMHDDLPCMDNDDLRRGKPTNHKVFGEDVAVLAGDALLAFAFEHIATATKGVSSERIVRVVGELAKCIGSEGLVAGQVVDVCSEGIADVGLEHLEFIHIHKTAALLEGSVVLGAIVGGANDEQISKLRKFARCIGLLFQVVDDILDVTKSSQELGKTAGKDLVADKVTYPKLLGIDKSREFAEKLNREAQEQLAEFDPEKAAPLIALANYIAYRDN

SEQ ID NO: 70 - protein, 263 aa, Artificial Sequence
  Synthetic Construct
MAMKSNSWANIESDIQTHLKKSIPIRAPEDVFEPMHYLTFAAPRTTAPALCIAACEVVGGDGDQAMAAAAAIHLVHAAAYAHENLPLTDRRRPKPPIQHKFNSNIELLTGDGIVPYGFELLAKSMDSNNSDRILRVIIEITQAAGSKGIIDGQFRELDVIDSEINMGLIEYVCKKKEGELNACGAACGAILGGGSEEEIGKLRKFGLYAGMIQGLVHGVGKNREEIQELVRKLRYLAMEELKSLKNRKIDTISSLLETDLCSV

SEQ ID NO: 71 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 72 - protein, 713 aa, Saccharomyces cerevisiae
MSPSAVQSSKLEEQSSEIDKLKAKMSQSASTAQQKKEHEYEHLTSVKIVPQRPISDRLQPAIATHYSPHLDGLQDYQRLHKESIEDPAKFFGSKATQFLNWSKPFDKVFIPDSKTGRPSFQNNAWFLNGQLNACYNCVDRHALKTPNKKAIIFEGDEPGQGYSITYKELLEEVCQVAQVLTYSMGVRKGDTVAVYMPMVPEAIITLLAISRIGAIHSVVFAGFSSNSLRDRINDGDSKVVITTDESNRGGKVIETKRIVDDALRETPGVRHVLVYRKTNNPSVAFHAPRDLDWATEKKKYKTYYPCTPVDSEDPLFLLYTSGSTGAPKGVQHSTAGYLLGALLTMRYTFDTHQEDVFFTAGDIGWITGHTYVVYGPLLYGCATLVFEGTPAYPNYSRYWDIIDEHKVTQFYVAPTALRLLKRAGDSYIENHSLKSLRCLGSVGEPIAAEVWEWYSEKIGKNEIPIVDTYWQTESGSHLVTPLAGGVTPMKPGSASFPFFGIDAVVLDPNTGEELNTSHAEGVLAVKAAWPSFARTIWKNHDRYLDTYLNPYPGYYFTGDGAAKDKDGYIWILGRVDDVVNVSGHRLSTAEIEAAIIEDPIVAECAVVGFNDDLTGQAVAAFVVLKNKSNWSTATDDELQDIKKHLVFTVRKDIGPFAAPKLIILVDDLPKTRSGKIMRRILRKILAGESDQLGDVSTLSNPGIVRHLIDSVKL

SEQ ID NO: 73 - protein, 683 aa, Saccharomyces cerevisiae
MTIKEHKVVYEAHNVKALKAPQHFYNSQPGKGYVTDMQHYQEMYQQSINEPEKFFDKMAKEYLHWDAPYTKVQSGSLNNGDVAWFLNGKLNASYNCVDRHAFANPDKPALIYEADDESDNKIITFGELLRKVSQIAGVLKSWGVKKGDTVAIYLPMIPEAVIAMLAVARIGAIHSVVFAGFSAGSLKDRVVDANSKVVITCDEGKRGGKTINTKKIVDEGLNGVDLVSRILVFQRTGTEGIPMKAGRDYWWHEEAAKQRTYLPPVSCDAEDPLFLLYTSGSTGSPKGVVHTTGGYLLGAALTTRYVFDIHPEDVLFTAGDVGWITGHTYALYGPLTLGTASIIFESTPAYPDYGRYWRIIQRHKATHFYVAPTALRLIKRVGEAEIAKYDTSSLRVLGSVGEPISPDLWEWYHEKVGNKNCVICDTMWQTESGSHLIAPLAGAVPTKPGSATVPFFGINACIIDPVTGVELEGNDVEGVLAVKSPWPSMARSVWNHHDRYMDTYLKPYPGHYFTGDGAGRDHDGYYWIRGRVDDVVNVSGHRLSTSEIEASISNHENVSEAAVVGIPDELTGQTVVAYVSLKDGYLQNNATEGDAEHITPDNLRRELILQVRGEIGPFASPKTIILVRDLPRTRSGKIMRRVLRKVASNEAEQLGDLTTLANPEVVPAIISAVENQFFSQKKK

SEQ ID NO: 74 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 75 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 76 - protein, 329 aa, Streptomyces sp. CL190
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHHLGATGTAAFDVNAVCSGTVFALSSVAGTLVYRGGYALVIGADLYSRILNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAASFALIEW

SEQ ID NO: 77 - protein, 2233 aa, Artificial Sequence
  Synthetic Construct
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLSDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVSVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 78 - DNA, 500 bp, Artificial Sequence
  Synthetic Construct
tggaactttcagtaatacgcttaactgctcattgctatattgaagtacggattagaagccgccgagcgggcgacagccctccgacggaagactctcctccgtgcgtcctggtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactttaacgtcaaggagaaaaaactata

SEQ ID NO: 79 - DNA, 501 bp, Artificial Sequence
  Synthetic Construct
catcgcttcgctgattaattaccccagaaataaggctaaaaaactaatcgcattattatcctatggttgttaatttgattcgttgatttgaaggtttgtggggccaggttactgccaatttttcctcttcataaccataaaagctagtattgtagaatctttattgttcggagcagtgcggcgcgaggcacatctgcgtttcaggaacgcgaccggtgaagaccaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtattgccatgtaatatgattattaaacttctttgcgtccatccaaaaaaaaagtaagaatttttgaaaattcaatataa

SEQ ID NO: 80 - DNA, 509 bp, Artificial Sequence
  Synthetic Construct
ggcttaagtaggttgcaatttctttttctattagtagctaaaaatgggtcacgtgatctatattcgaaaggggcggttgcctcaggaaggcaccggcggtctttcgtccgtgcggagatatctgcgccgttcaggggtccatgtgccttggacgatattaaggcagaaggcagtatcggggcggatcactccgaaccgagattagttaagcccttcccatctcaagatggggagcaaatggcattatactcctgctagaaagttaactgtgcacatattcttaaattatacaatgttctggagagctattgtttaaaaaacaaacatttcgcaggctaaaatgtggagataggattagttttgtagacatatataaacaatcagtaattggattgaaaatttggtgttgtgaattgctcttcattatgcaccttattcaattatcatcaagaatagcaatagttaagtaaacacaagattaacataataaaaaaaataattctttcata

SEQ ID NO: 81 - DNA, 498 bp, Artificial Sequence
  Synthetic Construct
ttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattcttgaaagaatgaaatcgccatgccaagccatcacacggtcttttatgcaattgattgaccgcctgcaacacataggcagtaaaatttttactgaaacgtatataatcatcataagcgacaagtgaggcaacacctttgttaccacattgacaaccccaggtattcatacttcctattagcggaatcaggagtgcaaaaagagaaaataaaagtaaaaaggtagggcaacacatagt

SEQ ID NO: 82 - DNA, 501 bp, Artificial Sequence
  Synthetic Construct
ggacggtagcaacaagaatatagcacgagccgcgaagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaa

SEQ ID NO: 83 - DNA, 489 bp, Artificial Sequence
  Synthetic Construct
gcgacacagagatgacagacggtggcgcaggatccggtttaaacgaggatcccttaagtttaaacaacaacagcaagcaggtgtgcaagacactagagactcctaacatgatgtatgccaataaaacacaagagataaacaacattgcatggaggccccagaggggcgattggtttgggtgcgtgagcggcaagaagtttcaaaacgtccgcgtcctttgagacagcattcgcccagtattttttttattctacaaaccttctataatttcaaagtatttacataattctgtatcagtttaatcaccataatatcgttttctttgtttagtgcaattaatttttcctattgttacttcgggcctttttctgttttatgagctattttttccgtcatccttccccagattttcagcttcatctccagattgtgtctacgtaatgcacgccatcattttaagagaggacagagaagcaagcctcctgaaag

SEQ ID NO: 84 - DNA, 1253 bp, Artificial Sequence
  Synthetic Construct
gatgatggacactagtgtgtcgagaatgtatcaactatatatagtcctaatgccacacaaatatgaagtgggggaagcccattcttaatccggctcaattttggtgcgtgatcgcggcctatgtttgcttccagaaaaagcttagaataatatttctcacctttgatggaatgctcgcgagtgctcgttttgattaccccatatgcattgttgcagcatgcaagcactattgcaagccacgcatggaagaaatttgcaaacacctatagccccgcgttgttgaggaggtggacttggtgtaggaccataaagctgtgcactactatggtgagctctgtcgtctggtgaccttctatctcaggcacatcctcgtttttgtgcatgaggttcgagtcacgcccacggcctattaatccgcgaaataaatgcgaaatctaaattatgacgcaaggctgagagattctgacacgccgcatttgcggggcagtaattatcgggcagttttccggggttcgggatggggtttggagagaaagttcaacacagaccaaaacagcttgggaccacttggatggaggtccccgcagaagagctctggcgcgttggacaaacattgacaatccacggcaaaattgtctacagttccgtgtatgcggatagggatatcttcgggagtatcgcaataggatacaggcactgtgcagattacgcgacatgatagctttgtatgttctacagactctgccgtagcagtctagatataatatcggagttttgtagcgtcgtaaggaaaacttgggttacacaggtttcttgagagccctttgacgttgattgctctggcttccatccaggccctcatgtggttcaggtgcctccgcagtggctggcaagcgtgggggtcaattacgtcacttctattcatgtaccccagactcaattgttgacagcaatttcagcgagaattaaattccacaatcaattctcgctgaaataattaggccgtgatttaattctcgctgaaacagaatcctgtctggggtacagataacaatcaagtaactattatggacgtgcataggaggtggagtccatgacgcaaagggaaatattcattttatcctcgcgaagttgggatgtgtcaaagcgtcgcgctcgctatagtgatgagaatgtctttagtaagcttaagccatataaagaccttccgcctccatatttttttttatccctcttgacaatattaattcctt

SEQ ID NO: 85 - DNA, 1253 bp, Artificial Sequence
  Synthetic Construct
aaggaattaatattgtcaagagggataaaaaaaaatatggaggcggaaggtctttatatggcttaagcttactaaagacattctcatcactatagcgagcgcgacgctttgacacatcccaacttcgcgaggataaaatgaatatttccctttgcgtcatggactccacctcctatgcacgtccataatagttacttgattgttatctgtaccccagacaggattctgtttcagcgagaattaaatcacggcctaattatttcagcgagaattgattgtggaatttaattctcgctgaaattgctgtcaacaattgagtctggggtacatgaatagaagtgacgtaattgacccccacgcttgccagccactgcggaggcacctgaaccacatgagggcctggatggaagccagagcaatcaacgtcaaagggctctcaagaaacctgtgtaacccaagttttccttacgacgctacaaaactccgatattatatctagactgctacggcagagtctgtagaacatacaaagctatcatgtcgcgtaatctgcacagtgcctgtatcctattgcgatactcccgaagatatccctatccgcatacacggaactgtagacaattttgccgtggattgtcaatgtttgtccaacgcgccagagctcttctgcggggacctccatccaagtggtcccaagctgttttggtctgtgttgaactttctctccaaaccccatcccgaaccccggaaaactgcccgataattactgccccgcaaatgcggcgtgtcagaatctctcagccttgcgtcataatttagatttcgcatttatttcgcggattaataggccgtgggcgtgactcgaacctcatgcacaaaaacgaggatgtgcctgagatagaaggtcaccagacgacagagctcaccatagtagtgcacagctttatggtcctacaccaagtccacctcctcaacaacgcggggctataggtgtttgcaaatttcttccatgcgtggcttgcaatagtgcttgcatgctgcaacaatgcatatggggtaatcaaaacgagcactcgcgagcattccatcaaaggtgagaaatattattctaagctttttctggaagcaaacataggccgcgatcacgcaccaaaattgagccggattaagaatgggcttcccccacttcatatttgtgtggcattaggactatatatagttgatacattctcgacacactagtgtccatcatc

SEQ ID NO: 86 - DNA, 665 bp, Artificial Sequence
  Synthetic Construct
gcgcctcaagaaaatgatgctgcaagaagaattgaggaaggaactattcatcttacgttgtttgtatcatcccacgatccaaatcatgttacctacgttaggtacgctaggaactaaaaaaagaaaagaaaagtatgcgttatcactcttcgagccaattcttaattgtgtggggtccgcgaaaatttccggataaatcctgtaaactttaacttaaaccccgtgtttagcgaaattttcaacgaagcgcgcaataaggagaaatattatctaaaagcgagagtttaagcgagttgcaagaatctctacggtacagatgcaacttactatagccaaggtctattcgtattactatggcagcgaaaggagctttaaggttttaattaccccatagccatagattctactcggtctatctatcatgtaacactccgttgatgcgtactagaaaatgacaacgtaccgggcttgagggacatacagagacaattacagtaatcaagagtgtacccaactttaacgaactcagtaaaaaataaggaatgtcgacatcttaattttttatataaagcggtttggtattgattgtttgaagaattttcgggttggtgtttctttctgatgctacatagaagaacatcaaacaactaaaaaaatagtataat

SEQ ID NO: 87 - DNA, 785 bp, Artificial Sequence
  Synthetic Construct
attatactatttttttagttgtttgatgttcttctatgtagcatcagaaagaaacaccaacccgaaaattcttcaaacaatcaataccaaaccgctttatataaaaaattaagatgtcgacattccttattttttactgagttcgttaaagttgggtacactcttgattactgtaattgtctctgtatgtccctcaagcccggtacgttgtcattttctagtacgcatcaacggagtgttacatgatagatagaccgagtagaatctatggctatggggtaattaaaaccttaaagctcctttcgctgccatagtaatacgaatagaccttggctatagtaagttgcatctgtaccgtagagattcttgcaactcgcttaaactctcgcttttagataatatttctccttattgcgcgcttcgttgaaaatttcgctaaacacggggtttaagttaaagtttacaggatttatccggaaattttcgcggaccccacacaattaagaattggctcgaagagtgataacgcatacttttcttttctttttttagttcctagcgtacctaacgtaggtaacatgatttggatcgtgggatgatacaaacaacgtaagatgaatagttccttcctcaattcttcttgcagcatcattttcttgaggcgctctgggcaaggtataaaaagttccattaatacgtctctaaaaaattaaatcatccatctcttaagcagtttttttgataatctcaaatgtacatcagtcaagcgtaactaaattacataa

SEQ ID NO: 88 - DNA, 875 bp, Artificial Sequence
  Synthetic Construct
ttatgtattttagttacgcttgactgatgtacatttgagattatcaaaaaaactgcttaagagatagatggtttaattttttagagacgtattaatggaactttttataccttgcccagagcgcctcaagaaaatgatgctgaaagaagaattgaggaaggaactactcatcttacgttgtttgtatcatcccacgatccaaatcatgttacctacgttaggtacgctaggaactgaaaaaagaaaagaaaagtatgcgttatcactcttcgagccaattcttaattgtgtggggtccgcgaaaacttccggataaatcctgtaaacttaaacttaaaccccgtgtttagcgaaattttcaacgaagcgcgcaataaggagaaatattatataaaagcgagagtttaagcgaggttgcaagaatctctacggtacagatgcaacttactatagccaaggtctattcgtattggtatccaagcagtgaagctactcaggggaaaacatattttcagagatcaaagttatgtcagtctctttttcatgtgtaacttaacgtttgtgcaggtatcataccggcctccacataatttttgtggggaagacgttgttgtagcagtctccttatactctccaacaggtgtttaaagacttcttcaggcctcatagtctacatctggagacaacattagatagaagtttccacagaggcagctttcaatatactttcggctgtgtacatttcatcctgagtgagcgcatattgcataagtactcagtatataaagagacacaatatactccatacttgttgtgagtggttttagcgtattcagtataacaataagaattacatccaagactattaattaact

SEQ ID NO: 89 - DNA, 875 bp, Artificial Sequence
  Synthetic Construct
agttaattaatagtcttggatgtaattcttattgttatactgaatacgctaaaaccactcacaacaagtatggagtatattgtgtctctttatatactgagtacttatgcaatatgcgctcactcaggatgaaatgtacacagccgaaagtatattgaaagctgcctctgtggaaacttctatctaatgttgtctccagatgtagactatgaggcctgaagaagtctttaaacacctgttggagagtataaggagactgctacaacaacgtcttccccacaaaaattatgtggaggccggtatgatacctgcacaaacgttaagttacacatgaaaaagagactgacataactttgatctctgaaaatatgttttcccctgagtagcttcactgcttggataccaatacgaatagaccttggctatagtaagttgcatctgtaccgtagagattcttgcaacctcgcttaaactctcgcttttatataatatttctccttattgcgcgcttcgttgaaaatttcgctaaacacggggtttaagtttaagtttacaggatttatccggaagttttcgcggaccccacacaattaagaattggctcgaagagtgataacgcatacttttcttttcttttttcagttcctagcgtacctaacgtaggtaacatgatttggatcgtgggatgatacaaacaacgtaagatgagtagttccttcctcaattcttctttcagcatcattttcttgaggcgctctgggcaaggtataaaaagttccattaatacgtctctaaaaaattaaaccatctatctcttaagcagtttttttgataatctcaaatgtacatcagtcaagcgtaactaaaatacataa

SEQ ID NO: 90 - protein, 379 aa, Bacillus licheniformis
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYAKGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

(19) United States
(12) Patent Application Publication
(10) Pub. No.: US 2024/0368640 A1
(43) Pub. Date: Nov. 7, 2024

(54) METHODS OF PURIFYING CANNABINOIDS

(71) Applicant: AMYRIS, INC., Emeryville, CA (US)

(72) Inventors: Joshua LENG, Emeryville, CA (US); Paul HILL, Emeryville, CA (US); Rhys DALE, Emeryville, CA (US)

(21) Appl. No.: 18/566,881

(22) PCT Filed: Jun. 3, 2022
(86) PCT No.: PCT/US2022/032230

§ 371 Date: Dec. 4, 2023

Related U.S. Application Data
(60) Provisional application No. 63/196,887, filed on Jun. 4, 2021.
(60) Provisional application No. 63/292,693, filed on Dec. 22, 2021.

(51) Int. Cl. C12P 7/22; C12N 15/52

(52) U.S. Cl. CPC C12P 7/22; C12N 15/52

(57) ABSTRACT

The present disclosure features compositions and methods for producing one or more cannabinoids in a host cell that is genetically modified to express the enzymes of a cannabinoid biosynthetic pathway. Using the compositions and methods of the disclosure, a cannabinoid-producing host cell may be contacted with an oil so as to enhance the recovery of the cannabinoid from the host cell.

### SEQUENCE LISTING

The instant application contains a Sequence Listing which has been submitted electronically in ASCII format and is hereby incorporated by reference in its entirety. Said ASCII copy, created on Jun. 2, 2022, is named 51494-013WO3_Sequence_Listing_6_2_22_ST25 and is 334,532 bytes in size.

### BACKGROUND OF THE INVENTION

Cannabinoids are chemical compounds such as cannabigerols (CBG), cannabichromene (CBC), cannabidiol (CBD), tetrahydrocannabinol (THC), cannabinol (CBN), cannabinodiol (CBDL), cannabicyclol (CBL), cannabielsoin (CBE), cannabitriol (CBT), and tetrahydrocannabinolic acid (THCa), as well as acid forms thereof, which are produced by the cannabis plant. Cannabinoids may be used to improve various aspects of human health. However, producing cannabinoids in preparative amounts and in high yield has been challenging. There remains a need for methods of purifying cannabinoids with high efficiency and high purity.

### SUMMARY OF THE INVENTION

The present disclosure provides methods for producing a cannabinoid in a host cell, such as a yeast cell, and methods for extracting and purifying the cannabinoid from the host cell. For example, using the compositions and methods described herein, a host cell (e.g., yeast cell) may be modified to express one or more enzymes of a cannabinoid biosynthetic pathway, such as an acyl activating enzyme (AAE), a tetraketide synthase (TKS), a cannabigerolic acid synthase (CBGaS), and/or a geranyl pyrophosphate (GPP) synthase. The yeast cell may then be cultured, for example, in the presence of an agent that regulates expression of the one or more enzymes. The yeast cell may be incubated for a time sufficient to allow for biochemical synthesis of a cannabinoid. Purification of the cannabinoid may be accomplished, using the methods herein described, using at least one overlay oil.

In a first aspect, the disclosure features a method of purifying a cannabinoid, the method including culturing a population of host cells that are genetically modified to express one or more enzymes of a cannabinoid biosynthetic pathway in a culture medium and under conditions suitable for the population of host cells to produce the cannabinoid, thereby producing a fermentation composition; contacting the fermentation composition with an oil; and recovering one or more cannabinoids from the fermentation composition and/or the oil.

In another aspect, the disclosure features a method of purifying a cannabinoid, the method including providing a fermentation composition that has been produced by culturing a population of host cells that are genetically modified to express one or more enzymes of a cannabinoid biosynthetic pathway in a culture medium and under conditions suitable for the host cells to produce the cannabinoid; contacting the fermentation composition with an oil; and recovering one or more cannabinoids from the fermentation composition and/or the oil.

In some embodiments, the host cell includes one or more heterologous nucleic acids that each, independently, encode an acyl activating enzyme (AAE), and/or a tetraketide synthase (TKS), and/or a cannabigerolic acid synthase (CBGaS), and/or a geranyl pyrophosphate (GPP) synthase. In some embodiments, the host cell includes heterologous nucleic acids that independently encode an AAE, a TKS, a CBGaS, and a GPP synthase.

In some embodiments, the host cell includes a heterologous nucleic acid that encodes an AAE having an amino acid sequence that is at least 90% (e.g., at least 91%, 92%, 93%, 94%, 95%, 96%, 97%, 98%, or 99%) identical to the amino acid sequence of any one of SEQ ID NO: 1-24. In some embodiments, the AAE has an amino acid sequence that is at least 95% (e.g., at least 96%, 97%, 98%, or 99%) identical to the amino acid sequence of any one of SEQ ID NO: 1-24. In some embodiments, the AAE has the amino acid sequence of any one of SEQ ID NO: 1-24.

In some embodiments, the host cell includes a heterologous nucleic acid that encodes an AAE having an amino acid sequence that is at least 90% (e.g., at least 91%, 92%, 93%, 94%, 95%, 96%, 97%, 98%, or 99%) identical to the amino acid sequence of any one of SEQ ID NO: 1-13. In some embodiments, the AAE has an amino acid sequence that is at least 95% (e.g., at least 96%, 97%, 98%, or 99%) identical to the amino acid sequence of any one of SEQ ID NO: 1-13. In some embodiments, the AAE has the amino acid sequence of any one of SEQ ID NO: 1-13.

In some embodiments, the host cell includes a heterologous nucleic acid that encodes an AAE having an amino acid sequence that is at least 90% (e.g., at least 91%, 92%, 93%, 94%, 95%, 96%, 97%, 98%, or 99%) identical to the amino acid sequence of any one of SEQ ID NO: 1-5. In some embodiments, the AAE has an amino acid sequence that is at least 95% (e.g., at least 96%, 97%, 98%, or 99%) identical to the amino acid sequence of any one of SEQ ID NO: 1-5. In some embodiments, the AAE has the amino acid sequence of any one of SEQ ID NO: 1-5.

In some embodiments, the host cell includes a heterologous nucleic acid that encodes a TKS having an amino acid sequence that is at least 90% (e.g., at least 91%, 92%, 93%, 94%, 95%, 96%, 97%, 98%, or 99%) identical to the amino acid sequence of any one of SEQ ID NO: 25-59. In some embodiments, the TKS has an amino acid sequence that is at least 95% (e.g., at least 96%, 97%, 98%, or 99%) identical to the amino acid sequence of any one of SEQ ID NO: 25-59. In some embodiments, the TKS has the amino acid sequence of any one of SEQ ID NO: 25-59.

In some embodiments, the host cell includes a heterologous nucleic acid that encodes a TKS having an amino acid sequence that is at least 90% (e.g., at least 91%, 92%, 93%, 94%, 95%, 96%, 97%, 98%, or 99%) identical to the amino acid sequence of any one of SEQ ID NO: 25-28. In some embodiments, the TKS has an amino acid sequence that is at least 95% (e.g., at least 96%, 97%, 98%, or 99%) identical to the amino acid sequence of any one of SEQ ID NO: 25-28. In some embodiments, the TKS has the amino acid sequence of any one of SEQ ID NO: 25-28.

In some embodiments, the TKS has an amino acid sequence that is at least 90% (e.g., at least 91%, 92%, 93%, 94%, 95%, 96%, 97%, 98%, or 99%) identical to the amino acid sequence of SEQ ID NO: 25. In some embodiments, the TKS has an amino acid sequence that is at least 95% (e.g., at least 96%, 97%, 98%, or 99%) identical to the amino acid sequence of SEQ ID NO: 25. In some embodiments, the TKS has the amino acid sequence of SEQ ID NO: 25.

In some embodiments, the host cell includes a heterologous nucleic acid that encodes a CBGaS having an amino acid sequence that is at least 90% (e.g., at least 91%, 92%, 93%, 94%, 95%, 96%, 97%, 98%, or 99%) identical to the amino acid sequence of any one of SEQ ID NO: 60-64. In some embodiments, the CBGaS has an amino acid sequence that is at least 95% (e.g., at least 96%, 97%, 98%, or 99%) identical to the amino acid sequence of any one of SEQ ID NO: 60-64. In some embodiments, the CBGaS has the amino acid sequence of any one of SEQ ID NO: 60-64.

In some embodiments, the host cell includes a heterologous nucleic acid that encodes a GPP synthase having an amino acid sequence that is at least 90% (e.g., at least 91%, 92%, 93%, 94%, 95%, 96%, 97%, 98%, or 99%) identical to the amino acid sequence of any one of SEQ ID NO: 65-70. In some embodiments, the GPP synthase has an amino acid sequence that is at least 95% (e.g., at least 96%, 97%, 98%, or 99%) identical to the amino acid sequence of any one of SEQ ID NO: 65-70. In some embodiments, the GPP synthase has the amino acid sequence of any one of SEQ ID NO: 65-70.

In some embodiments, the GPP synthase has an amino acid sequence that is at least 90% (e.g., at least 91%, 92%, 93%, 94%, 95%, 96%, 97%, 98%, or 99%) identical to the amino acid sequence of SEQ ID NO: 65. In some embodiments, the GPP synthase has an amino acid sequence that is at least 95% (e.g., at least 96%, 97%, 98%, or 99%) identical to the amino acid sequence of SEQ ID NO: 65. In some embodiments, the GPP synthase has the amino acid sequence of SEQ ID NO: 65.

In some embodiments, the host cell includes heterologous nucleic acids that independently encode an AAE having the amino acid sequence of any one of SEQ ID NO: 1-24, a TKS having the amino acid sequence of any one of SEQ ID NO: 25-59, a CBGaS having the amino acid sequences of any one of SEQ ID NO: 60-64, and a GPP synthase having the amino acid sequence of any one of SEQ ID NO: 65-70.

In some embodiments, the host cell further includes one or more heterologous nucleic acids that each, independently, encode an enzyme of the mevalonate biosynthetic pathway, wherein the enzyme is selected from an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase. In some embodiments, the host cell includes heterologous nucleic acids that independently encode an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

In some embodiments, the host cell further includes a heterologous nucleic acid that encodes an olivetolic acid cyclase (OAC). In some embodiments, the OAC has an amino acid sequence that is at least 90% (e.g., at least 91%, 92%, 93%, 94%, 95%, 96%, 97%, 98%, or 99%) identical to the amino acid sequence of SEQ ID NO: 71. In some embodiments, the OAC has an amino acid sequence that is at least 95% (e.g., at least 96%, 97%, 98%, or 99%) identical to the amino acid sequence of SEQ ID NO: 71. In some embodiments, the OAC has the amino acid sequence of SEQ ID NO: 71.

In some embodiments, the host cell further includes one or more heterologous nucleic acids that each, independently, encode an acetyl-CoA synthase, and/or an aldehyde dehydrogenase, and/or a pyruvate decarboxylase. In some embodiments, the acetyl-CoA synthase has an amino acid sequence that is at least 90% (e.g., at least 91%, 92%, 93%, 94%, 95%, 96%, 97%, 98%, or 99%) identical to the amino acid sequence of SEQ ID NO: 72. In some embodiments, the acetyl-CoA synthase has an amino acid sequence that is at least 95% (e.g., at least 96%, 97%, 98%, or 99%) identical to the amino acid sequence of SEQ ID NO: 72. In some embodiments, the acetyl-CoA synthase has the amino acid sequence of SEQ ID NO: 72.

In some embodiments, the acetyl-CoA synthase has an amino acid sequence that is at least 90% (e.g., at least 91%, 92%, 93%, 94%, 95%, 96%, 97%, 98%, or 99%) identical to the amino acid sequence of SEQ ID NO: 73. In some embodiments, the acetyl-CoA synthase has an amino acid sequence that is at least 95% (e.g., at least 96%, 97%, 98%, or 99%) identical to the amino acid sequence of SEQ ID NO: 73. In some embodiments, the acetyl-CoA synthase has the amino acid sequence of SEQ ID NO: 73.

In some embodiments, the aldehyde dehydrogenase has an amino acid sequence that is at least 90% (e.g., at least 91%, 92%, 93%, 94%, 95%, 96%, 97%, 98%, or 99%) identical to the amino acid sequence of SEQ ID NO: 74. In some embodiments, the aldehyde dehydrogenase has an amino acid sequence that is at least 95% (e.g., at least 96%, 97%, 98%, or 99%) identical to the amino acid sequence of SEQ ID NO: 74. In some embodiments, the aldehyde dehydrogenase synthase has the amino acid sequence of SEQ ID NO: 74.

In some embodiments, the pyruvate decarboxylase has an amino acid sequence that is at least 90% (e.g., at least 91%, 92%, 93%, 94%, 95%, 96%, 97%, 98%, or 99%) identical to the amino acid sequence of SEQ ID NO: 75. In some embodiments, the pyruvate decarboxylase has an amino acid sequence that is at least 95% (e.g., at least 96%, 97%, 98%, or 99%) identical to the amino acid sequence of SEQ ID NO: 75. In some embodiments, the pyruvate decarboxylase has the amino acid sequence of SEQ ID NO: 75.

In some embodiments, the host cell contains a heterologous nucleic acid encoding an aceto-CoA carboxylase (ACC). In some embodiments, the heterologous nucleic acid encodes a ACC having an amino acid sequence that is at least 90% identical to the amino acid sequence of SEQ ID NO: 77 (e.g., an amino acid sequence that is 90%, 91%, 92%, 93%, 94%, 95%, 96%, 97%, 98%, 99%, or 100% identical to SEQ ID NO: 77). In some embodiments, the ACC has an amino acid sequence that is at least 95% identical to the amino acid sequence of SEQ ID NO: 77 (e.g., an amino acid sequence that is 95%, 96%, 97%, 98%, 99%, or 100% identical to SEQ ID NO: 77). In some embodiments, the ACC has the amino acid sequence of SEQ ID NO: 77.

In some embodiments, the host cell contains a heterologous nucleic acid encoding an ACC and an acetoacetyl-CoA synthase (AACS) instead of a heterologous nucleic acid encoding an acetyl-CoA thiolase. In some embodiments, the heterologous nucleic acid encodes an ACC having an amino acid sequence that is at least 90% identical to the amino acid sequence of SEQ ID NO: 77 (e.g., an amino acid sequence that is 90%, 91%, 92%, 93%, 94%, 95%, 96%, 97%, 98%, 99%, or 100% identical to SEQ ID NO: 77). In some embodiments, the ACC has an amino acid sequence that is at least 95% identical to the amino acid sequence of SEQ ID NO: 77 (e.g., an amino acid sequence that is 95%, 96%, 97%, 98%, 99%, or 100% identical to SEQ ID NO: 77). In some embodiments, the ACC has the amino acid sequence of SEQ ID NO: 77. In some embodiments, the heterologous nucleic acid encodes an AACS having an amino acid sequence that is at least 90% identical to the amino acid sequence of SEQ ID NO: 76 (e.g., an amino acid sequence that is 90%, 91%, 92%, 93%, 94%, 95%, 96%, 97%, 98%, 99%, or 100% identical to SEQ ID NO: 76). In some embodiments, the AACS has an amino acid sequence that is at least 95% identical to the amino acid sequence of SEQ ID NO: 76 (e.g., an amino acid sequence that is 95%, 96%, 97%, 98%, 99%, or 100% identical to SEQ ID NO: 76). In some embodiments, the AACS has the amino acid sequence of SEQ ID NO: 76.

In some embodiments, expression of the one or more heterologous nucleic acids is regulated by an exogenous agent. In some embodiments, the exogenous agent comprises a regulator of gene expression. In some embodiments, the exogenous agent decreases production of the cannabinoid. In some embodiments, the exogenous agent is maltose. In some embodiments, the exogenous agent increases production of the cannabinoid. In some embodiments, the exogenous agent is galactose. In some embodiments, the exogenous agent is galactose and expression of one or more heterologous nucleic acids encoding the AAE, TKS, and CBGaS enzymes is under the control of a GAL promoter. In some embodiments, expression of one or more heterologous nucleic acids encoding the AAE, TKS, and CBGaS enzymes is under the control of a galactose-responsive promoter, a maltose-responsive promoter, or a combination of both.

In some embodiments, the invention includes culturing the host cell with the precursor required to make the cannabinoid. In some embodiments, the precursor required to make the cannabinoid is hexanoate. In some embodiments, the cannabinoid is cannabidiolic acid (CBDA), cannabidiol (CBD), cannabigerolic acid (CBGA), cannabigerol (CBG), tetrahydrocannabinol (THC), or tetrahydrocannabinolic acid (THCa). In some embodiments, the host cell is a yeast cell or yeast strain. In some embodiments, the yeast cell is S. cerevisiae. In some embodiments, the fermentation composition is separated into a supernatant and a pellet by solid liquid centrifugation. In some embodiments, the fermentation composition is contacted with the oil after the fermentation is adjusted to a pH of about 8.

In some embodiments, the oil is added to the fermentation composition at a concentration of from about 1% to about 25% w/w. In some embodiments, the oil is added to the fermentation at a concentration of about 10% w/w. In some embodiments, the fermentation composition is mixed with the oil for a duration of from about 10 minutes to about 120 minutes. In some embodiments the fermentation composition is mixed with the oil for about 60 minutes. In some embodiments, the fermentation composition is maintained at a temperature of 55° C. In some embodiments, the fermentation composition is subsequently mixed with the oil for an additional period of time with a duration of from about 1 minute to about 600 minutes. In some embodiments, the fermentation composition is maintained at a temperature of 70° C. In some embodiments, the fermentation composition undergoes a plurality of liquid centrifugation steps after being contacted with the oil. In some embodiments, the fermentation composition undergoes a plurality of demulsification steps between each of the plurality of liquid centrifugation steps.

In some embodiments, the cannabinoid is recovered with a purity of between 50% w/w and 100% w/w. In some embodiments, the recovered cannabinoid has a purity of between 70% and 100%. In some embodiments, the recovered cannabinoid has a purity of between 80% and 100%. In some embodiments, the recovered cannabinoid has a purity of between 90% and 100%. In some embodiments, the recovered cannabinoid has a purity of about 80%, 81%, 82%, 83%, 84%, 85%, 86%, 87%, 88%, 89%, 90%, 91%, 92%, 93%, 94%, 95%, 96%, 97%, 98%, 99%, or 100% w/w. In some embodiments, the cannabinoid is recovered with one or more impurities. In some embodiments, the impurities are present in an amount of from about 0.1% to about 1% w/w. In some embodiments, the impurities are present in an amount of from about 0.1% to about 0.6% w/w. In some embodiments, the impurities are present in an amount of about 0.1%, 0.2%, 0.3%, 0.4%, 0.5%, 0.6%, 0.7%, 0.8%, 0.9%, or 1% w/w. In some embodiments, the one or more impurities include one or more of cannabidivarinic acid, cannabidivarin, cannabidiolic acid, SCBGa, cannabigerol, tetrahydrocannabivarinic acid, cannabinol, cannabinolic acid, delta-9-tetrahydrocannabinol, delta-8-tetrahydrocannabinol, cannabicyclol, cannabichromene, tetrahydrocannabinolic acid, cannabichromenic acid, and cannabidiol. In some embodiments, the molar yield of the cannabinoid is between 60% and 100%. In some embodiments, the molar yield is between 90% and 100%.

In some embodiments, the oil includes a mineral oil, a vegetable oil, a synthetic ester, or a fatty alcohol. In some embodiments, the oil includes a vegetable oil. In some embodiments, the oil is soybean oil, sunflower oil, safflower oil, canola oil, grapeseed oil, or castor oil. In some embodiments, the synthetic ester is ESTEREX™ A51. In some embodiments, the fatty alcohol is oleyl alcohol or JARCOL™ I-16. In some embodiments, the oil suppresses a pungent smell of the exogenous agent.

In another aspect, the disclosure features a cannabinoid produced using any of the above-described methods. In some embodiments, the cannabinoid is recovered with a purity of between 50% w/w and 100% w/w. In some embodiments, the recovered cannabinoid has a purity of between 70% and 100%. In some embodiments, the recovered cannabinoid has a purity of between 80% and 100%. In some embodiments, the recovered cannabinoid has a purity of between 90% and 100%. In some embodiments, the recovered cannabinoid has a purity of about 80%, 81%, 82%, 83%, 84%, 85%, 86%, 87%, 88%, 89%, 90%, 91%, 92%, 93%, 94%, 95%, 96%, 97%, 98%, 99%, or 100% w/w. In some embodiments, the cannabinoid is recovered with one or more impurities. In some embodiments, the impurities are present in an amount of from about 0.1% to about 1% w/w. In some embodiments, the impurities are present in an amount of from about 0.1% to about 0.6% w/w. In some embodiments, the impurities are present in an amount of about 0.1%, 0.2%, 0.3%, 0.4%, 0.5%, 0.6%, 0.7%, 0.8%, 0.9%, or 1% w/w. In some embodiments, the one or more impurities include one or more of cannabidivarinic acid, cannabidivarin, cannabidiolic acid, SCBGa, cannabigerol, tetrahydrocannabivarinic acid, cannabinol, cannabinolic acid, delta-9-tetrahydrocannabinol, delta-8-tetrahydrocannabinol, cannabicyclol, cannabichromene, tetrahydrocannabinolic acid, cannabichromenic acid, and cannabidiol. In some embodiments, the molar yield of the cannabinoid is between 60% and 100%. In some embodiments, the molar yield is between 90% and 100%.

In another aspect, the disclosure features a composition including cannabigerol (CBG), wherein the CBG is produced by a method including: a) culturing a population of host cells that are genetically modified to express one or more enzymes of a CBG biosynthetic pathway in a culture medium and under conditions suitable for the population of host cells to produce CBG thereby producing a fermentation composition; and b) recovering CBG from the fermentation composition.

In some embodiments, the CBG is present in the composition with a purity of between 50% w/w and 100% w/w. In some embodiments, the recovered CBG has a purity of between 70% and 100%. In some embodiments, the recovered CBG has a purity of between 80% and 100%. In some embodiments, the recovered CBG has a purity of between 90% and 100%. In some embodiments, the recovered CBG has a purity of about 80%, 81%, 82%, 83%, 84%, 85%, 86%, 87%, 88%, 89%, 90%, 91%, 92%, 93%, 94%, 95%, 96%, 97%, 98%, 99%, or 100% w/w. In some embodiments, the CBG is recovered with one or more impurities. In some embodiments, the impurities are present in an amount of from about 0.1% to about 1% w/w. In some embodiments, the impurities are present in an amount of from about 0.1% to about 0.6% w/w. In some embodiments, the impurities are present in an amount of about 0.1%, 0.2%, 0.3%, 0.4%, 0.5%, 0.6%, 0.7%, 0.8%, 0.9%, or 1% w/w. In some embodiments, the one or more impurities include one or more of cannabidivarinic acid, cannabidivarin, cannabidiolic acid, SCBGa, cannabigerol, tetrahydrocannabivarinic acid, cannabinol, cannabinolic acid, delta-9-tetrahydrocannabinol, delta-8-tetrahydrocannabinol, cannabicyclol, cannabichromene, tetrahydrocannabinolic acid, cannabichromenic acid, and cannabidiol.

In another aspect, the disclosure features a composition including CBG and one or more impurities that include a compound selected from cannabidivarinic acid, cannabidivarin, cannabidiolic acid, SCBGa, cannabigerol, tetrahydrocannabivarinic acid, cannabinol, cannabinolic acid, delta-9-tetrahydrocannabinol, delta-8-tetrahydrocannabinol, cannabicyclol, cannabichromene, tetrahydrocannabinolic acid, cannabichromenic acid, or cannabidiol. In some embodiments, the CBG has a purity of between 70% and 100%. In some embodiments, the CBG has a purity of between 80% and 100%. In some embodiments, the CBG has a purity of between 90% and 100%. In some embodiments, the CBG has a purity of about 80%, 81%, 82%, 83%, 84%, 85%, 86%, 87%, 88%, 89%, 90%, 91%, 92%, 93%, 94%, 95%, 96%, 97%, 98%, 99%, or 100% w/w. In some embodiments, the impurities are present in an amount of from about 0.1% to about 1% w/w. In some embodiments, the impurities are present in an amount of from about 0.1% to about 0.6% w/w. In some embodiments, the impurities are present in an amount of about 0.1%, 0.2%, 0.3%, 0.4%, 0.5%, 0.6%, 0.7%, 0.8%, 0.9%, or 1% w/W.

In another aspect, the disclosure features a mixture including the population of a genetically modified host cell herein described and a culture medium. In some embodiments, the culture medium comprises an exogenous agent and an oil. In some embodiments, the exogenous agent is hexanoate. In some embodiments, the oil is soybean oil, sunflower oil, safflower oil, castor oil, ESTEREX™ A51, or JARCOL™ I-16.

### DEFINITIONS

As used herein the singular forms “a,” “an,” and, “the” include plural reference unless the context clearly dictates otherwise.

The term “about” when modifying a numerical value or range herein includes normal variation encountered in the field, and includes plus or minus 1-10% (e.g., 1%, 2%, 3%, 4%, 5%, 6%, 7%, 8%, 9% or 10%) of the numerical value or end points of the numerical range. Thus, a value of 10 includes all numerical values from 9 to 11. All numerical ranges described herein include the endpoints of the range unless otherwise noted, and all numerical values in-between the end points, to the first significant digit.

As used herein, the term “cannabinoid” refers to a chemical substance that binds or interacts with a cannabinoid receptor (for example, a human cannabinoid receptor) and includes, without limitation, chemical compounds such endocannabinoids, phytocannabinoids, and synthetic cannabinoids. Synthetic compounds are chemicals made to mimic phytocannabinoids which are naturally found in the cannabis plant (e.g., Cannabis sativa), including but not limited to cannabigerols (CBG), cannabichromene (CBC), cannabidiol (CBD), tetrahydrocannabinol (THC), cannabinol (CBN), cannabinodiol (CBDL), cannabicyclol (CBL), cannabielsoin (CBE), and cannabitriol (CBT).

As used herein, the term “capable of producing” refers to a host cell which is genetically modified to include the enzymes necessary for the production of a given compound in accordance with a biochemical pathway that produces the compound. For example, a cell (e.g., a yeast cell) “capable of producing” a cannabinoid is one that contains the enzymes necessary for production of the cannabinoid according to the cannabinoid biosynthetic pathway.

As used herein, the term “exogenous” refers a substance or compound that originated outside an organism or cell. The exogenous substance or compound can retain its normal function or activity when introduced into an organism or host cell described herein.

As used herein, the term “fermentation composition” refers to a composition which contains genetically modified host cells and products or metabolites produced by the genetically modified host cells. An example of a fermentation composition is a whole cell broth, which may be the entire contents of a vessel, including cells, aqueous phase, and compounds produced from the genetically modified host cells.

As used herein, the term “gene” refers to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, gRNA, or micro RNA.

A “genetic pathway” or “biosynthetic pathway” as used herein refer to a set of at least two different coding sequences, where the coding sequences encode enzymes that catalyze different parts of a synthetic pathway to form a desired product (e.g., a cannabinoid). In a genetic pathway a first encoded enzyme uses a substrate to make a first product which in turn is used as a substrate for a second encoded enzyme to make a second product. In some embodiments, the genetic pathway includes 3 or more members (e.g., 3, 4, 5, 6, 7, 8, 9, etc.), wherein the product of one encoded enzyme is the substrate for the next enzyme in the synthetic pathway.

As used herein, the term “genetic switch” refers to one or more genetic elements that allow controlled expression of enzymes, e.g., enzymes that catalyze the reactions of cannabinoid biosynthesis pathways. For example, a genetic switch can include one or more promoters operably linked to one or more genes encoding a biosynthetic enzyme, or one or more promoters operably linked to a transcriptional regulator which regulates expression one or more biosynthetic enzymes.

As used herein, the term “genetically modified” denotes a host cell that contains a heterologous nucleotide sequence. The genetically modified host cells described herein typically do not exist in nature.

As used herein, the term “heterologous” refers to what is not normally found in nature. The term “heterologous compound” refers to the production of a compound by a cell that does not normally produce the compound, or to the production of a compound at a level not normally produced by the cell. For example, a cannabinoid can be a heterologous compound.

A “heterologous genetic pathway” or a “heterologous biosynthetic pathway” as used herein refer to a genetic pathway that does not normally or naturally exist in an organism or cell.

The term “host cell” as used in the context of this invention refers to a microorganism, such as yeast, and includes an individual cell or cell culture contains a heterologous vector or heterologous polynucleotide as described herein. Host cells include progeny of a single host cell, and the progeny may not necessarily be completely identical (in morphology or in total DNA complement) to the original parent cell due to natural, accidental, or deliberate mutation and/or change. A host cell includes cells into which a recombinant vector or a heterologous polynucleotide of the invention has been introduced, including by transformation, transfection, and the like.

As used herein, the term “medium” refers to culture medium and/or fermentation medium.

The terms “modified,” “recombinant” and “engineered,” when used to modify a host cell described herein, refer to host cells or organisms that do not exist in nature, or express compounds, nucleic acids or proteins at levels that are not expressed by naturally occurring cells or organisms.

As used herein, the terms “oil,” “overlay oil,” or “overlay” refer to a biologically compatible hydrophobic, lipophilic, carbon-containing substance including but not limited to geologically-derived crude oil, distillate fractions of geologically-derived crude oil, vegetable oil, algal oil, microbial lipids, or synthetic oils. The oil is neither itself toxic to a biological molecule, a cell, a tissue, or a subject, nor does it degrade (if the oil degrades) at a rate that produces byproducts at toxic concentrations to a biological molecule, a cell, a tissue or a subject. Preferred examples of oils include but are not limited to avocado oil, canola oil, grapeseed oil, hemp oil, soybean oil, jojoba oil, and sunflower oil.

As used herein, the phrase “operably linked” refers to a functional linkage between nucleic acid sequences such that the linked promoter and/or regulatory region functionally controls expression of the coding sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid

The terms “polynucleotide” and “nucleic acid” are used interchangeably and refer to a single or double-stranded polymer of deoxyribonucleotide or ribonucleotide bases read from the 5′ to the 3′ end. A nucleic acid as used in the present invention will generally contain phosphodiester bonds, although in some cases, nucleic acid analogs may be used that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); positive backbones; non-ionic backbones, and non-ribose backbones. Nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus, the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo-and ribo-nucleotides, and combinations of bases, including uracil, adenine, thymine, cytosine, guanine, inosine, xanthine hypoxanthine, isocytosine, isoguanine, etc. Nucleic acid sequences are presented in the 5′ to 3′ direction unless otherwise specified.

As used herein, the terms “polypeptide,” “peptide,” and “protein” are used interchangeably to refer to a polymer of amino acid residues. The terms encompass amino acid chains of any length, including full-length proteins, wherein the amino acid residues are linked by covalent peptide bonds.

As used herein, the term “production” generally refers to an amount of compound produced by a genetically modified host cell provided herein. In some embodiments, production is expressed as a yield of the compound by the host cell. In other embodiments, production is expressed as a productivity of the host cell in producing the compound.

As used herein, the term “productivity” refers to production of a compound by a host cell, expressed as the amount of non-catabolic compound produced (by weight) per amount of fermentation broth in which the host cell is cultured (by volume) over time (per hour).

As used herein, the term “promoter” refers to a synthetic or naturally derived nucleic acid that is capable of activating, increasing or enhancing expression of a DNA coding sequence, or inactivating, decreasing, or inhibiting expression of a DNA coding sequence. A promoter may contain one or more specific transcriptional regulatory sequences to further enhance or repress expression and/or to alter the spatial expression and/or temporal expression of the coding sequence. A promoter may be positioned 5′ (upstream) of the coding sequence under its control. A promoter may also initiate transcription in the downstream (3′) direction, the upstream (5′) direction, or be designed to initiate transcription in both the downstream (3′) and upstream (5′) directions. The distance between the promoter and a coding sequence to be expressed may be approximately the same as the distance between that promoter and the native nucleic acid sequence it controls. As is known in the art, variation in this distance may be accommodated without loss of promoter function. The term also includes a regulated promoter, which generally allows transcription of the nucleic acid sequence while in a permissive environment (e.g., microaerobic fermentation conditions, or the presence of maltose), but ceases transcription of the nucleic acid sequence while in a non-permissive environment (e.g., aerobic fermentation conditions, or in the absence of maltose). Promoters used herein can be constitutive, inducible, or repressible.

The term “yield” refers to production of a compound by a host cell, expressed as the amount of compound produced per amount of carbon source consumed by the host cell, by weight.

### BRIEF DESCRIPTION OF THE DRAWINGS

FIG. 1 is a graph showing the optical density of wild-type yeast cell cultures (strain Y46850) in a flask experiment (SF1) testing the impact of castor oil, control (no oil), JARCOL™ I-16, safflower oil, soybean oil, or sunflower oil on the growth of the yeast cells. Optical density values for castor oil, control (no oil), JARCOL™ I-16, safflower oil, soybean, and sunflower oil respectively are shown below each label along the horizontal axis.

FIG. 2 is a graph showing the optical density of wild-type yeast cell cultures (strain Y46850) in a flask experiment (SF2) testing the impact of castor oil, control (no oil), JARCOL™ I-16, safflower oil, soybean oil, or sunflower oil on the growth of the yeast cells. Growth was not significantly different when wild-type yeast cells were cultured in the oils compared to control.

FIG. 3 is a graph showing the optical density of wild-type yeast cell cultures (strain Y46850) in a flask experiment (SF3) testing the impact of castor oil, control (no oil), JARCOL™ I-16, safflower oil, soybean oil, or sunflower oil on the growth of the yeast cells. Growth was not significantly different when wild-type yeast cells were cultured in the oils compared to control.

FIG. 4 is a graph showing the distillation yield of recovered CBD in a fermentation experiment using wild-type yeast (strain Y46850). Approximately 50 g/L of CBD from an external source was spiked into the fermentation compositions. The yield of recovered CBD was measured following distillation using a wiped film evaporator in pure (100%) oil solutions and in fermentation compositions containing 10% sunflower oil or soybean oil.

FIG. 5 is a graph showing the distillate purity of recovered CBD in a fermentation experiment using wild-type yeast (strain Y46850). Approximately 50 g/L of CBD from an external source was spiked into the fermentation compositions. The purity of recovered CBD was measured following distillation using a wiped film evaporator in pure (100%) oil solutions and in fermentation compositions containing 10% sunflower oil or soybean oil.

FIG. 6 is a graph showing the boiling point of JARCOL™ I-12 and the simulated boiling points of JARCOL™ I-16, IPM, ethyl myristate, ethyl palmitate, ethyl oleate, CBD, avocado oil, canola oil, grapeseed oil, hemp oil, soybean oil, jojoba oil, and sunflower oil shown as a range of temperatures.

FIG. 7 is a graph showing the viscosities of avocado oil, canola oil, grapeseed oil, hemp oil, jojoba oil, soybean oil, and sunflower oil as a function of temperature.

FIG. 8 is a graph showing the viscosities of castor oil, avocado oil, canola oil, grapeseed oil, hemp oil, jojoba oil, soybean oil, and sunflower oil as a function of temperature.

FIG. 9 is an image showing the visual appearance of CBDA (as hemp oil) in various oils. Solubility of CBDA was qualitatively characterized in formulations containing DURASYN® 164/Hemp oil 10:1 v/v (vial #1), DRAKEOL® 19/Hemp oil 10:1 v/v (vial #2), JARCOL™ |-16/Hemp oil 10:1 v/v (vial #3), castor oil/Hemp oil 10:1 v/v (vial #4), and soybean oil/Hemp oil 10:1 v/v (vial #5). Formulations containing JARCOL™ |-16, castor oil, or soybean oil appeared clear indicating good solubility whereas formulations containing DRAKEOL® 19 or DURASYN® 164 showed precipitates indicating lower solubility.

FIG. 10 is a graph showing the amount (in grams) of CBGA recovered from CBGA-producing yeast cells (strain Y61508) after two separate fractionation experiments (8904-10 and 8904-9). Most of the CBGA was extracted into the oil (blue) fraction compared to the amount remaining in the aqueous (green) and pellet (pink) fractions.

FIG. 11 is a graph showing the amount (in milligrams) of CBGA recovered from CBGA-producing yeast cells (strain Y62456) at varying timepoints (˜72 minutes, ˜96 minutes, and ˜118 minutes) with no oil present in the composition. CBGA was consistently found in the broth (pink) fraction compared to the amount remaining in the pellet (blue) fraction.

FIG. 12 is a graph showing the accumulation of CBDA, in the broth fraction (pink) or pellet fraction (green), over time when produced by yeast cells (strain Y64604). Timepoints represent the culture time prior to fractionation.

FIG. 13 is a graph showing the amount of CBDA in the broth fraction (pink), oil fraction (blue), pellet fraction (green), or supernatant fraction (yellow) after extraction with 10% soybean oil. CBDA was produced by yeast cells (strain Y64604).

FIG. 14A is a graph showing the effects of feeding CBGA-producing yeast cells (strain Y61508) with hexanoate at various days of cell culture. Yeast were fed with secondary feed of 10% w/w hexanoate in soybean oil, 15% w/w hexanoate in soybean oil, or control with no secondary feed and only primary feed of 10 g/L hexanoate in sucrose. Performance was similar with secondary feeding compared to the control group. Results are unadjusted to lot variability of sucrose.

FIG. 14B is a graph showing the effect of feeding CBGA-producing yeast cells (strain Y61508) with hexanoate on the average productivity (grams of CBGA produced/liter of cell culture medium/hour) of the cells at various days of cell culture. Yeast were fed with secondary feed of 10% w/w hexanoate in soybean oil, 15% w/w hexanoate in soybean oil, or control with no secondary feed and only primary feed of 10 g/L hexanoate in sucrose. The average productivity was higher with secondary feeding of 15% w/w hexanoate in soybean oil compared to the control group. The average productivity was lower with secondary feeding of 10% w/w hexanoate in soybean oil compared to the control group. Results are unadjusted to lot variability of sucrose.

FIG. 14C is a graph showing the effect of feeding CBGA-producing yeast cells (strain Y61508) with hexanoate on the average yield of CVGA production at various days of cell culture. Yeast were fed with secondary feed of 10% w/w hexanoate in soybean oil, 15% w/w hexanoate in soybean oil, or control with no secondary feed and only primary feed of 10 g/L hexanoate in sucrose. The average yield was higher with secondary feeding of 15% w/w hexanoate in soybean oil compared to the control group. The average yield was lower with secondary feeding of 10% w/w hexanoate in soybean oil compared to the control group. Results are unadjusted to lot variability of sucrose.

### DETAILED DESCRIPTION OF THE INVENTION

The present disclosure provides methods for purifying a cannabinoid from a fermentation composition. For example, using the compositions and methods described herein, a cannabinoid may be purified from a fermentation composition produced by culturing host cells genetically modified to express one or more enzyme of a cannabinoid biosynthetic pathways in a culture medium. To facilitate recovery of the cannabinoid from the fermentation composition, the fermentation composition may be contacted with an oil overlay. Exemplary oils that may be used in conjunction with the compositions and methods of the disclosure include, without limitation, soybean oil, sunflower oil, safflower oil, castor oil, ESTEREX™ A51, and JARCOL™ I-16.

The sections that follow provide a description of the host cells, heterologous cannabinoid expression systems, and oil overlays that may be used to produce and recover a cannabinoid.

### Methods of Purifying a Cannabinoid

In an aspect, the disclosure provides methods for purifying a cannabinoid from a fermentation composition. In some embodiments, the method uses a solvent of high boiling point and/or high flash point (e.g., an oil, e.g., a mineral oil, a vegetable oil, a synthetic ester, or a fatty alcohol) in the fermentation composition. In some embodiments, the solvent is a vegetable oil (e.g., soybean oil, sunflower oil, safflower oil, castor oil), a synthetic ester (e.g., ESTEREX™ A51), or a fatty alcohol (e.g., oleyl alcohol or JARCOL™ I-16).

In some embodiments, the concentration of oil in the fermentation composition is between 1% w/w and 50% w/w (e.g., 1.1% w/w, 1.2% w/w, 1.3% w/w, 1.4% w/w, 1.5% w/w, 1.6% w/w, 1.7% w/W, 1.8% w/w, 1.9% w/w, 2% w/w, 3% w/w, 4% w/w, 5% w/w, 6% w/w, 7% w/w, 8% w/w, 9% w/w, 10% w/W, 11% w/w, 12% w/w, 13% w/w, 14% w/w, 15% w/w, 16% w/w, 17% w/w, 18% w/w, 19% w/w, 20% w/W, 21% w/w, 22% w/w, 23% w/w, 24% w/w, 25% w/w, 26% w/w, 27% w/w, 28% w/w, 29% w/w, 30% w/w, 31% w/w, 32% w/w, 33% w/w, 34% w/w, 35% w/w, 36% w/w, 37% w/w, 38% w/w, 39% w/w, 40% w/W, 41% w/w, 42% w/w, 43% w/w, 44% w/w, 45% w/w, 46% w/w, 47% w/w, 48% w/w, 49% w/w, or 50% w/w).

In some embodiments, the fermentation occurs in the presence of the oil. In some embodiments, the oil is present in the fermentation composition at the beginning of the fermentation reaction and is present until the fermentation reaches completion. In some embodiments, the fermentation is allowed to reach completion prior to the addition of the oil. In some embodiments, the oil is added into the fermentation composition after the fermentation is completed and the oil is mixed with the fermentation composition for 1 min to 600 min (e.g., 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, 60 min, 61 min, 62 min, 63 min, 64 min, 65 min, 66 min, 67 min, 68 min, 69 min, 70 min, 71 min, 72 min, 73 min, 74 min, 75 min, 76 min, 77 min, 78 min, 79 min, 80 min, 81 min, 82 min, 83 min, 84 min, 85 min, 86 min, 87 min, 88 min, 89 min, 90 min, 91 min, 92 min, 93 min, 94 min, 95 min, 96 min, 97 min, 98 min, 99 min, 100 min, 101 min, 102 min, 103 min, 104 min, 105 min, 106 min, 107 min, 108 min, 109 min, 110 min, 111 min, 112 min, 113 min, 114 min, 115 min, 116 min, 117 min, 118 min, 119 min, 120 min, 121 min, 122 min, 123 min, 124 min, 125 min, 126 min, 127 min, 128 min, 129 min, 130 min, 131 min, 132 min, 133 min, 134 min, 135 min, 136 min, 137 min, 138 min, 139 min, 140 min, 151 min, 152 min, 153 min, 154 min, 155 min, 156 min, 157 min, 158 min, 159 min, 160 min, 161 min, 162 min, 163 min, 164 min, 165 min, 166 min, 167 min, 168 min, 169 min, 170 min, 171 min, 172 min, 173 min, 174 min, 175 min, 176 min, 177 min, 178 min, 179 min, 180 min, 181 min, 182 min, 183 min, 184 min, 185 min, 186 min, 187 min, 188 min, 189 min, 190 min, 191 min, 192 min, 193 min, 194 min, 195 min, 196 min, 197 min, 198 min, 199 min, 200 min, 201 min, 202 min, 203 min, 204 min, 205 min, 206 min, 207 min, 208 min, 209 min, 210 min, 211 min, 212 min, 213 min, 214 min, 215 min, 216 min, 217 min, 218 min, 219 min, 220 min, 221 min, 222 min, 223 min, 224 min, 225 min, 226 min, 227 min, 228 min, 229 min, 230 min, 231 min, 232 min, 233 min, 234 min, 235 min, 236 min, 237 min, 238 min, 239 min, 240 min, 241 min, 242 min, 243 min, 244 min, 245 min, 246 min, 247 min, 248 min, 249 min, 250 min, 251 min, 252 min, 253 min, 254 min, 255 min, 256 min, 257 min, 258 min, 259 min, 260 min, 261 min, 262 min, 263 min, 264 min, 265 min, 266 min, 267 min, 268 min, 269 min, 270 min, 271 min, 272 min, 273 min, 274 min, 275 min, 276 min, 277 min, 278 min, 279 min, 280 min, 281 min, 282 min, 283 min, 284 min, 285 min, 286 min, 287 min, 288 min, 289 min, 290 min, 291 min, 292 min, 293 min, 294 min, 295 min, 296 min, 297 min, 298 min, 299 min, 300 min, 301 min, 302 min, 303 min, 304 min, 305 min, 306 min, 307 min, 308 min, 309 min, 310 min, 311 min, 312 min, 313 min, 314 min, 315 min, 316 min, 317 min, 318 min, 319 min, 320 min, 321 min, 322 min, 323 min, 324 min, 325 min, 326 min, 327 min, 328 min, 329 min, 330 min, 331 min, 332 min, 333 min, 334 min, 335 min, 336 min, 337 min, 338 min, 339 min, 340 min, 341 min, 342 min, 343 min, 344 min, 345 min, 346 min, 347 min, 348 min, 349 min, 350 min, 351 min, 352 min, 353 min, 354 min, 355 min, 356 min, 357 min, 358 min, 359 min, 360 min, 361 min, 362 min, 363 min, 364 min, 365 min, 366 min, 367 min, 368 min, 369 min, 370 min, 371 min, 372 min, 373 min, 374 min, 375 min, 376 min, 377 min, 378 min, 379 min, 380 min, 381 min, 382 min, 383 min, 384 min, 385 min, 386 min, 387 min, 388 min, 389 min, 390 min, 391 min, 392 min, 393 min, 394 min, 395 min, 396 min, 397 min, 398 min, 399 min, 400 min, 401 min, 402 min, 403 min, 404 min, 405 min, 406 min, 407 min, 408 min, 409 min, 410 min, 411 min, 412 min, 413 min, 414 min, 415 min, 416 min, 417 min, 418 min, 419 min, 420 min, 421 min, 422 min, 423 min, 424 min, 425 min, 426 min, 427 min, 428 min, 429 min, 430 min, 431 min, 432 min, 433 min, 434 min, 435 min, 436 min, 437 min, 438 min, 439 min, 440 min, 441 min, 442 min, 443 min, 444 min, 445 min, 446 min, 447 min, 448 min, 449 min, 450 min, 451 min, 452 min, 453 min, 454 min, 455 min, 456 min, 457 min, 458 min, 459 min, 460 min, 461 min, 462 min, 463 min, 464 min, 465 min, 466 min, 467 min, 468 min, 469 min, 470 min, 471 min, 472 min, 473 min, 474 min, 475 min, 476 min, 477 min, 478 min, 479 min, 480 min, 481 min, 482 min, 483 min, 484 min, 485 min, 486 min, 487 min, 488 min, 489 min, 490 min, 491 min, 492 min, 493 min, 494 min, 495 min, 496 min, 497 min, 498 min, 499 min, 500 min, 501 min, 502 min, 503 min, 504 min, 505 min, 506 min, 507 min, 508 min, 509 min, 510 min, 511 min, 512 min, 513 min, 514 min, 515 min, 516 min, 517 min, 518 min, 519 min, 520 min, 521 min, 522 min, 523 min, 524 min, 525 min, 526 min, 527 min, 528 min, 529 min, 530 min, 531 min, 532 min, 533 min, 534 min, 535 min, 536 min, 537 min, 538 min, 539 min, 540 min, 541 min, 542 min, 543 min, 544 min, 545 min, 546 min, 547 min, 548 min, 549 min, 550 min, 551 min, 552 min, 553 min, 554 min, 555 min, 556 min, 557 min, 558 min, 559 min, 560 min, 561 min, 562 min, 563 min, 564 min, 565 min, 566 min, 567 min, 568 min, 569 min, 570 min, 571 min, 572 min, 573 min, 574 min, 575 min, 576 min, 577 min, 578 min, 579 min, 580 min, 581 min, 582 min, 583 min, 584 min, 585 min, 586 min, 587 min, 588 min, 589 min, 590 min, 591 min, 592 min, 593 min, 594 min, 595 min, 596 min, 597 min, 598 min, 599 min, or 600 min).

In some embodiments, the population of host cells, the fermentation composition, and the oil are separated into an aqueous liquid fraction, an oily liquid fraction, and a pellet by way of centrifugation. In some embodiments, the oil fraction is recovered from the liquid fraction following centrifugation. In some embodiments, the oil fraction undergoes centrifugation more than one time (e.g., 2 times, 3 times, 4 times, 5 times, 6 times, 7 times, 8 times, 9 times, or 10 times) to ensure separation from the aqueous liquid fraction. In some embodiments, the fermentation composition may be mixed for a time and at a temperature sufficient to allow for demulsification of the fermentation composition before the cannabinoid undergoes decarboxylation, and the cannabinoid may be recovered. In some embodiments, at least one demulsification step (e.g., 2 steps, 3 steps, 4 steps, 5 steps, 6 steps, 7 steps, 8 steps, 9 steps, or 10 steps) is performed between centrifugation steps.

Demulsification may, in some embodiments, be conducted using a demulsification aid, such as an enzymatic composition including a serine protease (e.g., TERGAZYME®). In some embodiments, the enzymatic composition includes between 0.003% and 20% serine protease by weight (e.g., between 0.003% and 15%, between 0.005% and 10%, between 0.007% and 7%, and between 0.01% and 5% serine protease by weight). In some embodiments, the enzymatic composition includes between 0.01% and 10% serine protease by weight (e.g., between 0.01% and 10%, between 0.02% and 9%, between 0.03% and 8%, between 0.04% and 7%, between 0.05% and 6%, between 0.06% and 5%, between 0.07% and 4%, between 0.08% and 3%, between 0.08% and 2%, between 0.09% and 1%, and between 0.1% and 1% serine protease by weight). In some embodiments, the enzymatic composition includes between 0.01% and 5% by serine protease by weight (e.g., between 0.01% and 5%, between 0.05% and 4%, between 0.1% and 3%, between 0.5% and 2% serine protease by weight).

In some embodiments, the serine protease is a subtilisin. In some embodiments, the subtilisin is from Bacillus licheniformis. In some embodiments, the subtilisin is subtilisin Carlsberg. In some embodiments, the subtilisin has an amino acid sequence that is at least 90% (e.g., at least 91%, 92%, 93%, 94%, 95%, 96%, 97%, 98%, or 99%) identical to the amino acid sequence of SEQ ID NO: 90. In some embodiments, the subtilisin has an amino acid sequence that is at least 95% (e.g., at least 96%, 97%, 98%, or 99%) identical to the amino acid sequence of SEQ ID NO: 90. In some embodiments, the subtilisin has the amino acid sequence of SEQ ID NO: 90.

In some embodiments, the serine protease is deactivated by exposure to 300 ppm hypochlorite at a temperature of 85° F. for less than one minute; 3.5 ppm hypochlorite at a temperature of 100° F. for 2 min; a pH below 4 for 30 min at a temperature of 140° F.; or by heating to a temperature of 175 OF for 10 min.

In some embodiments, the enzymatic composition includes an alkylaryl sulfonate salt. In some embodiments, the alkylaryl sulfonate includes a linear alkylaryl sulfonate salt. In some embodiments, the enzymatic composition includes a phosphate salt. In some embodiments, the enzymatic composition includes a carbonate salt. In some embodiments, the salt is a sodium salt.

In some embodiments, the enzymatic composition has a pH of between 8.5 and 11 (e.g., between pH 8.7 and pH 10.5, between pH 9.0 and pH 10, and between pH 9.2 and pH 9.7) in a 1% (w/v) solution. In some embodiments, the enzymatic composition has a pH of about 9.5 in a 1% (w/v) solution. In some embodiments, the oil contains the cannabinoid and the oil undergoes further purification using distillation (e.g., using an evaporator). In some embodiments, distillation is performed to evaporate the oil solvent to recover crystalized cannabinoid. In some embodiments, distillation is performed more than one time (e.g., 2 times, 3 times, 4 times, 5 times, 6 times, 7 times, 8 times, 9 times, or 10 times) to improve purity. In some embodiments, the recovered cannabinoid has a purity between 50% and 100% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 89%, 90%, 91%, 92%, 93%, 94%, 95%, 96%, 97%, 98%, 99%, or 100%). In some embodiments, the cannabinoid is recovered with one or more impurities. In some embodiments, the impurities are present in an amount of about 0.1%, 0.2%, 0.3%, 0.4%, 0.5%, 0.6%, 0.7%, 0.8%, 0.9%, or 1% w/w. In some embodiments, the impurity is present in an amount of about 0.1% w/w. In some embodiments, the impurity is present in an amount of about 0.3% w/w. In some embodiments, the impurity is present in an amount of about 0.6% w/w. In some embodiments, the one or more impurities include one or more of cannabidivarinic acid, cannabidivarin, cannabidiolic acid, SCBGa, cannabigerol, tetrahydrocannabivarinic acid, cannabinol, cannabinolic acid, delta-9-tetrahydrocannabinol, delta-8-tetrahydrocannabinol, cannabicyclol, cannabichromene, tetrahydrocannabinolic acid, cannabichromenic acid, and cannabidiol.

In some embodiments, the molar yield of the cannabinoid is between 60% and 100% (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 89%, 90%, 91%, 92%, 93%, 94%, 95%, 96%, 97%, 98%, 99%, or 100%).

### Cannabinoid Biosynthetic Pathway

In an aspect, a host cell described herein includes one or more nucleic acids encoding one or more enzymes of a heterologous genetic pathway that produces a cannabinoid or a precursor of a cannabinoid. The cannabinoid biosynthetic pathway may begin with hexanoic acid as the substrate for an acyl activating enzyme (AAE) to produce hexanoyl-CoA, which is used as the substrate of a tetraketide synthase (TKS) to produce tetraketide-CoA, which is used by an olivetolic acid cyclase (OAC) to produce olivetolic acid, which is then used to produce a cannabigerolic acid by a geranyl pyrophosphate (GPP) synthase and a cannabigerolic acid synthase (CBGaS). In some embodiments, the cannabinoid precursor that is produced is a substrate in the cannabinoid pathway (e.g., hexanoate or olivetolic acid). In some embodiments, the precursor is a substrate for an AAE, a TKS, an OAC, a CBGaS, or a GPP synthase. In some embodiments, the precursor, substrate, or intermediate in the cannabinoid pathway is hexanoate, olivetol, or olivetolic acid. In some embodiments, the precursor is hexanoate. In some embodiments, the host cell does not contain the precursor, substrate or intermediate in an amount sufficient to produce the cannabinoid or a precursor of the cannabinoid. In some embodiments, the host cell does not contain hexanoate at a level or in an amount sufficient to produce the cannabinoid in an amount over 10 mg/L. In some embodiments, the heterologous genetic pathway encodes at least one enzyme selected from the group consisting of an AAE, a TKS, an OAC, a CBGaS, or a GPP synthase. In some embodiments, the genetically modified host cell includes an AAE, TKS, OAC, CBGaS, and a GPP synthase. The cannabinoid pathway is described in Keasling et al. (U.S. Pat. No. 10,563,211), the disclosure of which is incorporated herein by reference.

### Acyl Activating Enzymes

Some embodiments concern a host cell that includes a heterologous AAE such that the host cell is capable of producing a cannabinoid. The AAE may be from Cannabis sativa or may be an enzyme from another plant or fungal source which has been shown to have AAE activity in the cannabinoid biosynthetic pathway, resulting in the production of the cannabinoid precursor olivetolic acid. In some embodiments, the host cell contains a heterologous nucleic acid that encodes an AAE having an amino acid sequence that is at least 90% identical to the amino acid sequence of any one of SEQ ID NO: 1-24 (e.g., an amino acid sequence that is 90%, 91%, 92%, 93%, 94%, 95%, 96%, 97%, 98%, 99%, or 100% identical to any one of SEQ ID NO: 1-24). In some embodiments, the AAE has an amino acid sequence that is at least 95% identical to the amino acid sequence of any one of SEQ ID NO: 1-24 (e.g., an amino acid sequence that is 95%, 96%, 97%, 98%, 99%, or 100% identical to any one of SEQ ID NO: 1-24). In some embodiments, the AAE has the amino acid sequence of any one of SEQ ID NO: 1-24.

In some embodiments, the host cell contains a heterologous nucleic acid that encodes an AAE having an amino acid sequence that is at least 90% identical to the amino acid sequence of any one of SEQ ID NO: 1-13 (e.g., an amino acid sequence that is 90%, 91%, 92%, 93%, 94%, 95%, 96%, 97%, 98%, 99%, or 100% identical to any one of SEQ ID NO: 1-13). In some embodiments, the AAE has an amino acid sequence that is at least 95% identical to the amino acid sequence of any one of SEQ ID NO: 1-13 (e.g., an amino acid sequence that is 95%, 96%, 97%, 98%, 99%, or 100% identical to any one of SEQ ID NO: 1-13). In some embodiments, the AAE has the amino acid sequence of any one of SEQ ID NO: 1-13.

In some embodiments, the host cell contains a heterologous nucleic acid that encodes an AAE having an amino acid sequence that is at least 90% identical to the amino acid sequence of any one of SEQ ID NO: 1-5 (e.g., an amino acid sequence that is 90%, 91%, 92%, 93%, 94%, 95%, 96%, 97%, 98%, 99%, or 100% identical to any one of SEQ ID NO: 1-5). In some embodiments, the AAE has an amino acid sequence that is at least 95% identical to the amino acid sequence of any one of SEQ ID NO: 1-5 (e.g., an amino acid sequence that is 95%, 96%, 97%, 98%, 99%, or 100% identical to any one of SEQ ID NO: 1-5). In some embodiments, the AAE has the amino acid sequence of any one of SEQ ID NO: 1-5.

### Tetraketide Synthase Enzymes

Some embodiments concern a host cell that includes a heterologous TKS such that the host cell is capable of producing a cannabinoid. A TKS uses the hexanoyl-CoA precursor to generate tetraketide-CoA. The TKS may be from Cannabis sativa or may be an enzyme from another plant or fungal source which has been shown to have TKS activity in the cannabinoid biosynthetic pathway, resulting in the production of the cannabinoid precursor olivetolic acid. In some embodiments, the host cell contains a heterologous nucleic acid that encodes a TKS having an amino acid sequence that is at least 90% identical to the amino acid sequence of any one of SEQ ID NO: 25-59 (e.g., an amino acid sequence that is 90%, 91%, 92%, 93%, 94%, 95%, 96%, 97%, 98%, 99%, or 100% identical to any one of SEQ ID NO: 25-59). In some embodiments, the TKS has an amino acid sequence that is at least 95% identical to the amino acid sequence of any one of SEQ ID NO: 25-59 (e.g., an amino acid sequence that is 95%, 96%, 97%, 98%, 99%, or 100% identical to any one of SEQ ID NO: 25-59). In some embodiments, the TKS has the amino acid sequence of any one of SEQ ID NO: 25-59.

In some embodiments, the host cell contains a heterologous nucleic acid that encodes a TKS having an amino acid sequence that is at least 90% identical to the amino acid sequence of any one of SEQ ID NO: 25-28 (e.g., an amino acid sequence that is 90%, 91%, 92%, 93%, 94%, 95%, 96%, 97%, 98%, 99%, or 100% identical to any one of SEQ ID NO: 25-28). In some embodiments, the TKS has an amino acid sequence that is at least 95% identical to the amino acid sequence of any one of SEQ ID NO: 25-28 (e.g., an amino acid sequence that is 95%, 96%, 97%, 98%, 99%, or 100% identical to any one of SEQ ID NO: 25-28). In some embodiments, the TKS has the amino acid sequence of any one of SEQ ID NO: 25-28.

In some embodiments, the host cell contains a heterologous nucleic acid that encodes a TKS having an amino acid sequence that is at least 90% identical to the amino acid sequence of SEQ ID NO: 25 (e.g., an amino acid sequence that is 90%, 91%, 92%, 93%, 94%, 95%, 96%, 97%, 98%, 99%, or 100% identical to SEQ ID NO: 25). In some embodiments, the TKS has an amino acid sequence that is at least 95% identical to the amino acid sequence of SEQ ID NO: 25 (e.g., an amino acid sequence that is 95%, 96%, 97%, 98%, 99%, or 100% identical to SEQ ID NO: 25). In some embodiments, the TKS has the amino acid sequence of SEQ ID NO: 25.

### Cannabigerolic Acid Synthases

Some embodiments concern a host cell that includes a heterologous CBGaS such that the host cell is capable of producing a cannabinoid. A CBGaS uses the olivetolic acid precursor and GPP precursor to generate cannabigerolic acid. The CBGaS may be from Cannabis sativa or may be an enzyme from another plant or fungal source which has been shown to have CBGaS activity in the cannabinoid biosynthetic pathway, resulting in the production of the cannabinoid cannabigerolic acid. In some embodiments, the host cell contains a heterologous nucleic acid that encodes a CBGaS having an amino acid sequence that is at least 90% identical to the amino acid sequence of any one of SEQ ID NO: 60-64 (e.g., an amino acid sequence that is 90%, 91%, 92%, 93%, 94%, 95%, 96%, 97%, 98%, 99%, or 100% identical to any one of SEQ ID NO: 60-64). In some embodiments, the CBGaS has an amino acid sequence that is at least 95% identical to the amino acid sequence of any one of SEQ ID NO: 60-64 (e.g., an amino acid sequence that is 95%, 96%, 97%, 98%, 99%, or 100% identical to any one of SEQ ID NO: 60-64). In some embodiments, the CBGaS has the amino acid sequence of any one of SEQ ID NO: 60-64.

### Geranyl Pyrophosphate Synthase

Some embodiments concern a host cell that includes a heterologous GPP synthase such that the host cell is capable of producing a cannabinoid. A GPP synthase uses the product of the isoprenoid biosynthesis pathway precursor to generate cannabigerolic acid together with a prenyltransferase enzyme. The GPP synthase may be from Cannabis sativa or may be an enzyme from another plant or bacterial source which has been shown to have GPP synthase activity in the cannabinoid biosynthetic pathway, resulting in the production of the cannabinoid cannabigerolic acid.

In some embodiments, the host cell contains a heterologous nucleic acid that encodes a GPP synthase having an amino acid sequence that is at least 90% identical to the amino acid sequence of any one of SEQ ID NO: 65-70 (e.g., an amino acid sequence that is 90%, 91%, 92%, 93%, 94%, 95%, 96%, 97%, 98%, 99%, or 100% identical to any one of SEQ ID NO: 65-70). In some embodiments, the GPP synthase has an amino acid sequence that is at least 95% identical to the amino acid sequence of any one of SEQ ID NO: 65-70 (e.g., an amino acid sequence that is 95%, 96%, 97%, 98%, 99%, or 100% identical to any one of SEQ ID NO: 65-70). In some embodiments, the GPP synthase has the amino acid sequence of any one of SEQ ID NO: 65-70.

In some embodiments, the host cell contains a heterologous nucleic acid that encodes a GPP synthase having an amino acid sequence that is at least 90% identical to the amino acid sequence of SEQ ID NO: 65 (e.g., an amino acid sequence that is 90%, 91%, 92%, 93%, 94%, 95%, 96%, 97%, 98%, 99%, or 100% identical to SEQ ID NO: 65). In some embodiments, the GPP synthase has an amino acid sequence that is at least 95% identical to the amino acid sequence of SEQ ID NO: 65 (e.g., an amino acid sequence that is 95%, 96%, 97%, 98%, 99%, or 100% identical to SEQ ID NO: 65). In some embodiments, the GPP synthase has the amino acid sequence of SEQ ID NO: 65.

### Additional Enzymes

The host cell may further express other heterologous enzymes in addition to the AAE, TKS, CBGaS, and/or GPP synthase. For example, the host cell may include an olivetolic acid cyclase (OAC) as part of the cannabinoid biosynthetic pathway. The OAC may have an amino acid sequence that is at least 90% (e.g., at least 91%, 92%, 93%, 94%, 95%, 96%, 97%, 98%, or 99%) identical to SEQ ID NO: 71. In some embodiments, the OAC has an amino acid sequence of SEQ ID NO: 71. In some embodiments, the host cell may include a heterologous nucleic acid that encodes at least one enzyme from the mevalonate biosynthetic pathway. Enzymes which make up the mevalonate biosynthetic pathway may include but are not limited to an acetyl-CoA thiolase, a HMG-COA synthase, a HMG-COA reductase, a mevalonate kinase, a phosphomevalonate kinase, a mevalonate pyrophosphate decarboxylase, and an IPP:DMAPP isomerase. In some embodiments, the host cell includes a heterologous nucleic acid that encodes the acetyl-CoA thiolase, the HMG-COA synthase, the HMG-COA reductase, the mevalonate kinase, the phosphomevalonate kinase, the mevalonate pyrophosphate decarboxylase, and the IPP:DMAPP isomerase of the mevalonate biosynthesis pathway.

In some embodiments, the host cell may express heterologous enzymes of the central carbon metabolism. Enzymes of the central carbon metabolism may include an acetyl-CoA synthase, an aldehyde dehydrogenase, and a pyruvate decarboxylase. In some embodiments, the host cell includes heterologous nucleic acids that independently encode an acetyl-CoA synthase, and/or an aldehyde dehydrogenase, and/or a pyruvate decarboxylase. In some embodiments, the acetyl-CoA synthase and the aldehyde dehydrogenase from Saccharomyces cerevisiae, and the pyruvate decarboxylase from Zymomonas mobilis. In some embodiments, the acetyl-CoA synthase has an amino acid sequence that is at least 90% (e.g., at least 91%, 92%, 93%, 94%, 95%, 96%, 97%, 98%, or 99%) identical to the amino acid sequence of SEQ ID NO: 72. In some embodiments, the acetyl-CoA synthase has the amino acid sequence of SEQ ID NO: 72. In some embodiments, the host cell expresses a heterologous acetyl-CoA synthase having an amino acid sequence that is at least 90% (e.g., at least 91%, 92%, 93%, 94%, 95%, 96%, 97%, 98%, or 99%) identical to the amino acid sequence of SEQ ID NO: 73. In some embodiments, the acetyl-CoA synthase has the amino acid sequence of SEQ ID NO: 73. In some embodiments, the aldehyde dehydrogenase has an amino acid sequence that is at least 90% (e.g., at least 91%, 92%, 93%, 94%, 95%, 96%, 97%, 98%, or 99%) identical to the amino acid sequence of SEQ ID NO: 74. In some embodiments, the aldehyde dehydrogenase has the amino acid sequence of SEQ ID NO: 74. In some embodiments, the pyruvate dehydrogenase has an amino acid sequence that is at least 90% (e.g., at least 91%, 92%, 93%, 94%, 95%, 96%, 97%, 98%, or 99%) identical to the amino acid sequence of SEQ ID NO: 75. In some embodiments, the pyruvate decarboxylase has an amino acid sequence of SEQ ID NO: 75.

Due to the inherent degeneracy of the genetic code, other polynucleotides which encode substantially the same or functionally equivalent polypeptides can also be used to clone and express the polynucleotides encoding the protein components of the heterologous genetic pathway described herein.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24:216-8).

Those of skill in the art will recognize that, due to the degenerate nature of the genetic code, a variety of DNA molecules differing in their nucleotide sequences can be used to encode a given enzyme of the disclosure. Any one of the polypeptide sequences disclosed herein may be encoded by DNA molecules of any sequence that encode the amino acid sequences of the polypeptides and proteins of the enzymes utilized in the methods of the disclosure. In a similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for the compositions and methods provided herein are encompassed by the disclosure. In some embodiments, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (e.g., Pearson W. R., 1994, Methods in Mol Biol 25:365-89).

The following six groups each contain amino acids that are conservative substitutions for one another: 1) Serine(S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. A typical algorithm used for comparing a molecule sequence to a database containing a large number of sequences from different organisms is the computer program BLAST. When searching a database containing sequences from a large number of different organisms, it is typical to compare amino acid sequences.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in a host cell, for example, a yeast.

In addition, genes encoding these enzymes can be identified from other fungal and bacterial species and can be expressed in the host cell. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorphs, Candida spp., Trichosporon spp., Yamadazyma spp., including Y. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia coli, Zymomonas mobilis, Staphylococcus aureus, Bacillus spp., Clostridium spp., Corynebacterium spp., Pseudomonas spp., Lactococcus spp., Enterobacter spp., and Salmonella spp.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous ADA genes, proteins, or enzymes, techniques may include, but are not limited to, cloning a gene by PCR using primers based on a published sequence of an ADA gene/enzyme or by degenerate PCR using degenerate primers designed to amplify a conserved region among ADA genes.

Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K., Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, JGI Phyzome v12.1, BLAST, NCBI RefSeq, UniProt KB, or MetaCYC Protein annotations in the UniProt Knowledgebase may also be used to identify enzymes which have a similar function in addition to the National Center for Biotechnology Information RefSeq database. The candidate gene or enzyme may be identified within the above-mentioned databases in accordance with the teachings herein.

### Modified Host Cells

In one aspect, provided herein are host cells comprising at least one enzyme of the cannabinoid biosynthetic pathway. In some embodiments, the cannabinoid biosynthetic pathway contains a genetic regulatory element, such as a nucleic acid sequence, that is regulated by an exogenous agent. In some embodiments, the exogenous agent acts to regulate expression of the heterologous genetic pathway. Thus, in some embodiments, the exogenous agent can be a regulator of gene expression.

In some embodiments, the exogenous agent can be used as a carbon source by the host cell. For example, the same exogenous agent can both regulate production of a cannabinoid and provide a carbon source for growth of the host cell. In some embodiments, the exogenous agent is galactose. In some embodiments, the exogenous agent is maltose.

In some embodiments, the genetic regulatory element is a nucleic acid sequence, such as a promoter.

In some embodiments, the genetic regulatory element is a galactose-responsive promoter. In some embodiments, galactose positively regulates expression of the cannabinoid biosynthetic pathway, thereby increasing production of the cannabinoid. In some embodiments, the galactose-responsive promoter is a GAL1 promoter. In some embodiments, the galactose-responsive promoter is a GAL10 promoter. In some embodiments, the galactose-responsive promoter is a GAL2, GAL3, or GAL7 promoter. In some embodiments, heterologous genetic pathway contains the galactose-responsive regulatory elements described in Westfall et al. (PNAS (2012) vol. 109: E111-118). In some embodiments, the host cell lacks the gal1 gene and is unable to metabolize galactose, but galactose can still induce galactose-regulated genes.

TABLE 1
Exemplary GAL Promoter Sequences

| Promoter | Sequence |
|---|---|
| pGAL1 | SEQ ID NO: 78 |
| pGAL10 | SEQ ID NO: 79 |
| pGAL2 | SEQ ID NO: 80 |
| pGAL3 | SEQ ID NO: 81 |
| pGAL7 | SEQ ID NO: 82 |
| pGAL4 | SEQ ID NO: 83 |

In some embodiments, the galactose regulation system used to control expression of one or more enzymes of the cannabinoid biosynthetic pathway is re-configured such that it is no longer induced by the presence of galactose. Instead, the gene of interest will be expressed unless repressors, which may be maltose in some strains, are present in the medium.

In some embodiments, the genetic regulatory element is a maltose-responsive promoter. In some embodiments, maltose negatively regulates expression of the cannabinoid biosynthetic pathway, thereby decreasing production of the cannabinoid. In some embodiments, the maltose-responsive promoter is selected from the group consisting of pMAL1, pMAL2, pMAL11, pMAL12, pMAL31 and pMAL32. The maltose genetic regulatory element can be designed to both activate expression of some genes and repress expression of others, depending on whether maltose is present or absent in the medium. Maltose regulation of gene expression and maltose-responsive promoters are described in U.S. Patent Publication 2016/0177341, which is hereby incorporated by reference. Genetic regulation of maltose metabolism is described in Novak et al., “Maltose Transport and Metabolism in S. cerevisiae,” Food Technol. Biotechnol. 42 (3) 213-218 (2004).

TABLE 2
Exemplary MAL Promoter Sequences

| Promoter | Sequence |
|---|---|
| pMAL1 | SEQ ID NO: 84 |
| pMAL2 | SEQ ID NO: 85 |
| pMAL11 | SEQ ID NO: 86 |
| pMAL12 | SEQ ID NO: 87 |
| pMAL31 | SEQ ID NO: 88 |
| pMAL32 | SEQ ID NO: 89 |

In some embodiments, the heterologous genetic pathway is regulated by a combination of the maltose and galactose regulons.

In some embodiments, the recombinant host cell does not contain, or expresses a very low level of (for example, an undetectable amount), a precursor (e.g., hexanoate) required to make the cannabinoid. In some embodiments, the precursor (e.g., hexanoate) is a substrate of an enzyme in the cannabinoid biosynthetic pathway.

### Yeast Strains

In some embodiments, yeast strains useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, chizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the strain is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorphs (now known as Pichia angusta). In some embodiments, the host microbe is a strain of the genus Candida, such as Candida lipolytica, Candida guilliermondii, Candida krusei, Candida pseudotropicalis, or Candida utilis.

In a particular embodiment, the strain is Saccharomyces cerevisiae. In some embodiments, the host is a strain of Saccharomyces cerevisiae selected from the group consisting of Baker's yeast, CEN.PK, CEN.PK2, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the strain of Saccharomyces cerevisiae is CEN.PK.

In some embodiments, the strain is a microbe that is suitable for industrial fermentation. In particular embodiments, the microbe is conditioned to subsist under high solvent concentration, high temperature, expanded substrate utilization, nutrient limitation, osmotic stress due to sugar and salts, acidity, sulfite and bacterial contamination, or combinations thereof, which are recognized stress conditions of the industrial fermentation environment.

### Mixtures

In another aspect, provided are mixtures of the host cells described herein, a culture medium, and an oil overlay. In some embodiments, the oil is a mineral oil. In some embodiments, the oil is a vegetable oil. In some embodiments, the oil overlay includes one or more of soybean oil, sunflower oil, safflower oil, castor oil, ESTEREX™ A51, or JARCOL™ I-16

In some embodiments, the culture medium contains an exogenous agent that decreases production of a cannabinoid. In some embodiments, the exogenous agent that decreases production of the cannabinoid is maltose. In a particular embodiment, the exogenous agent that decreases production of a cannabinoid is maltose.

In some embodiments, the culture medium contains an exogenous agent described herein. In some embodiments, the culture medium contains an exogenous agent that increases production of the cannabinoid. In some embodiments, the exogenous agent that increases production of the cannabinoid is galactose. In some embodiments, the culture medium contains a precursor or substrate required to make the cannabinoid. In some embodiments, the precursor required to make the cannabinoid is hexanoate. In some embodiments, the precursor required to make the cannabinoid is olivetolic acid.

In some embodiments, the culture medium contains an exogenous agent that increases production of the cannabinoid and a precursor or substrate required to make the cannabinoid. In some embodiments, the exogenous agent that increases production of the cannabinoid is galactose, and the precursor or substrate required to make the cannabinoid is hexanoate.

In some embodiments, the culture medium contains a precursor required to make the cannabinoid. In some embodiments, the precursor is hexanoate.

### Methods of Making the Host Cells

In another aspect, provided are methods of making the modified host cells described herein. In some embodiments, the methods include transforming a host cell with the heterologous nucleic acid constructs described herein which encode the proteins expressed by a heterologous genetic pathway described herein. Methods for transforming host cells are described in “Laboratory Methods in Enzymology: DNA”, Edited by Jon Lorsch, Volume 529, (2013); and U.S. Pat. No. 9,200,270 to Hsieh, Chung-Ming, et al., and references cited therein.

### Methods for Producing a Cannabinoid

In another aspect, methods are provided for producing a cannabinoid are described herein. In some embodiments, the method decreases expression of the cannabinoid. In some embodiments, the method includes culturing a host cell comprising at least one enzyme of the cannabinoid biosynthetic pathway described herein in a medium comprising an exogenous agent, wherein the exogenous agent decreases the expression of the cannabinoid. In some embodiments, the exogenous agent is maltose.

In some embodiments, the method results in less than 0.001 mg/L of cannabinoid or a precursor thereof. In some embodiments, the method is for decreasing expression of a cannabinoid or precursor thereof. In some embodiments, the method includes culturing a host cell comprising an AAE, and/or a TKS, and/or a CBGaS, and/or a GPP synthase described herein in a medium comprising an exogenous agent, wherein the exogenous agent decreases the expression of the cannabinoid. In some embodiments, the exogenous agent is maltose. In some embodiments, the method results in the production of less than 0.001 mg/L of a cannabinoid or a precursor thereof.

In some embodiments, the method increases the expression of a cannabinoid. In some embodiments, the method includes culturing a host cell comprising an AAE, and/or a TKS, and/or a CBGaS, and/or a GPP synthase described herein in a medium comprising the exogenous agent, wherein the exogenous agent increases expression of the cannabinoid. In some embodiments, the exogenous agent is galactose. In some embodiments, the method further includes culturing the host cell with the precursor or substrate required to make the cannabinoid.

In some embodiments, the method increases the expression of a cannabinoid or precursor thereof. In some embodiments, the method includes culturing a host cell comprising a heterologous cannabinoid pathway described herein in a medium comprising an exogenous agent, wherein the exogenous agent increases the expression of the cannabinoid or a precursor thereof. In some embodiments, the exogenous agent is galactose. In some embodiments, the method further includes culturing the host cell with a precursor or substrate required to make the cannabinoid or precursor thereof. In some embodiments, the precursor required to make the cannabinoid or precursor thereof is hexanoate. In some embodiments, the combination of the exogenous agent and the precursor or substrate required to make the cannabinoid or precursor thereof produces a higher yield of cannabinoid than the exogenous agent alone.

In some embodiments, the cannabinoid or a precursor thereof is cannabidiolic acid (CBDA), cannabidiol (CBD), cannabigerolic acid (CBGA), or cannabigerol (CBG).

### Culture and Fermentation Methods

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

The methods of producing cannabinoids provided herein may be performed in a suitable culture medium in a suitable container, including but not limited to a cell culture plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric, et al, in Ullmann's Encyclopedia of Industrial Chemistry, Sixth Edition, Volume 12, pages 398-473, Wiley-VCH Verlag GmbH & Co. KDaA, Weinheim, Germany.

In some embodiments, the culture medium is any culture medium in which a genetically modified microorganism capable of producing a heterologous product can subsist, i.e., maintain growth and viability. In some embodiments, the culture medium is an aqueous medium comprising assimilable carbon, nitrogen and phosphate sources. Such a medium can also include appropriate salts, minerals, metals, and other nutrients. In some embodiments, the carbon source and each of the essential cell nutrients are added incrementally or continuously to the fermentation medium, and each required nutrient is maintained at essentially the minimum level needed for efficient assimilation by growing cells, for example, in accordance with a predetermined cell growth curve based on the metabolic or respiratory function of the cells which convert the carbon source to a biomass.

Suitable conditions and suitable medium for culturing microorganisms are well known in the art. In some embodiments, the suitable medium is supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

In some embodiments, the carbon source is a monosaccharide (simple sugar), a disaccharide, a polysaccharide, a non-fermentable carbon source, or one or more combinations thereof. Non-limiting examples of suitable monosaccharides include glucose, galactose, mannose, fructose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol.

The concentration of a carbon source, such as glucose or sucrose, in the culture medium should promote cell growth, but not be so high as to repress growth of the microorganism used. Typically, cultures are run with a carbon source, such as glucose or sucrose, being added at levels to achieve the desired level of growth and biomass. Production of cannabinoids may also occur in these culture conditions, but at undetectable levels (with detection limits being about <0.1 g/l). In other embodiments, the concentration of a carbon source, such as glucose or sucrose, in the culture medium is greater than about 1 g/L, preferably greater than about 2 g/L, and more preferably greater than about 5 g/L. In addition, the concentration of a carbon source, such as glucose or sucrose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium can contain other compounds such as inorganic salts, vitamins, trace metals, or growth promoters. Such other compounds can also be present in carbon, nitrogen, or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate, and mixtures thereof. Typically, the concentration of phosphate in the culture medium is greater than about 1.0 g/L, preferably greater than about 2.0 g/L, and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L, and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances, it may be desirable to allow the culture medium to become depleted of a magnesium source during culture.

In some embodiments, the culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid, and mixtures thereof. Biologically acceptable bases include, but are not limited to, ammonium hydroxide, sodium hydroxide, potassium hydroxide, and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

The culture medium can also include sodium chloride. Typically, the concentration of sodium chloride in the culture medium is within the range of from about 0.1 g/L to about 5 g/L, preferably within the range of from about 1 g/L to about 4 g/L, and more preferably in the range of from about 2 g/L to about 4 g/L.

In some embodiments, the culture medium can also include trace metals. Such trace metals can be added to the culture medium as a stock solution that, for convenience, can be prepared separately from the rest of the culture medium. Typically, the amount of such a trace metals solution added to the culture medium is greater than about 1 mL/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture medium can include other vitamins, such as pantothenate, biotin, calcium, pantothenate, inositol, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The culture medium may be supplemented with hexanoic acid or hexanoate as a precursor for the cannabinoid biosynthetic pathway. The hexanoic acid may have a concentration of less than 3 mM hexanoic acid (e.g., from 1 nM to 2.9 mM hexanoic acid, from 10 nM to 2.9 mM hexanoic acid, from 100 nM to 2.9 mM hexanoic acid, or from 1 μM to 2.9 mM hexanoic acid).

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or production is supported for a period of time before additions are required. The preferred ranges of these components are maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, anti-foaming agent may be added during the culture.

The temperature of the culture medium can be any temperature suitable for growth of the genetically modified cells and/or production of compounds of interest. For example, prior to inoculation of the culture medium with an inoculum, the culture medium can be brought to and maintained at a temperature in the range of from about 20° C. to about 45° C., preferably to a temperature in the range of from about 25° C. to about 40° C. and more preferably in the range of from about 28° C. to about 32° C.

The pH of the culture medium can be controlled by the addition of acid or base to the culture medium. In such cases when ammonia is used to control pH, it also conveniently serves as a nitrogen source in the culture medium. Preferably, the pH is maintained from about 3.0 to about 8.0, more preferably from about 3.5 to about 7.0, and most preferably from about 4.0 to about 6.5.

In some embodiments, the carbon source concentration, such as the glucose concentration, of the culture medium is monitored during culture. Glucose or sucrose concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. As stated previously, the carbon source concentration should be kept below the level at which cell growth inhibition occurs. Although such concentration may vary from organism to organism, for glucose as a carbon source, cell growth inhibition occurs at glucose concentrations greater than at about 60 g/L and can be determined readily by trial. Accordingly, when glucose is used as a carbon source the glucose is preferably fed to the fermenter and maintained below detection limits. Alternatively, the glucose concentration in the culture medium is maintained in the range of from about 1 g/L to about 100 g/L, more preferably in the range of from about 2 g/L to about 50 g/L, and yet more preferably in the range of from about 5 g/L to about 20 g/L. Although the carbon source concentration can be maintained within desired levels by addition of, for example, a substantially pure glucose solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g. the nitrogen and phosphate sources) can be maintained simultaneously. Likewise, the trace metals concentrations can be maintained in the culture medium by addition of aliquots of the trace metals solution.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Characterization of S. cerevisiae Growth in the Presence of Overlay Oil

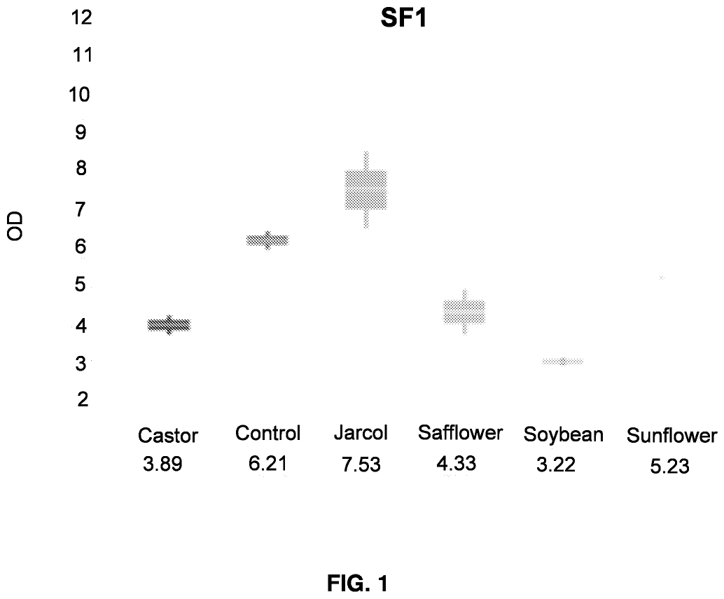

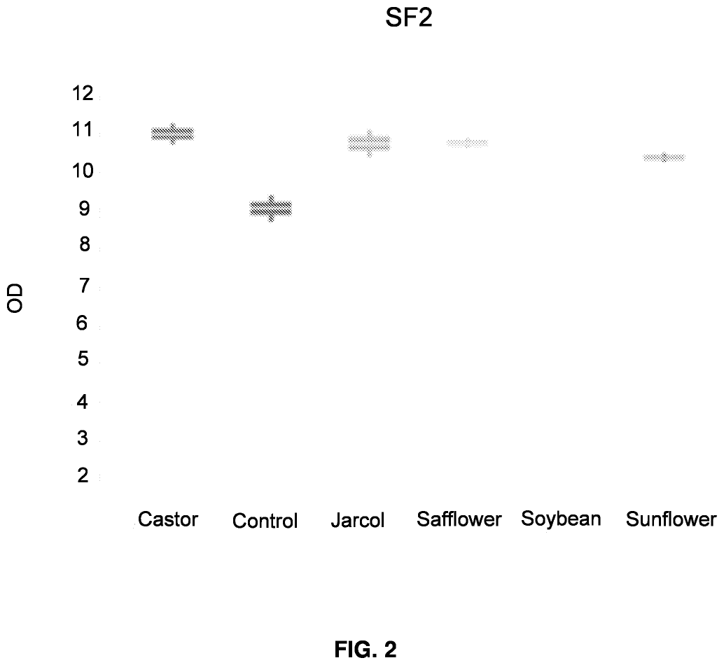

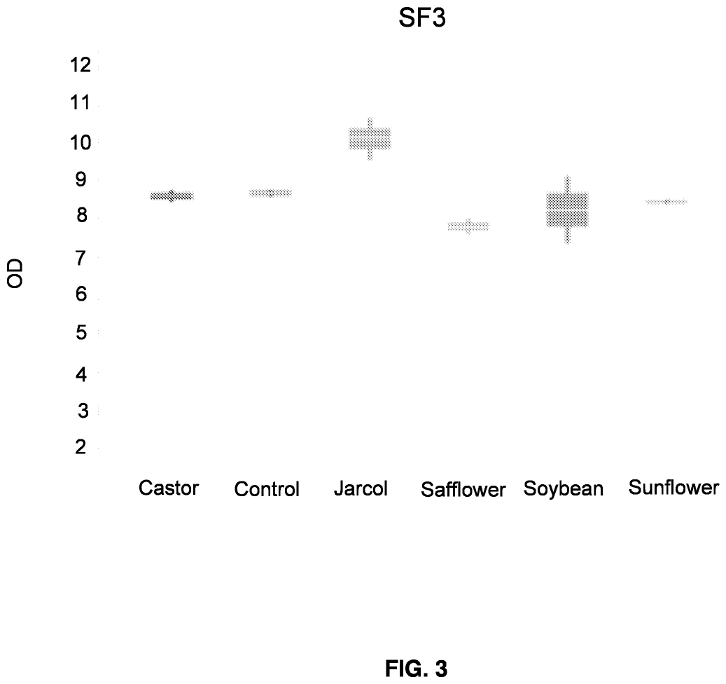

The ability of yeast cells (S. cerevisiae) to grow normally in culture media containing an overlay oil was assessed. Wild-type yeast (strain Y46850) was cultured in the presence of soybean oil, sunflower oil, safflower oil, castor oil, or JARCOL™ I-16. Cell growth was measured by assessing the optical density (OD) of the yeast culture suspensions in three independent experiments (SF1, SF2, and SF3) (FIGS. 1-3). Optical density measurements of wild-type yeast cultured with an overlay oil showed that growth was not significantly different in cultures containing oil compared to control (no oil) samples.

### Example 2. Recovery of a Cannabinoid in the Presence of Overlay Oil

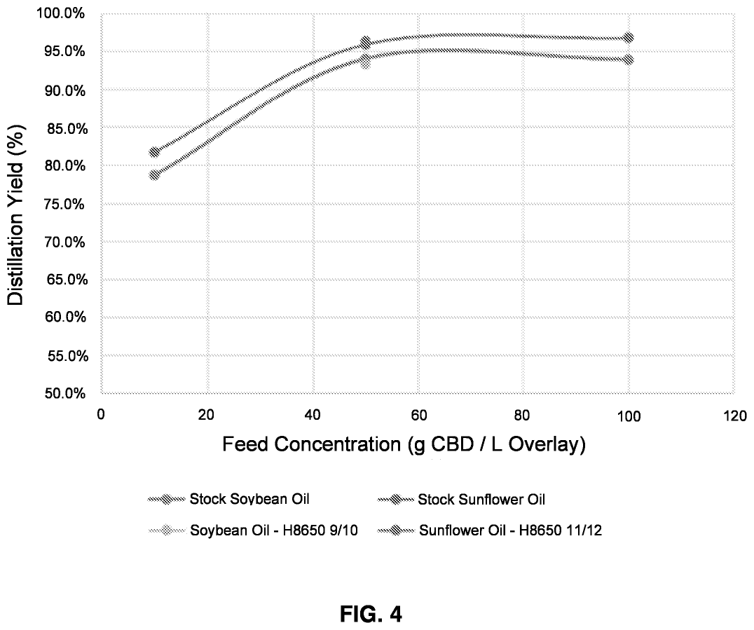

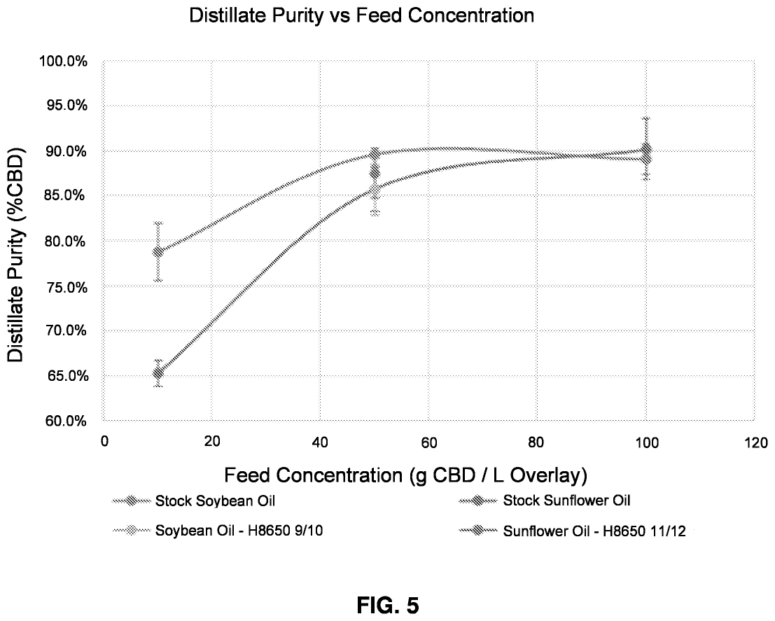

Next, a series of experiments was conducted to evaluate the effect of oil on the extraction of a cannabinoid from a yeast fermentation composition. In a fermentation experiment (H8650), wild-type S. cerevisiae (strain Y46850) were cultured with either 10% sunflower oil or 10% soybean oil (FIGS. 4 and 5). Approximately 10 g/L, 50 g/L, or 100 g/L of cannabidiol (CBD) isolated from an external source was spiked into either the fermentation or into control stock sunflower or soybean oil. The oil was then separated from the fermentation composition and distilled in a wiped film evaporator to characterize the distillation yield (FIG. 4) and the distillate purity (FIG. 5) of the extracted CBD. No significant difference was observed between the distillation yield of CBD extracted from the yeast fermentation compared to CBD extracted from control oil solutions (FIG. 4). Distillation yields of greater than 90% were achieved. Similarly, no significant difference was observed between the distillate purity of CBD extracted from the yeast fermentation compared to the CBD extracted from control oil solutions (FIG. 5). Purity of CBD in excess of 90% was achieved in both scenarios.

### Example 3. Selection of Overlay Oils for Extracting Cannabinoids

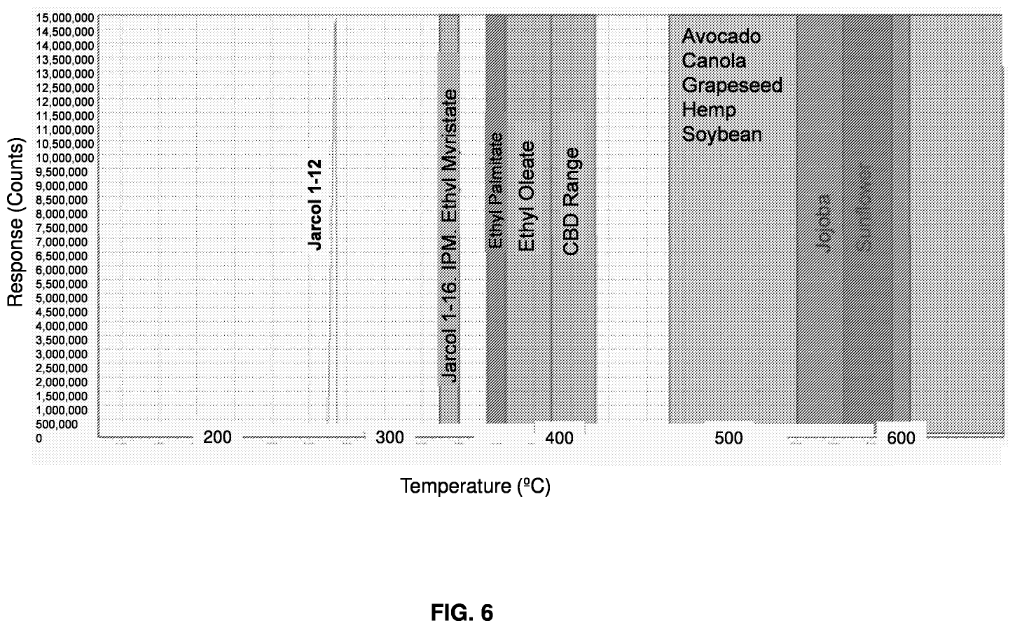

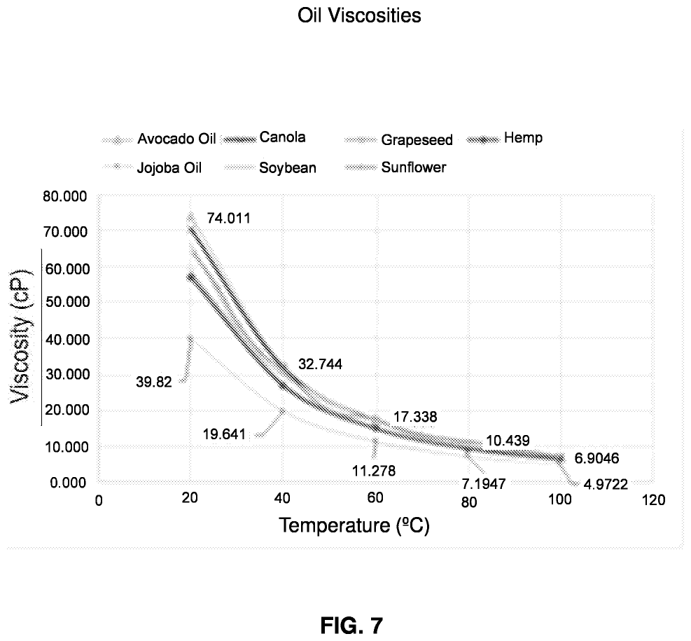

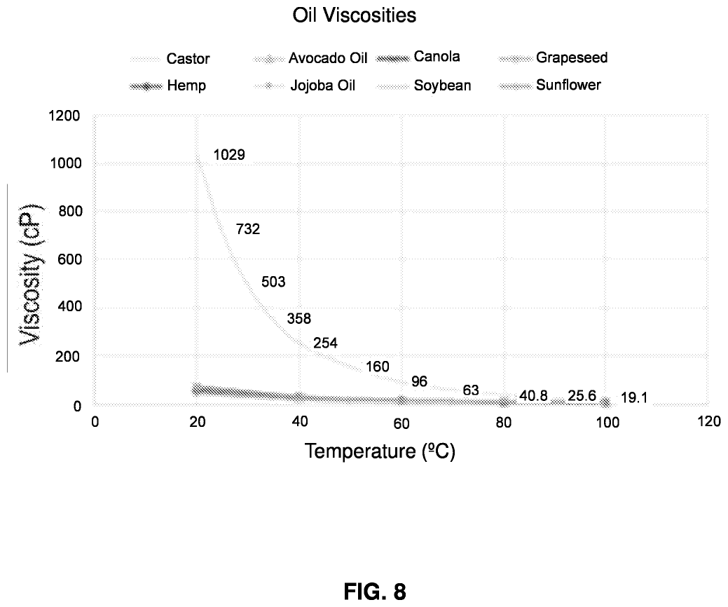

Viscosity, boiling point, food grade status, and cost were the factors that were considered in selecting an overlay oil for the methods of purification of cannabinoids. Simulated distillation boiling points for JARCOL™ |-12, JARCOL™ I-16, IPM, ethyl myristate, ethyl palmitate, ethyl oleate, avocado oil, canola oil, grapeseed oil, hemp oil, soybean oil, jojoba oil, and sunflower oil were compared to the simulated boiling point of CBD. An overlay oil preferably has a boiling point higher than CBD by at least 50° C. in order to achieve extraction. Avocado oil, canola oil, grapeseed oil, hemp oil, soybean oil, jojoba oil, and sunflower oil were identified as having acceptable boiling points (FIG. 6). Next, the viscosities of avocado oil, castor oil, canola oil, grapeseed oil, hemp oil, soybean oil, jojoba oil, and sunflower oil were measured (FIGS. 7 and 8). A target viscosity of less than 300 cP at the temperature range of between 25° C.-30° C. was desired for extraction of cannabinoids. All oils tested met this requirement except for castor oil (FIG. 8). Sunflower oil and soybean oil met all of the requirements in this set of experiments.

### Example 4. Testing Solubility of a Cannabinoid in Overlay Oils

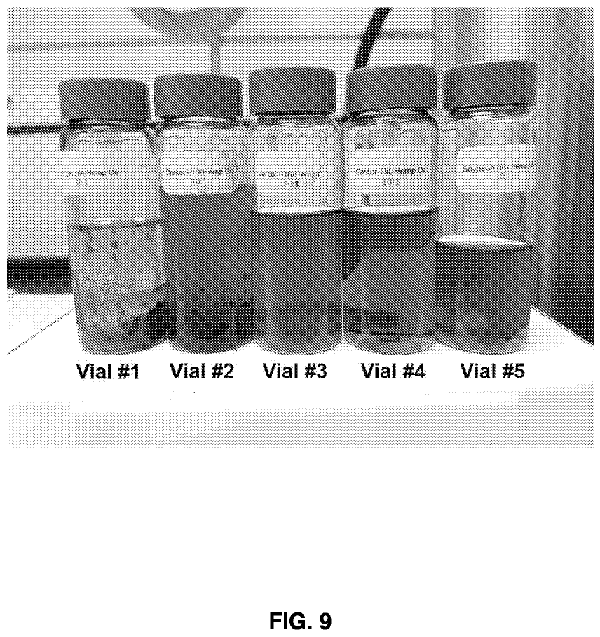

Visual appearances of solutions containing CBDA (as hemp oil with 50% CBDA) were assessed for JARCOL™ I-16, soybean oil, castor oil, ESTEREX™ A 51, sunflower oil, DRAKEOL® 19, and DURASYN® 164. A target CBD concentration of greater than 50 g/L was desired for this set of experiments. Upon visual inspection, it was determined that CBDA was soluble in JARCOL™ I-16, soybean oil, castor oil, ESTEREX™ A 51, and sunflower oil, but was insoluble in DRAKEOL® 19 and DURASYN® 164 at the target concentration. Insolubility was evidenced by the presence of precipitates in solutions of DRAKEOL® 19 or DURASYN® 164 (FIG. 9).

### Example 5. Extraction of a Cannabinoid from a S. cerevisiae Fermentation

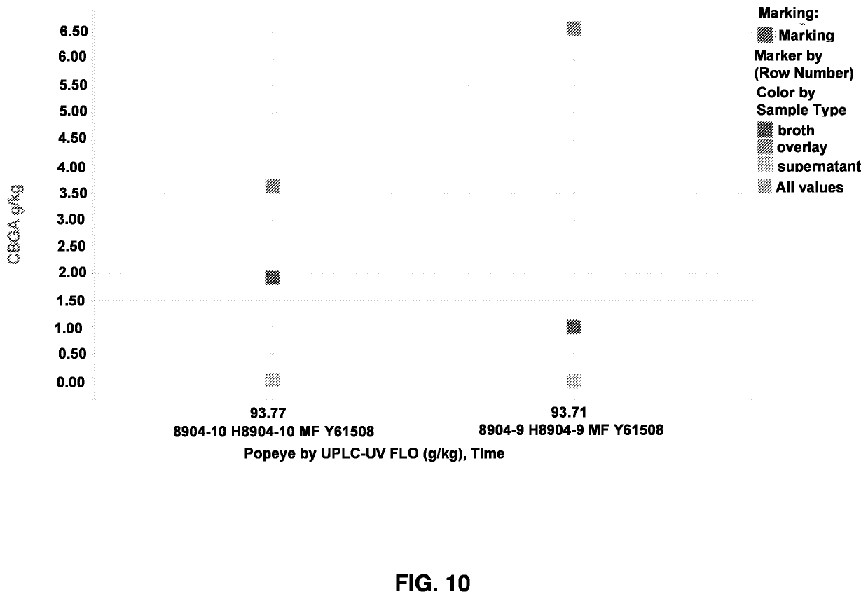

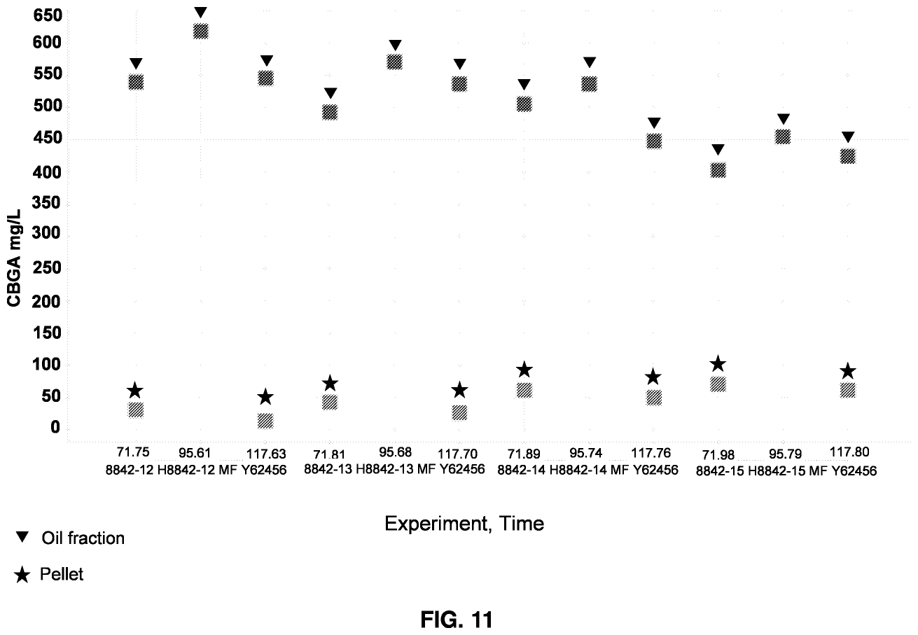

Genetically modified S. cerevisiae that produced CBGA (strain Y61508) were used to produce CBGA in a fermentation composition containing an overlay oil. Fractionation was performed on the fermentation composition to separate the overlay, the broth, and the supernatant fractions. CBGA was found to be present in the overlay oil fraction (FIGS. 10 and 11) in a higher amount compared to the amount present in the aqueous broth, supernatant, or the pellet. The cumulative yield and productivity were measured using CBDA standards due to similarity in signal response between CBDA and CBGA (Tables 3 and 4). The expected concentration of CBGA (Table 4) was calculated using the equation 1. Measured CBGA was obtained following fractionation of the fermentation composition and most of the CBGA was measured in the overlay fraction.

$$[ CGBA ] in overlay = \frac{[ CBGA ] standard}{\% overlay} [ CBGA ] from standard \qquad Eq . 1$$

TABLE 3
Yield and productivity of CBGA in S. cerevisiae (strain
Y61508) fermentation composition containing overlay oil.

| | | | Cum. | Cum. | |
|---|---|---|---|---|---|
| | | Absolute | Yield | Productivity | CBGA |
| Run | Strain | Interval | (%) | (g/L/h) | (g/kg) |
| 9 | Y61508 | Day 0-5 | 0.5868 | 0.011 | 1.10 |
| 10 | Y61508 | Day 0-5 | 1.0942 | 0.02145 | 2.16 |
| 11 | Y61508 | Day 0-5 | 0.615 | 0.01305 | 1.30 |
| 12 | Y61508 | Day 0-5 | 0.6165 | 0.01291 | 1.29 |
| 13 | Y61508 | Day 0-5 | 0.4293 | 0.00945 | 0.94 |
| 14 | Y61508 | Day 0-5 | 0.7015 | 0.01335 | 1.36 |

TABLE 4
Measured and expected CBGA in overlay oil fraction of S. cerevisiae
(strain Y61508) fermentation compositions containing overlay oil.

| | % | Expected CBGA in | Measured CBGA in |
|---|---|---|---|
| Run | Overlay | the Overlay (g/L) | the overlay |
| 9 | 10 | 11.0 | 11.0 |
| 10 | 15 | 14.4 | 26.6 |
| 11 | 10 | 13.0 | 16.6 |
| 12 | 5 | 25.8 | 29.9 |
| 13 | 5 | 18.8 | 23.8 |
| 14 | 10 | 13.6 | 16.4 |

### Example 6. Extraction of a Cannabinoid from a S. cerevisiae Fermentation Using Soybean Oil

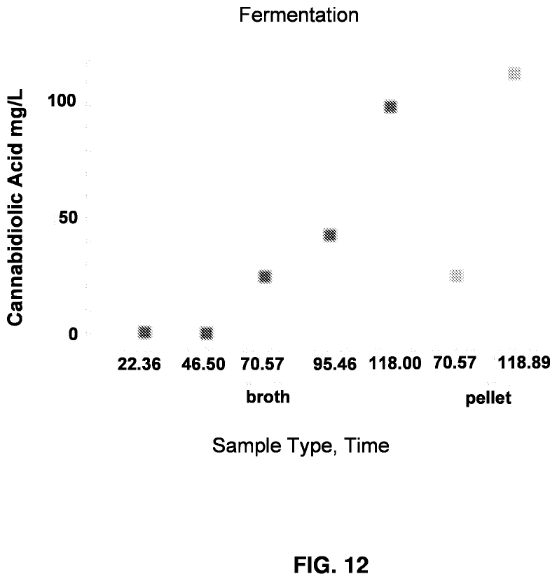

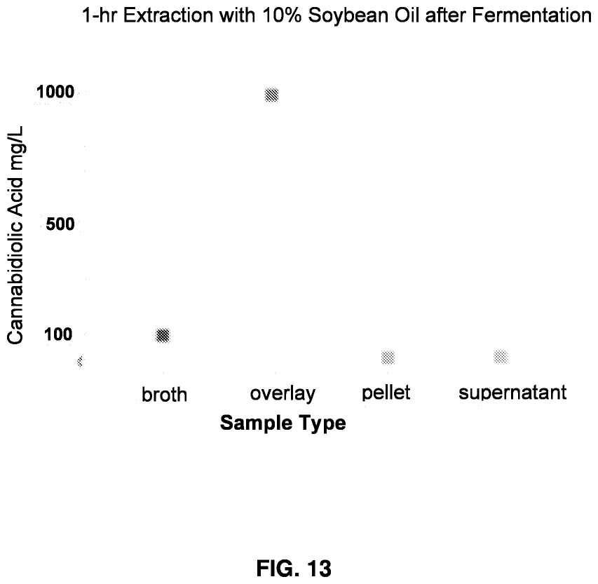

Genetically modified S. cerevisiae that produces CBDA (strain Y64604) were used to produce CBDA in a fermentation composition. In this experiment, fermentation was allowed to run to completion and following fermentation 10% w/w soybean oil was added and mixed in the fermentation for 1 hour. CBDA fermentation was operated without soybean oil overlay for optimal strain performance. In the absence of soybean oil, the CBDA associated with the cell or pellet fraction (FIG. 12). When soybean oil was added and mixed for 1 hour the CBDA was extracted primarily through the oil fraction (FIG. 13).

### Example 7. Effect of Hexanoate on the Yield and Productivity of a Cannabinoid

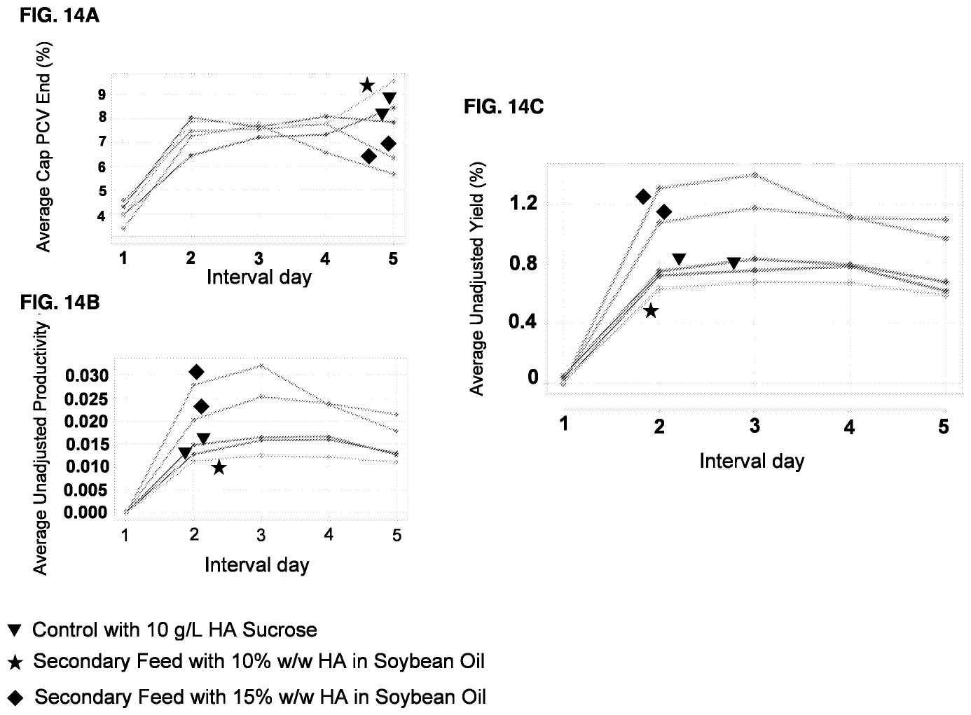

Next, a series of experiments was conducted to evaluate the effects of varying concentrations of hexanoate on the ability of genetically modified yeast to produce CBGA. Blends of 10%, 15%, and 20% hexanoate in soybean oil overlay were contacted with a fermentation composition including S. cerevisiae that were modified to express the enzymes of the CBGA biosynthetic pathway. It was observed that the performance, yield, and productivity were similar, and in some cases higher, with the use of 15% hexanoate compared to sucrose-only control fermentation compositions (FIGS. 14A-14C). Furthermore, hexanoate is miscible in soybean oil, as the solubility of hexanoate in soybean oil was confirmed to be up to ˜185 g/L using gas chromatography (GC-FID). The results of these experiments confirm that including hexanoate (e.g., in concentrations of from about 10% to about 20%) can have a beneficial effect on the production of cannabinoids, such as CBGA, in host cells that are modified to express the enzymes of a cannabinoid biosynthetic pathway and that are contacted with an oil overlay during the fermentation process.

### Example 8. Characterization of CBG Purity and Levels of Impurities

Fermentation of genetically modified S. cerevisiae that produces CBGA (strain Y64618) was performed. In this experiment, the fermentation reaction was allowed to run to completion. The purity of CBGA was measured at 20 minutes, 45 minutes, 72 minutes, 100 minutes, and 116 minutes after the start of the fermentation reaction (Table 5). The abundance of reaction impurities cannabidivarinic acid (CBDVA), cannabidivarin (CBDV), cannabidiolic acid (CBDA), SCBGa, cannabigerol (CBG), tetrahydrocannabivarin (THCV), tetrahydrocannabivarinic acid (THCVA), cannabinol (CBN), cannabinolic acid (CBNA), delta-9-tetrahydrocannabinol (Δ-9-THC), delta-8-tetrahydrocannabinol (Δ-8-THC), cannabicyclol (CBL), cannabichromene (CBC), tetrahydrocannabinolic acid (THCA), cannabichromenic acid (CBCA), and cannabidiol (CBD) was also measured at the timepoints described. At each timepoint, a broth sample was analyzed using HPLC. At 20 minutes, the area under the CBGA peak represented 100% of all compounds of interest in the broth and no detectable levels of impurities were found (Table 5). At approximately 45 minutes from the start of the fermentation, the area under the CBGA peak suggested CBGA accounted for approximately 84.1% of the compounds measured. At approximately 72 minutes from the start of the fermentation, CBGA made up approximately 88.6% of the sample, while SCBGa was approximately 0.9%, and cannabichromene was present at a percentage of approximately 0.28% (Table 5). At approximately 100 minutes from the start of the fermentation, CBGA levels made up approximately 90.4% of the sample, while SCBGa was measured at approximately 1% (Table 5). At approximately 116 minutes from the start of the fermentation, CBGA made up approximately 90.8% of the sample, while SCBGa was approximately 1%, tetrahydrocannabivarinic acid was present at a percentage of approximately 0.1%, and cannabichromene was present at a percentage of approximately 0.6% (Table 5). The concentrations of the various impurities are further characterized in Table 6, below.

TABLE 5
Percent purity of CBGA and impurities
in fermentation of strain Y64618

| Compound | Time (min) | | | | |
|---|---|---|---|---|---|
| Area (%) | 20.17 | 44.96 | 71.54 | 100.18 | 115.94 |
| SCBGa | 0 | 0 | 0.9067 | 0.9567 | 1.0433 |
| CBDVA | 0 | 0 | 0 | 0 | 0 |
| CBDV | 0 | 0 | 0 | 0 | 0 |
| CBDA | 0 | 0 | 0 | 0 | 0 |
| CBGA | 100 | 84.1433 | 88.5967 | 90.38 | 90.8 |
| CBG | 0 | 0 | 0 | 0 | 0 |
| THCV | 0 | 0 | 0 | 0 | 0 |
| THCVA | 0 | 0 | 0 | 0 | 0.1333 |
| CBN | 0 | 0 | 0 | 0 | 0 |
| CBNA | 0 | 0 | 0 | 0 | 0 |
| Δ-9-THC | 0 | 0 | 0 | 0 | 0 |
| Δ-8-THC | 0 | 0 | 0 | 0 | 0 |
| CBL | 0 | 0 | 0 | 0 | 0 |
| CBC | 0 | 0 | 0.28 | 0 | 0.61 |
| THCA | 0 | 0 | 0 | 0 | 0 |
| CBCA | 0 | 0 | 0 | 0 | 0 |
| CBD | 0 | 0 | 0 | 0 | 0 |

TABLE 6
Concentration of CBGA and impurities
in fermentation of strain Y64618

| | Time (min) | | | | |
|---|---|---|---|---|---|
| Compound | 20.17 | 44.96 | 71.54 | 100.18 | 115.94 |
| CBDA | 0 | 0 | 0 | 0 | 0 |
| (mg/l) | | | | | |
| CBGA | 0 | 743.5987 | 2254.1555 | 3058.7221 | 3045.6418 |
| (mg/l) | | | | | |
| THCA | 0 | 0 | 0 | 0 | 0 |
| (mg/l) | | | | | |
| CBDA | 0 | 0 | 0 | 0 | 0 |
| (g/kg) | | | | | |
| THCA | 0 | 0 | 0 | 0 | 0 |
| (g/kg) | | | | | |
| CBGA | 0 | 0.729 | 2.21 | 2.9987 | 2.9859 |
| (g/kg) | | | | | |
| Estimated | 0 | 0 | 0 | 0 | 0.2774 |
| SCBGa | | | | | |
| (mg/l) | | | | | |
| Estimated | 0 | 0 | 0 | 0 | 0.0003 |
| SCBGa | | | | | |
| (g/kg) | | | | | |
| CBD | 0 | 0 | 0 | 0 | 0 |
| (mg/l) | | | | | |
| CBG | 0 | 0 | 0 | 0 | 0 |
| (mg/l) | | | | | |

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

What is claimed is:

1. A method of purifying a cannabinoid, the method comprising:
i) culturing a population of host cells that are genetically modified to express one or more enzymes of a cannabinoid biosynthetic pathway in a culture medium and under conditions suitable for the population of host cells to produce the cannabinoid, thereby producing a fermentation composition,
ii) contacting the fermentation composition with an oil, and
iii) recovering one or more cannabinoids from the fermentation composition or the oil.

2. A method of purifying a cannabinoid, the method comprising:
i) providing a fermentation composition that has been produced by culturing a population of host cells that are genetically modified to express one or more enzymes of a cannabinoid biosynthetic pathway in a culture medium and under conditions suitable for the host cells to produce the cannabinoid;
ii) contacting the fermentation composition with an oil, and
iii) recovering one or more cannabinoids from the fermentation composition or the oil.

3. The method of claim 1 or 2, wherein the host cell comprises one or more heterologous nucleic acids that each, independently, encode (a) an acyl activating enzyme (AAE), and/or (b) a tetraketide synthase (TKS), and/or (c) a cannabigerolic acid synthase (CBGaS), and/or (d) a geranyl pyrophosphate (GPP) synthase.

4. The method of claim 3, wherein the host cell comprises heterologous nucleic acids that independently encode (a) an AAE, (b) a TKS, (c) a CBGaS, and (d) a GPP synthase.

5. The method of claim 3 or 4, wherein the host cell comprises a heterologous nucleic acid that encodes an AAE having an amino acid sequence that is at least 90% identical to the amino acid sequence of any one of SEQ ID NO: 1-24.

6. The method of claim 5, wherein the AAE has an amino acid sequence that is at least 95% identical to the amino acid sequence of any one of SEQ ID NO: 1-24.

7. The method of claim 6, wherein the AAE has the amino acid sequence of any one of SEQ ID NO: 1-24.

8. The method of claim 3 or 4, wherein the host cell comprises a heterologous nucleic acid that encodes an AAE having an amino acid sequence that is at least 90% identical to the amino acid sequence of any one of SEQ ID NO: 1-13.

9. The method of claim 8, wherein the AAE has an amino acid sequence that is at least 95% identical to the amino acid sequence of any one of SEQ ID NO: 1-13.

10. The method of claim 9, wherein the AAE has the amino acid sequence of any one of SEQ ID NO: 1-13.

11. The method of claim 3 or 4, wherein the host cell comprises a heterologous nucleic acid that encodes an AAE having an amino acid sequence that is at least 90% identical to the amino acid sequence of any one of SEQ ID NO: 1-5.

12. The method of claim 11, wherein the AAE has an amino acid sequence that is at least 95% identical to the amino acid sequence of any one of SEQ ID NO: 1-5.

13. The method of claim 12, wherein the AAE has the amino acid sequence of any one of SEQ ID NO: 1-5.

14. The method of any one of claims 1-13, wherein the host cell comprises a heterologous nucleic acid that encodes a TKS having an amino acid sequence that is at least 90% identical to the amino acid sequence of any one of SEQ ID NO: 25-59.

15. The method of claim 14, wherein the TKS has an amino acid sequence that is at least 95% identical to the amino acid sequence of any one of SEQ ID NO: 25-59.

16. The method of claim 15, wherein the TKS has the amino acid sequence of any one of SEQ ID NO: 25-59.

17. The method of any one of claims 1-13, wherein the host cell comprises a heterologous nucleic acid that encodes a TKS having an amino acid sequence that is at least 90% identical to the amino acid sequence of any one of SEQ ID NO: 25-28.

18. The method of claim 17, wherein the TKS has an amino acid sequence that is at least 95% identical to the amino acid sequence of any one of SEQ ID NO: 25-28.

19. The method of claim 18, wherein the TKS has the amino acid sequence of any one of SEQ ID NO: 25-28.

20. The method of any one of claims 1-13, wherein the host cell comprises a heterologous nucleic acid that encodes a TKS having an amino acid sequence that is at least 90% identical to the amino acid sequence of SEQ ID NO: 25.

21. The method of claim 20, wherein the TKS has an amino acid sequence that is at least 95% identical to the amino acid sequence of SEQ ID NO: 25, optionally wherein the TKS has the amino acid sequence of SEQ ID NO: 25.

22. The method of any one of claims 1-21, wherein the host cell comprises a heterologous nucleic acid that encodes a CBGaS having an amino acid sequence that is at least 90% identical to the amino acid sequence of any one of SEQ ID NO: 60-64.

23. The method of claim 22, wherein the CBGaS has an amino acid sequence that is at least 95% identical to the amino acid sequence of any one of SEQ ID NO: 60-64.

24. The method of claim 23, wherein the CBGaS has the amino acid sequence of any one of SEQ ID NO: 60-64.

25. The method of any one claims 1-24, wherein the host cell comprises a heterologous nucleic acid encoding a GPP synthase having an amino acid sequence that is at least 90% identical to the amino acid sequence of any one of SEQ ID NO: 65-70.

26. The method of claim 25, wherein the GPP synthase has an amino acid sequence that is at least 95% identical to the amino acid sequence of any one of SEQ ID NO: 65-70.

27. The method of claim 26, wherein the GPP has the amino acid sequence of any one of SEQ ID NO: 65-70.

28. The method of any one claims 1-24, wherein the host cell comprises a heterologous nucleic acid encoding a GPP synthase having an amino acid sequence that is at least 90% identical to the amino acid sequence of SEQ ID NO: 65.

29. The method of claim 28, wherein the GPP synthase has an amino acid sequence that is at least 95% identical to the amino acid sequence of SEQ ID NO: 65.

30. The method of claim 29, wherein the GPP has the amino acid sequence of SEQ ID NO: 65.

31. The method of any one of claims 3-30, wherein the host cell comprises heterologous nucleic acids that independently encode
a) an AAE having the amino acid sequence of any one of SEQ ID NO: 1-24,
b) a TKS having the amino acid sequence of any one of SEQ ID NO: 25-59,
c) a CBGaS having the amino acid sequences of any one of SEQ ID NO: 60-64, and
d) a GPP synthase having the amino acid sequence of any one of SEQ ID NO: 65-70.

32. The method of any one of claims 1-31, wherein the host cell further comprises one or more heterologous nucleic acids that each, independently, encode an enzyme of the mevalonate biosynthetic pathway, wherein the enzyme is selected from an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

33. The method of claim 32, wherein the host cell comprises heterologous nucleic acids that independently encode an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

34. The method of any one of claims 1-33, the host cell further comprises a heterologous nucleic acid that encodes an olivetolic acid cyclase (OAC).

35. The method of claim 34, wherein the OAC has an amino acid sequence that is at least 90% identical to the amino acid sequence of SEQ ID NO: 71.

36. The method of claim 35, wherein the OAC has an amino acid sequence that is at least 95% identical to the amino acid sequence of SEQ ID NO: 71.

37. The method of claim 36, wherein the OAC has the amino acid sequence of SEQ ID NO: 71.

38. The method of any one of claims 1-37, wherein the host cell further comprises one or more heterologous nucleic acids that each, independently, encode an acetyl-CoA synthase, and/or an aldehyde dehydrogenase, and/or a pyruvate decarboxylase.

39. The method of claim 38, wherein the acetyl-CoA synthase has an amino acid sequence that is at least 90% identical to the amino acid sequence of SEQ ID NO: 72.

40. The method of claim 39, wherein the acetyl-CoA synthase has an amino acid sequence that is at least 95% identical to the amino acid sequence of SEQ ID NO: 72.

41. The method of claim 40, wherein the acetyl-CoA synthase has the amino acid sequence of SEQ ID NO: 72.

42. The method of claim 38, wherein the acetyl-CoA synthase has an amino acid sequence that is at least 90% identical to the amino acid sequence of SEQ ID NO: 73.

43. The method of claim 42, wherein the acetyl-CoA synthase has an amino acid sequence that is at least 95% identical to the amino acid sequence of SEQ ID NO: 73.

44. The method of claim 43, wherein the acetyl-CoA synthase has the amino acid sequence of SEQ ID NO: 73.

45. The method of any one of claims 38-44, wherein the aldehyde dehydrogenase has an amino acid sequence that is at least 90% identical to the amino acid sequence of SEQ ID NO: 74.

46. The method of claim 45, wherein the aldehyde dehydrogenase has an amino acid sequence that is at least 95% identical to the amino acid sequence of SEQ ID NO: 74.

47. The method of claim 46, wherein the aldehyde dehydrogenase synthase has the amino acid sequence of SEQ ID NO: 74.

48. The method of any one of claims 38-47, wherein the pyruvate decarboxylase has an amino acid sequence that is at least 90% identical to the amino acid sequence of SEQ ID NO: 75.

49. The method of claim 48, wherein the pyruvate decarboxylase has an amino acid sequence that is at least 95% identical to the amino acid sequence of SEQ ID NO: 75.

50. The method of claim 49, wherein the pyruvate decarboxylase has the amino acid sequence of SEQ ID NO: 75.

51. The method of any one of claims 3-50, wherein expression of the one or more heterologous nucleic acids is regulated by an exogenous agent.

52. The method of claim 51, wherein the exogenous agent comprises a regulator of gene expression.

53. The method of claim 51 or 52, wherein the exogenous agent decreases production of the cannabinoid.

54. The method of claim 53, wherein the exogenous agent is maltose.

55. The method of claim 51 or 52, wherein the exogenous agent increases production of the cannabinoid.

56. The method of claim 55, wherein the exogenous agent is galactose.

57. The method of claim 56, wherein the exogenous agent is galactose and expression of one or more heterologous nucleic acids encoding the AAE, TKS, and CBGaS enzymes is under the control of a GAL promoter.

58. The method of any one of claims 3-57, wherein expression of one or more heterologous nucleic acids encoding the AAE, TKS, and CBGaS enzymes is under the control of a galactose-responsive promoter, a maltose-responsive promoter, or a combination of both.

59. The method of any one of claims 1-58, further comprising culturing the host cell with a precursor required to make the cannabinoid.

60. The method of claim 59, wherein the precursor required to make the cannabinoid is hexanoate.

61. The method of any one of claims 1-60, wherein the cannabinoid is cannabidiolic acid (CBDA), cannabidiol (CBD) or an acid form thereof, cannabigerolic acid (CBGA), cannabigerol (CBG) or an acid form thereof, tetrahydrocannabinol (THC) or an acid form thereof, or tetrahydrocannabinolic acid (THCa).

62. The method of any one of claims 1-61, wherein the host cell is a yeast cell or yeast strain.

63. The method of claim 62, wherein the yeast cell is S. cerevisiae.

64. The method of any one of claims 1-63, wherein the fermentation composition is separated into a supernatant and a pellet by solid liquid centrifugation.

65. The method of claim 64, wherein the fermentation composition is contacted with the oil after the fermentation is adjusted to a pH of about 8.

66. The method of any one of claims 1-65, wherein the oil is added to the fermentation composition at a concentration of from about 1% to about 25% w/w.

67. The method of claims 66, wherein the oil is added to the fermentation composition at a concentration of about 10% w/w.

68. The method of any one of claims 1-67, wherein the fermentation composition is mixed with the oil for a duration of from about 10 minutes to about 120 minutes.

69. The method of claim 68, wherein the fermentation composition is mixed with the oil for about 60 minutes.

70. The method of claim 68 or 69, wherein the fermentation composition is maintained at a temperature of 55° C.

71. The method of any one of claims 68-70, wherein the fermentation composition is subsequently mixed with the oil for an additional period of time with a duration of from about 1 minute to about 600 minutes.

72. The method of claim 70 or 71, wherein the fermentation composition is maintained at a temperature of 70° C.

73. The method of any one of claims 1-72, wherein the fermentation composition undergoes one or more liquid centrifugation steps after being contacted with the oil.

74. The method of claim 73, wherein the fermentation composition undergoes one or more demulsification steps following each liquid centrifugation step.

75. The method of any one of claims 1-74, wherein the cannabinoid is recovered with a purity of between 50% w/w and 99.9% w/w.

76. The method of claim 75, wherein the recovered cannabinoid has a purity of between 70% w/w and 99.9% w/w.

77. The method of claim 76, wherein the recovered cannabinoid has a purity of between 80% w/w and 99.9% w/w.

78. The method of claim 77, wherein the recovered cannabinoid has a purity of between 90% w/w and 99.9% w/w.

79. The method of claim 75, wherein the recovered cannabinoid has a purity of about 80%, 81%, 82%, 83%, 84%, 85%, 86%, 87%, 88%, 89%, 90%, 91%, 92%, 93%, 94%, 95%, 96%, 97%, 98%, 99%, or 99.9% w/w.

80. The method of any one of claims 1-79, wherein the cannabinoid is recovered with one or more impurities.

81. The method of claim 80, wherein the one or more impurities are present in an amount of from about 0.1% to about 1% w/w.

82. The method of claim 81, wherein the one or more impurities are present in an amount of from about 0.1% to about 0.6% w/w.

83. The method of claim 81, wherein the impurities are present in an amount of about 0.1%, 0.2%, 0.3%, 0.4%, 0.5%, 0.6%, 0.7%, 0.8%, 0.9%, or 1% w/w.

84. The method of any one of claims 80-83, wherein the one or more impurities comprise cannabidivarinic acid, cannabidivarin, cannabidiolic acid, SCBGa, cannabigerol, tetrahydrocannabivarinic acid, cannabinol, cannabinolic acid, delta-9-tetrahydrocannabinol, delta-8-tetrahydrocannabinol, cannabicyclol, cannabichromene, tetrahydrocannabinolic acid, cannabichromenic acid, and/or cannabidiol.

85. The method of any one of claims 1-84, wherein the molar yield of the cannabinoid is between 60% and 100%.

86. The method of claim 85, wherein the molar yield is between 90% and 100%.

87. The method of any one of claims 1-86, wherein the oil comprises a mineral oil, a vegetable oil, a synthetic ester, or a fatty alcohol.

88. The method of claim 87, wherein the oil comprises a vegetable oil.

89. The method of claim 88, wherein the vegetable oil is soybean oil, sunflower oil, safflower oil, canola oil, grapeseed oil, or castor oil.

90. The method of claim 87, wherein the oil comprises a synthetic ester, optionally wherein the synthetic ester is ESTEREX™ A51.

91. The method of claim 87, wherein the oil comprises a fatty alcohol, optionally wherein the fatty alcohol is oleyl alcohol or JARCOL™ I-16.

92. A composition comprising a cannabinoid produced using the method of any one of claims 1-91.

93. The composition of claim 92, wherein the cannabinoid is present with a purity of between 50% w/w and 99.9% w/w.

94. The composition of claim 93, wherein the cannabinoid is present with a purity of about 80%, 81%, 82%, 83%, 84%, 85%, 86%, 87%, 88%, 89%, 90%, 91%, 92%, 93%, 94%, 95%, 96%, 97%, 98%, 99%, or 99.9% w/w.

95. The composition of any one of claims 92-94, wherein the cannabinoid is present in combination with one or more impurities.

96. The composition of claim 95, wherein the one or more impurities are present in an amount of from about 0.1% to about 1% w/w.

97. The composition of claim 96, wherein the one or more impurities are present in an amount of about 0.1%, 0.2%, 0.3%, 0.4%, 0.5%, 0.6%, 0.7%, 0.8%, 0.9%, or 1% w/w.

98. The composition of any one of claims 95-97, wherein the one or more impurities comprise SCBGa, cannabidivarinic acid, cannabidivarin, cannabidiolic acid, cannabigerolic acid, cannabigerol, tetrahydrocannabivarin, tetrahydrocannabivarinic acid, cannabinol, cannabinolic acid, delta-9-tetrahydrocannabinol, delta-8-tetrahydrocannabinol, cannabicyclol, cannabichromene, tetrahydrocannabinolic acid A, cannabichromenic acid, and/or cannabidiol.

99. A composition comprising cannabigerol (CBG), wherein the CBG is produced by a method comprising:
a) culturing a population of host cells that are genetically modified to express one or more enzymes of a CBG biosynthetic pathway in a culture medium and under conditions suitable for the population of host cells to produce CBG thereby producing a fermentation composition; and
b) recovering CBG from the fermentation composition, wherein CBG is present in the composition with a purity of between 50% w/w and 99.9% w/w.

100. The composition of claim 99, wherein the recovered CBG has a purity of between 70% w/w and 99.9% w/w.

101. The composition of claim 100, wherein the recovered CBG has a purity of about 80%, 81%, 82%, 83%, 84%, 85%, 86%, 87%, 88%, 89%, 90%, 91%, 92%, 93%, 94%, 95%, 96%, 97%, 98%, 99%, or 99.9% w/w.

102. The composition of any one of claims 99-101, wherein the CBG is recovered with one or more impurities.

103. The composition of claim 102, wherein the one or more impurities are present in an amount of from about 0.1% to about 1% w/w.

104. The composition of claim 103, wherein the one or more impurities are present in an amount of about 0.1%, 0.2%, 0.3%, 0.4%, 0.5%, 0.6%, 0.7%, 0.8%, 0.9%, or 1% w/w.

105. The composition of any one of claims 99-014, wherein the one or more impurities comprise one or more of SCBGa, cannabidivarinic acid, cannabidivarin, cannabidiolic acid, cannabigerolic acid, cannabigerol, tetrahydrocannabivarin, tetrahydrocannabivarinic acid, cannabinol, cannabinolic acid, delta-9-tetrahydrocannabinol, delta-8-tetrahydrocannabinol, cannabicyclol, cannabichromene, tetrahydrocannabinolic acid A, cannabichromenic acid, and cannabidiol.

106. A fermentation mixture comprising the population of a genetically modified host cell of any one of claims 1-91 and a culture medium.

107. The fermentation mixture of claim 106, wherein the culture medium comprises an exogenous agent and an oil.

108. The fermentation mixture of claim 107, wherein the exogenous agent is hexanoate.

109. The fermentation mixture of any one of claims 106-108, wherein the oil is soybean oil, sunflower oil, safflower oil, canola oil, grapeseed oil, castor oil, ESTEREX™ A51, or JARCOL™ I-16.
